(19) United States
(12) Patent Application Publication
(10) Pub. No.: US 2019/0079090 A1
(43) Pub. Date: Mar. 14, 2019

(54) CELL-SURFACE MOLECULE BINDING STIMULI-RESPONSIVE POLYMER COMPOSITIONS AND METHODS CROSS-REFERENCE TO RELATED APPLICATIONS

(71) Applicant: NEXGENIA, INC., Seattle, WA (US)

(72) Inventors: Barrett J. Nehilla, Seattle, WA (US); Nicole M. Provost, Seattle, WA (US); Matthew J. Manganiello, Seattle, WA (US); Thomas Cox, Redmond, WA (US); Michael Emde, Redmond, WA (US)

(21) Appl. No.: 16/084,546

(22) PCT Filed: Mar. 15, 2017
(86) PCT No.: PCT/US2017/022594

§ 371 Date: Sep. 12, 2018

Related U.S. Application Data
(60) Provisional application No. 62/308,672, filed on Mar. 15, 2016.

(51) Int. Cl. G01N 33/569; G01N 33/536; G01N 33/543; C08L 33/08; C08L 33/26

(52) U.S. Cl. CPC G01N 33/56972; G01N 33/536; G01N 33/54326; C07K 14/7051; C08L 33/08; C08L 33/26; C08L 2203/02; G01N 33/54346

(57) ABSTRACT

Methods and compositions for clustering cells, cell surface molecules, and ligands are disclosed herein. In some versions, the methods include applying a stimulus to a polymer that is reversibly associative in response to the stimulus.

### CROSS-REFERENCE TO RELATED APPLICATIONS

This application claims the benefit under 35 USC 119(e) of prior co-pending U.S. Provisional Patent Application No. 62/308,672, filed Mar. 15, 2016, the disclosure of which is hereby incorporated by reference in its entirety.

### FIELD OF THE INVENTION

The present disclosure relates to stimuli-responsive polymer compositions and methods for using the compositions.

### INTRODUCTION

The adaptive immune system includes the cellular and physiologic processes by which the human body responds to and remembers foreign pathogens. T-lymphocytes (T cells), B-lymphocytes (B cells) and antigen-presenting cells (APCs), such as dendritic cells, are the major cells of the adaptive immune system.

In adaptive immune system function, proteins associated with foreign pathogens are processed into smaller peptide fragments and displayed as peptide antigens on major histocompatibility complex (MHC) molecules on the surface of APCs or infected host cells. The MHC may be referred to as human leukocyte antigen (HLA) when describing the human system. Foreign peptide antigens are recognized by a T cell when its T cell receptor (TCR) binds to the cognate HLA-peptide complex on the APC surface. The MHC-peptide-TCR molecular complexes on the surface of APCs and T cells, in combination with other closely associated cell surface costimulatory and adhesion proteins, are collectively termed the ‘immunological synapse.’ The formation of the immunological synapse entails a restructuring of the cytoskeleton and a relocation and clustering of various cell surface proteins. Formation of the APC-T cell immunological synapse, along with soluble factors sent and received by both cells, activates the T cells allows their clonal expansion and maturation into antigen-specific effector T cells. The antigen-specific effector T cells are of two general classes: CD4+ helper T cells or CD8+ cytotoxic T cells. Both CD4+ and CD8+ T cells are required to elicit robust immune responses to foreign pathogens. For effective T-cell activation, however, a co-stimulatory signal is also provided by the APC and received by the T cell. Therefore, T cell activation and expansion are important processes of the adaptive immune system to protect human hosts from pathogen infections.

### SUMMARY

Methods and compositions for the clustering, co-localization and/or cross-linking, of cell surface molecules, e.g., receptors and their ligands, are disclosed herein. In some versions, the methods include applying a stimulus to a polymer that is reversibly associative in response to the stimulus.

In some versions of the subject embodiments, the methods include contacting a cell with a plurality of binding entities each including an affinity reagent bound to one or more polymers that are reversibly associative in response to a stimulus. In some aspects, the cell includes receptors on a surface and contacting the cell with the binding entities includes binding a plurality of the affinity reagents to cell surface molecules, e.g., receptors. Also, in various instances, the methods include applying a stimulus effective in associating, via self- and co-aggregation, at least some of the plurality of binding entities to one another and thereby clustering cell surface molecules, e.g., receptors, bound to affinity reagents, and/or their ligands. If the cell surface molecules are on different cells, the self- and co-aggregation process as provided herein co-localizes two different cells and/or two different cell types.

Various aspects of the subject methods also include contacting a cell with a binding entity including a plurality of affinity reagents bound to a polymer that is reversibly associative in response to a stimulus, wherein the cell includes molecules, e.g., receptors, on the cell surface and contacting the cell with the binding entity includes binding a plurality of the affinity reagents to cell surface molecules, e.g., receptors. In some aspects, the methods include applying a stimulus effective in associating at least some of the plurality of affinity reagents to one another and thereby clustering, co-localizing and/or cross-linking cell surface molecules, e.g., receptors, bound to affinity reagents, and/or their ligands. If the cell surface molecules are on different cells, the self- and co-aggregation process co-localizes the two different cells and/or two different cell types.

Compositions are also included such as stimuli-responsive reagents. Such reagents may include a binding entity comprising a plurality of affinity reagents bound to a polymer that is reversibly associative in response to a stimulus.

Kits are also included. The kits may include first and, optionally, second compositions each including a binding entity comprising an affinity reagent bound to a polymer that is reversibly associative in response to a stimulus.

### BRIEF DESCRIPTION OF THE DRAWINGS

These and other aspects and features of the present disclosure will become apparent to those ordinarily skilled in the art upon review of the following description of specific embodiments of the disclosures in conjunction with the accompanying figures, wherein:

FIG. 1 provides a list of clusters of differentiation that can be employed according to the subject embodiments.

FIG. 2 provides a list of hormones, cytokines and other growth factors that can be employed according to the subject embodiments.

FIG. 3 illustrates two polymer-affinity reagent conjugates as binding entities, e.g., polymer-Ab conjugates (polymer-Ab conjugate 1 and polymer-Ab conjugate 2), that bind different cell surface receptor classes before stimuli-responsive polymer co-aggregation and receptor clustering.

FIG. 4 provides polymer-affinity reagent conjugates as binding entities, e.g., polymer-Ab conjugates, that bind the same cell surface receptor classes before stimuli-responsive polymer co-aggregation and receptor clustering.

FIG. 5 shows a multivalent binding entity including a plurality of affinity reagents bound to a stimuli-responsive polymer, e.g., a polymer-Ab conjugate, with the same affinity reagents (antibody 1). The conjugate binds to the same cell surface receptor class before stimuli-responsive polymer co-aggregation and receptor cross-linking.

FIG. 6 provides a multivalent binding entity including a plurality of affinity reagents bound to a stimuli-responsive polymer, e.g., polymer-Ab conjugate, with different affinity reagents (e.g., antibody 1 and antibody 2) on a single polymer molecule. The conjugate binds to different cell surface receptor classes before stimuli-responsive polymer co-aggregation and receptor cross-linking.

FIG. 7 provides clustering and multimerization targets for stimuli-responsive polymer-conjugated ligands according to the subject embodiments.

FIG. 8 provides an example of utilizing binding entities comprising polymer-affinity reagent conjugates for clustering a plurality of cells according to the subject embodiments.

FIG. 9 provides an example of utilizing binding entities comprising a plurality of affinity reagents bound to a stimuli-responsive polymer for clustering a plurality of cells according to the subject embodiments.

FIG. 10 illustrates a reaction scheme for conjugating a polymer to an antibody in accordance with the embodiments provided herein.

FIG. 11 provides a gel according to the subject embodiments. The left panel provides unconjugated anti-CD3 and polymer-anti-CD3 conjugate. The right panel provides unconjugated anti-CD28 and polymer-anti-CD28 conjugate.

FIG. 12 provides data obtained in evaluating three different polymer-anti-CD3 conjugates responding to different temperature stimuli according to the subject embodiments.

FIGS. 13A-D are graphs showing structural and functional properties of an embodiment of stimuli-responsive magnetic nanoparticles (mNPs) of the present disclosure. The mNPs include a hydrophilic stimuli-responsive polymer that does not include a micelle-forming group at a proximal terminus of the polymer. FIG. 13A is a graph showing the particle size of six different mNP batches, measured by dynamic light scattering. FIG. 13B is a graph showing a lower critical solution temperature (LCST) of 18° C., a measure of temperature-responsiveness of the mNPs. FIG. 13C is a graph showing the stimuli-responsive polymer to Fe mass ratio of mNPs, as measured by thermogravimetric analysis. FIG. 13D is a graph showing the separation efficiency of the mNP at below (4° C.) and above (24° C.) the LCST.

FIG. 14 provides an example demonstrating that two different polymer-affinity reagent conjugates, e.g., polymer-anti-CD3 and polymer-anti-CD28 conjugates, activate T cells ex vivo. T cells alone do no proliferate, as shown by the single (grey dotted) fluorescence peak. Polymer-conjugated anti-CD3 alone did not induce T cell proliferation, either (black dotted peak). In the presence of polymer-anti-CD3 and polymer-anti-CD28 conjugates, a stimulus caused polymer-Ab co-aggregation and concomitant receptor cross-linking and T cell activation in both CD4+ and CD8+ T cells, as shown by the multiple fluorescence peaks of lower intensities.

FIG. 15 provides an example demonstrating that the same polymer-affinity reagent conjugates, e.g., polymer-anti-CD3 conjugates, activate T cells ex vivo. In the presence of the co-stimulatory signaling molecule IL-2, the T cells proliferated slightly. With polymer-anti-CD3 but not IL-2, the T cells were not activated, as shown by the single sharp fluorescence peak. However, in the presence of polymer-anti-CD3 and IL-2, both CD4+ and CD8+ T cells proliferated and underwent multiple population doublings, as is shown by the multiple fluorescent peaks.

FIG. 16 provides an example demonstrating three different polymer-anti-CD3 conjugates responding to different temperature stimuli activate T cells ex vivo. T cells alone do no proliferate, as shown by the single (dotted) fluorescence peak. In the presence of polymer-anti-CD3 conjugates, a stimulus caused polymer-Ab co-aggregation and concomitant receptor cross-linking and T cell activation, as shown by the multiple fluorescence peaks of lower intensities.

FIG. 17 provides data obtained in evaluating stimuli-responsive mNPs that respond to both temperature and ionic strength.

FIG. 18 provides data obtained in evaluating the isolation of monoclonal antibodies (mAb) from solutions using polymer-protein A conjugates.

FIGS. 19A-C provide data obtained in performing methods described in Example 10 below. FIG. 19A is a graph showing cell numbers as the mean-fold expansion illustrating that T cells expand for at least two weeks after activation via CD3 receptor cross-linking. FIG. 19B is a graph illustrating that after activation via CD3 receptor cross-linking, CD4+ T cells have elevated levels of CD25 co-stimulation marker expression above background (day 0) for over two weeks in culture. FIG. 19C is a graph illustrating that after activation via CD3 receptor cross-linking, CD8+ T cells have elevated levels of CD25 co-stimulation marker expression above background (day 0) for over two weeks in culture.

### DETAILED DESCRIPTION

Methods and compositions for clustering, co-localizing and/or cross-linking cell surface molecules and their ligands are disclosed herein. In some versions, the methods include applying a stimulus to a polymer that is reversibly associative in response to the stimulus.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, various embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, subject methods for applying the subject compositions will be discussed in greater detail, followed by a review of associated compositions.

### I. DEFINITIONS

Terms used in the claims and specification are defined as set forth below unless otherwise specified.

As noted above, the subject disclosure includes methods and compositions for the clustering, co-localization and/or cross-linking, of cell surface molecules, e.g., receptors and their ligands. “Clustering,” “co-localization” and/or “cross-linking,” all refer to an ordered and oriented process in which distant elements (e.g., cell surface molecules) are translocated into closer proximity to one another. At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

As used herein, the term “metal complex” refers to a metal-containing compound that includes a central metal atom or ion and a surrounding array of bound molecules or ions (e.g., ligands).

As used herein, the term “coordinate” or “coordinating” refers to the bonds that form between ligands (e.g., chelating agents) and a central metal atom, where the ligands are generally bound to the central atom by donating electrons from a lone electron pair into an empty metal orbital, such that the ligands are coordinated to the atom. In some embodiments, instead of donating electrons from a lone electron pair into an empty metal orbital, pi-bonds of organic ligands such as alkenes can coordinate to empty metal orbitals.

As used herein, the term “chelating agent” refers to a compound that can form two or more separate coordinate bonds to a central atom.

As used herein, the term “hydrodynamic diameter” refers to the apparent size of soluble stimuli-responsive mNPs hydrated in a solvent (e.g., water), as measured by dynamic light scattering.

As used herein, the term “copolymer” refers to a polymer that is the result of polymerization of two or more different monomers. The number and the nature of each constitutional unit can be separately controlled in a copolymer. The constitutional units can be disposed in a purely random, an alternating random, a regular alternating, a regular block, or a random block configuration unless expressly stated to be otherwise. A purely random configuration can, for example, be: x-x-y-z-x-y-y-z-y-z-z-z . . . or y-z-x-y-z-y-z-x-x . . . . An alternating random configuration can be: x-y-x-z-y-x-y-z-y-x-z . . . , and a regular alternating configuration can be: x-y-z-x-y-z-x-y-z . . . . A regular block configuration has the following general configuration: . . . x-x-x-y-y-y-z-z-z-x-x-x . . . , while a random block configuration has the general configuration: . . . x-x-x-z-z-x-x-y-y-y-y-z-z-z-x-x-z-z-z- . . . .

As used herein, the term “substituted” or “substitution” is meant to refer to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

As used herein, the term “alkyl” refers to a straight or branched chain fully saturated (no double or triple bonds) hydrocarbon (carbon and hydrogen only) group. Examples of alkyl groups include, but are not limited to, methyl, ethyl, propyl, isopropyl, butyl, isobutyl, sec-butyl, tertiary butyl, pentyl and hexyl. As used herein, “alkyl” includes “alkylene” groups, which refer to straight or branched fully saturated hydrocarbon groups having two rather than one open valences for bonding to other groups. Examples of alkylene groups include, but are not limited to methylene, —CH₂—, ethylene, —CH₂CH₂—, propylene, —CH₂CH₂CH₂—, n-butylene, —CH₂CH₂CH₂CH₂—, sec-butylene, and —CH₂CH₂CH(CH₃)—. An alkyl group of this disclosure may optionally be substituted with one or more fluorine groups.

As used herein, the term “aryl” refers to monocyclic or polycyclic (e.g., having 2, 3 or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As used herein, the term “halo” or “halogen” includes fluoro, chloro, bromo, and iodo.

As used herein, the term “fatty acid” refers to a molecule having a carboxylic acid with a long aliphatic chain, which is either saturated or unsaturated.

As used herein, the term “hydrophobic block” refers to a polymer block that includes more hydrophobic constitutional units than hydrophilic constitutional units. Hydrophobic constitutional units are not ionizable in typical aqueous conditions and include one or more hydrophobic moieties (e.g., alkyl group, aryl group, etc.).

As used herein, the term “constitutional unit” of a polymer refers an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “proximal terminus” of a polymer refers to a constitutional unit of the polymer that is positioned at the end of a polymer backbone that coordinates to a metal oxide core of a mNP, once the mNP is formed using the process described herein. The constitutional unit at the end of the polymer backbone (e.g., end group) can be, for example, derived from a monomer unit at the end of the polymer (once the monomer unit has been polymerized), or the end group can be a part of a chain transfer agent or initiating agent that was used to synthesize the polymer.

As used herein, the term “distal terminus” of a polymer refers to a constitutional unit that is positioned at the end of a polymer backbone that is situated away from the proximal terminus of the polymer. In some embodiments, the polymer can have more than one distal termini, such as in the case of a branched polymer, where the distal termini correspond to all the ends of the polymer backbone that are situated away from the proximal terminus of the polymer.

As used herein, the term “micelle-forming group” refers to a group that is capable of forming a micelle in a polar solvent.

As used herein, the term “stimuli-responsive” refers to a material that can respond to changes in external stimuli such as the pH, temperature, UV-visible light, photo-rradiation, exposure to an electric field, ionic strength, and the concentration of certain chemicals by exhibiting property change or any combination of those external stimuli.

As used herein, the term “cell surface molecule” refers to a molecule on a surface of a cell, such as within or on the surface of a cell membrane which is exposed to and contactable by, one or more entity outside the cell. As such, a cell surface molecule may be bindable, e.g., covalently or non-covalently attachable, to one or more entity outside the cell, e.g., a binding entity or ligand.

A “cell surface receptor” is a cell surface molecule which is a receptor, e.g., a molecule that binds a specific soluble ligand, such as an antibody, hormone, cytokine and/or synthetic compound. Cell surface receptors are also referred to herein as receptors.

Also, as used herein, a “subject” can be a “mammal” or “mammalian,” where these terms are used broadly to describe organisms which are within the class mammalia or non-mammalian, which are organisms not within the class. In some embodiments, subjects are humans. While the methods described herein can be applied to perform one or more protocol on a human subject or tissue thereof, it is to be understood that the subject methods can also be carried-out to perform a procedure and/or treatment on other subjects (that is, in “non-human subjects”) or a tissue thereof. Such subjects may include non-mammalian animals, bacteria, viruses, insects or plants. As such, cells according to the embodiments may be cells of tissues of one or more non-mammalian animals, bacteria, viruses, insects or plants.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### II. METHODS EMPLOYING STIMULI-RESPONSIVE POLYMERS & CONJUGATES

Methods of clustering cell surface molecules and/or their ligands are provided herein. The cell surface molecules may be on the same cell, and they may be on one or more different cells or one or more different cell types. The subject methods include activating, expanding or otherwise inducing a cellular response in cells such as T cells, B cells, antigen presenting cells, mast cells, hybridomas, tumor cells, TF-1 and other cell types. Such cells can be collected from a subject or be present within a subject.

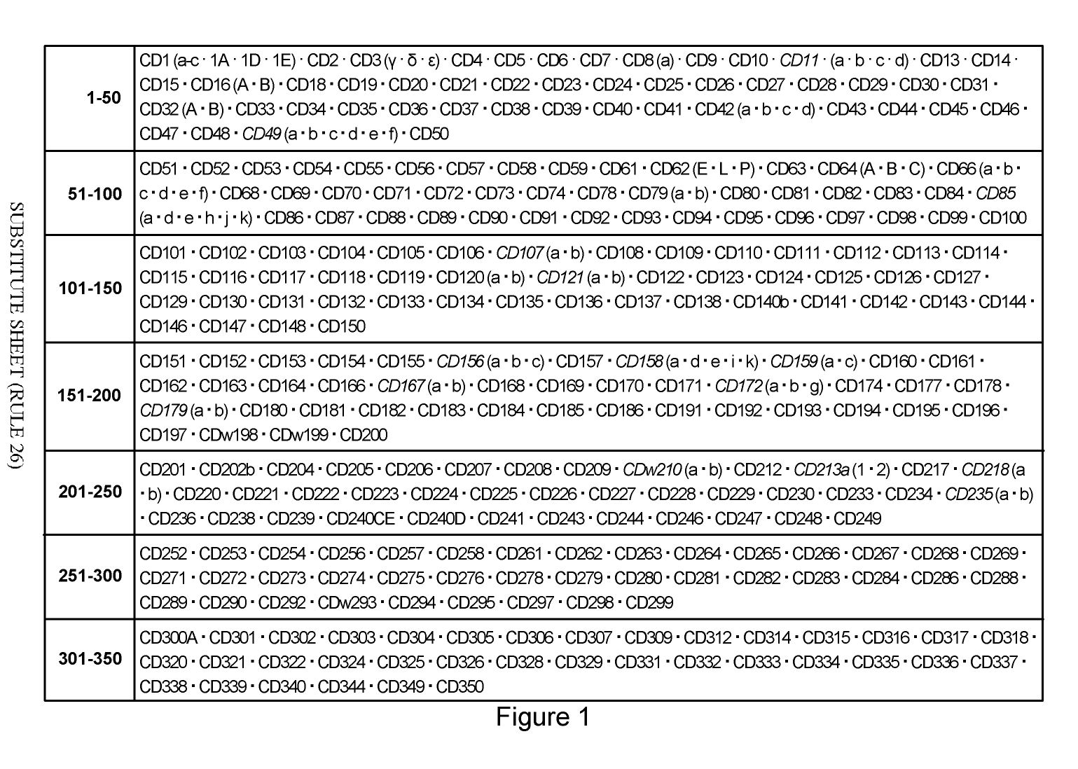

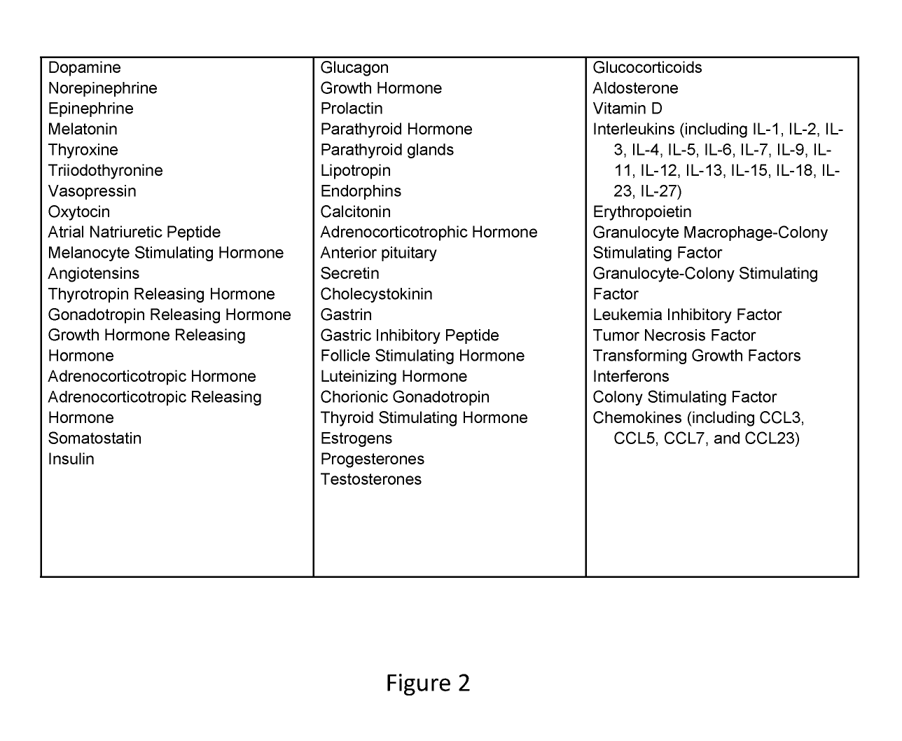

Cell surface molecules according to the subject embodiments include one or more cell surface receptors, such as receptors that bind a specific soluble ligand, such as a hormone or cytokine. Various embodiments of receptors can be T cell receptors, B cell receptors, toll-like receptors and/or cytokine receptors such as granulocyte macrophage colony-stimulating factor (GM-CSF) or Interleukin 3 (IL-3) receptors. Receptors according to the subject embodiments can include first and second receptors on one or more cell and can be, for example, a cluster of differentiation (CD) receptor, such as any one of CD2, CD3, CD4, CD8, CD14, CD19, CD20, CD28, CD30, CD40, CD45, CD116, CD152 and CD169, or any other receptor, e.g., T cell receptor, human leukocyte antigen (HLA), B cell receptor, major histocompatibility complex (MHC), FcεR1, toll-like receptors, provided in the figures or examples of the subject disclosure, or any combination thereof. For example, receptors according to the subject embodiments can include any one or combination of the clusters of differentiation provided in FIG. 1. Cell surface molecules or molecules which bind cell surface molecules as provided herein may also include any of the molecules provided in FIG. 2, or any combination thereof. Cell surface molecules can also include proteins, carbohydrates, or other molecules on a cell surface that are cluster-able or cross-linkable according to the subject methods, or any combination thereof. Cell surface molecules can also include proteins expressed after transduction of cells with viral vectors. Cell surface molecules, in various instances, produce biological effects when clustered or cross-linked because they are in close proximity, even where they do not bind soluble ligands.

Cell surface molecules according to the subject disclosure also can include a receptor that binds or responds to any of the ligands disclosed herein. For example, such molecules can be hormones, cytokines, chemokines, growth factors, small molecules, etc., such as any of the molecules provided in FIG. 2, or any combination thereof.

The presently disclosed subject matter, in various embodiments, includes methods of employing a nanoscale, stimuli-responsive polymer conjugate system comprising the described compositions that can engage and reversibly cluster, co-localize and/or cross-link cell surface molecules, e.g., receptors, according to the subject methods in a controllable manner. The compositions themselves are provided below. Applications of this conjugate system are provided by the subject methods and include the ex vivo activation and expansion of cells, e.g., T and/or B cells, or the induction of other cellular responses, for adoptive cell therapy, other therapeutic areas and/or research use.

In various embodiments, the methods include contacting a cell or a portion thereof, e.g., a cell surface, with a plurality of binding entities each including an affinity reagent, e.g., only one single affinity reagent, bound to one or more polymers that are reversibly associative in response to a stimulus. Such contacting may be performed by placing the cell and the binding entities together in a solution, e.g., a liquid solution including water and/or buffer. Also, in some versions the methods include contacting a cell with a binding entity including a plurality of affinity reagents bound to a polymer that is reversibly associative in response to a stimulus. In various embodiments, the cells are T cells. Such T cells can be, but are not limited to, CD4+ helper T cells and/or CD8+ cytotoxic T cells.

In some versions, a cell includes cell surface molecules, e.g., receptors, on a surface and contacting the cell with the binding entities includes binding a plurality of the affinity reagents to one or more of the cell surface molecules, e.g., receptors. Receptors can include, for example, B cell and/or T cell receptors, such as CD19, CD3 and/or CD28 receptors. Also, in some aspects, the methods include applying a stimulus effective in associating at least some of the plurality of binding entities to one another and thereby clustering, such as by decreasing a length between, cell surface molecules bound to affinity reagents.

As such, a component of the presently disclosed subject matter is an affinity reagent, e.g., an antibody (Ab), that is covalently conjugated to one or more stimuli-responsive polymers to make a polymer-affinity reagent conjugate, e.g., polymer-Ab conjugate. Affinity reagents can, in various embodiments, include a variety of biomolecules such as cytokines, Abs, hormones, oligonucleotides, lipids, and/or enzymes, etc. After conjugation with stimuli-responsive polymers, the affinity reagent adopts similar stimuli-responsive behavior to that of the polymers.

The polymer-conjugated affinity reagents efficiently diffuse in their soluble state and bind to targets (e.g., cell surface molecules) on cell surfaces. After a stimulus is applied, e.g., a temperature shift or pH change within physiological range, according to the subject methods, the surface molecule-bound polymer-conjugated affinity reagents rapidly co-aggregate. Co-aggregation of the polymer-conjugated affinity reagents brings the cell surface molecules, e.g., receptors, in close proximity on the cell surface, allowing their physiologic clustering, co-localization and/or cross-linking. Clustering, co-localization and cross-linking will be used interchangeably. As an example, CD3 receptor clustering in the presence of a co-stimulatory signal leads to T cell activation and subsequent T cell expansion. The inducible aggregation of these stimuli-responsive polymer affinity reagent conjugates provides an ideal scaffold for inducing T cell activation and expansion without the need of exogenously added particulate beads. However, in some embodiments, magnetic nanoparticles are exogenously added. In such instances, the function of the polymer affinity reagent conjugates may be magnetic nanoparticle-independent. The cross-linking of T cell receptors (TCRs) and/or costimulatory receptors is a necessary signal for T cell activation. Importantly, the stimulus-induced aggregation is rapidly and wholly reversible according to the subject methods, so when the stimulus is reversed, such as by a reversion to the original pH and/or temperature, the polymer-Ab conjugates disaggregate.

According to disclosed methods, receptors on T cell surfaces are clustered by polymer-Ab conjugates for activation. Such methods can also be used to control the solution behavior of other polymer-ligand conjugates, increasing their local concentration near cell surface receptors, and increasing the efficiency and potency of binding of signaling molecules such as cytokines and hormones to their cognate receptors. This can include an increase in binding affinity and avidity of the ligands to their cognate receptors. As such, the methods include multimerization of cytokine, hormone and other receptors, and their ligands, on cell surfaces.

Promoting multimerization of receptors for enhanced cell signaling is particularly important for low affinity receptor-ligand interactions. If a ligand has low affinity for a cell surface receptor, the ligand may engage the receptor but disengage soon afterward, resulting in little or no signal transduction. The localized environment is changed when polymer-ligand conjugates are aggregated around clustered cell surface receptors according to the subject methods. For example, polymer-ligand conjugates can be exposed to cells, allowing their binding to individual cell surface receptors. A stimulus will co-aggregate the polymer-ligand conjugates and cluster the cell surface receptors. Then, if a single ligand-receptor interaction is disrupted, there are many other (aggregated) polymer-ligand conjugates in close proximity to the receptor for rapid re-engagement. Therefore, the avidity of the interaction is increased and cell signaling is enhanced by employing the disclosed methods, even though the affinities of the individual ligand-receptor interactions remain low.

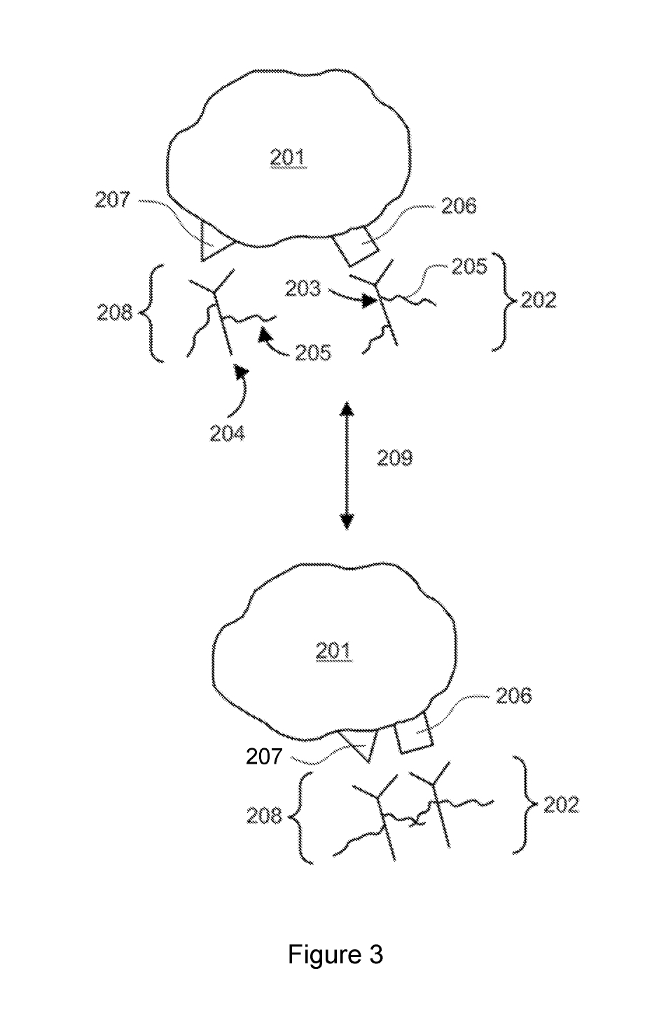

In some versions of the subject methods, and as is illustrated in FIG. 3, contacting a cell 201, e.g., a T-cell, with a plurality of binding entities 202, 208, e.g., polymer-Ab conjugates, each including an affinity reagent 203, 204, such as an antibody, bound to a polymer 205 that is reversibly associative in response to a stimulus. Such binding entities include a first affinity reagent 203, such as anti-CD3, and a second affinity reagent 204, such as anti-CD28, different than the first affinity reagent 203 and/or the cell surface molecules, e.g., receptors, include a first cell surface molecule, e.g., receptor, 206, such as CD3, and a second cell surface molecule, e.g., receptor, 207, such as CD28, different than the first cell surface molecule, e.g., receptor. Furthermore, in some aspects, binding the affinity reagents to the cell surface molecules, e.g., receptors, includes binding the first affinity reagent to the first cell surface molecule, e.g., receptor, and binding the second affinity reagent to the second cell surface molecule, e.g., receptor. As such, in some versions a cell includes different types of cell surface molecule, e.g., receptor, on its surface. Binding entities 202 and 208, bound to their cognate surface molecules 206 and 207, can then associate (co-aggregate) with one another and/or other bound binding entities when exposed to a reversible stimulus, as illustrated by arrow 209. Such association, or co-aggregation, will in turn bring the different types of cell surface molecules, e.g., receptors, in closer proximity to one another on the cell surface.

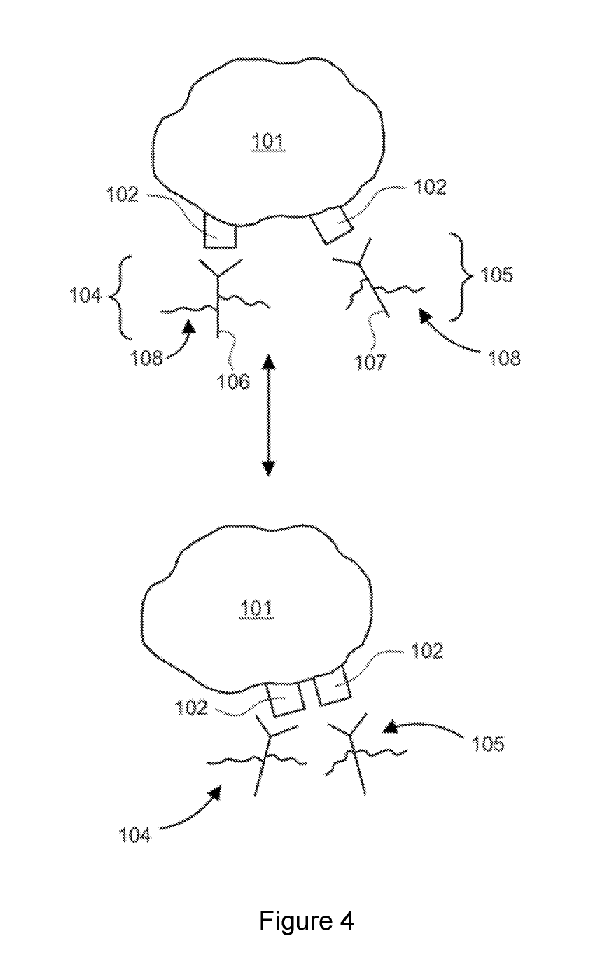

As is illustrated, for example, in FIG. 4, which includes many of the same elements as FIG. 3, such a method can also be applied for cell surface molecules, e.g., receptors 102, on a surface of a cell 101 that are of the same type, such as a cell surface molecule, e.g., CD3. In such an embodiment, the binding entities 104, 105 may include the same or different types of affinity reagents 106, 107, e.g., anti CD-3 antibodies binding to the same or different regions of receptor 102, e.g. CD3. Binding entities 104, 105, e.g., polymer-Ab conjugates, each include an affinity reagent 106, 107, such as an antibody, bound to one or more polymers 108 that are reversibly associative in response to a stimulus. Furthermore, in some aspects, binding a plurality of the affinity reagents 106, 107 to the cell surface molecules, e.g., receptors 102, includes binding the first affinity reagent 106 to cell surface molecule, e.g., receptor 102, and binding the second affinity reagent to surface molecule, e.g., receptor 102. Binding entities 104, 105, can bind to each surface molecule 102, which can then associate (co-aggregate) with other bound binding entities when exposed to a reversible stimulus, as illustrated by arrow 109. Such association, or co-aggregation, will in turn bring the cell surface molecules, e.g., receptors 102, in closer proximity to one another on the cell surface.

These described binding entities can be employed for different applications according to the subject methods. Different affinity reagents can be conjugated to stimuli-responsive polymers as separate reagents, e.g., polymer-Ab conjugate #1 and polymer-Ab conjugate #2, and they can then be combined for use in assays or therapies. For example, polymer-Ab conjugate #1 can include an agonistic Ab specific for the T cell receptor and polymer-Ab conjugate #2 can include an Ab that transmits a co-stimulatory signal to the T cell. After binding to cell surface receptors, co-aggregation of the two polymer-Ab conjugates results in TCR and co-stimulatory receptor cross-linking, providing a powerful activation signal to the T cell.

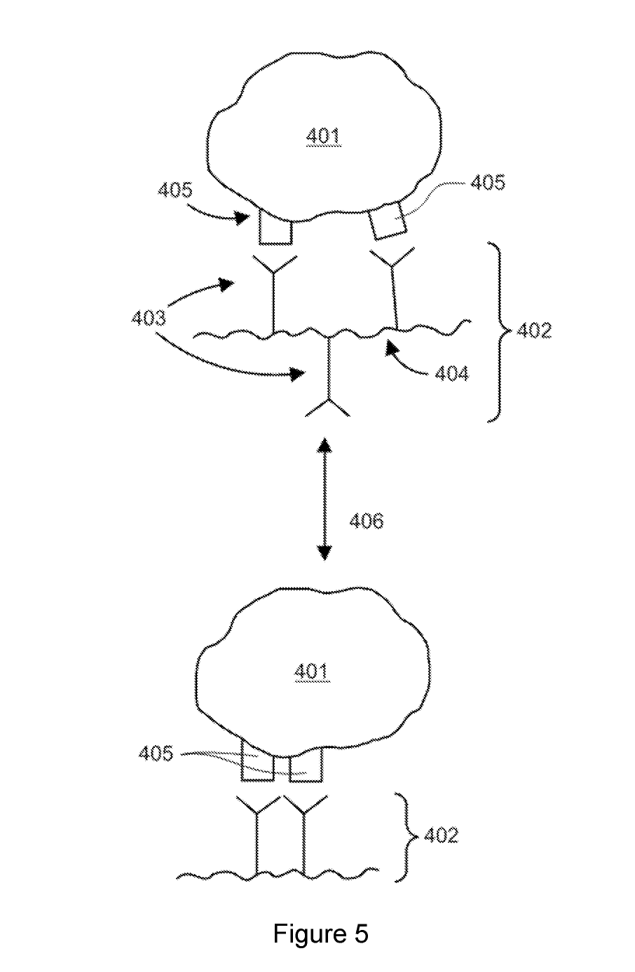

Also as is shown, for example, in FIG. 5, the methods also include contacting a cell 401, e.g., a T-cell, with a binding entity 402, such as a multivalent polymer-antibody conjugate, including a plurality of affinity reagents 403, e.g., antibodies, such as anti-CD3, bound to a polymer 404 that is reversibly associative in response to a stimulus. The cell 401 specifically includes cell surface molecules, e.g., receptors, 405, such as CD3 receptor, on its surface. Contacting the cell 401 with the binding entities 402 includes binding a plurality of the affinity reagents 403 to cell surface molecules, e.g., receptors, 405. The methods also include applying a removable stimulus, as illustrated by arrow 406 effective in associating at least some of the plurality of affinity reagents 403 to one another, to, for example, co-localize the cell surface molecules, e.g., receptors, and thereby cluster cell surface molecules, e.g., receptors, 405 bound to affinity reagents 403 produced by an aggregated multivalent polymer-Ab conjugate. In some versions of the embodiments, such as that provided in FIG. 5, the cell surface molecules, e.g., receptors, are of the same type, e.g., CD3 receptors or CD28 receptors, and/or the affinity reagents are of the same type, e.g., anti CD-3 or anti CD-28.

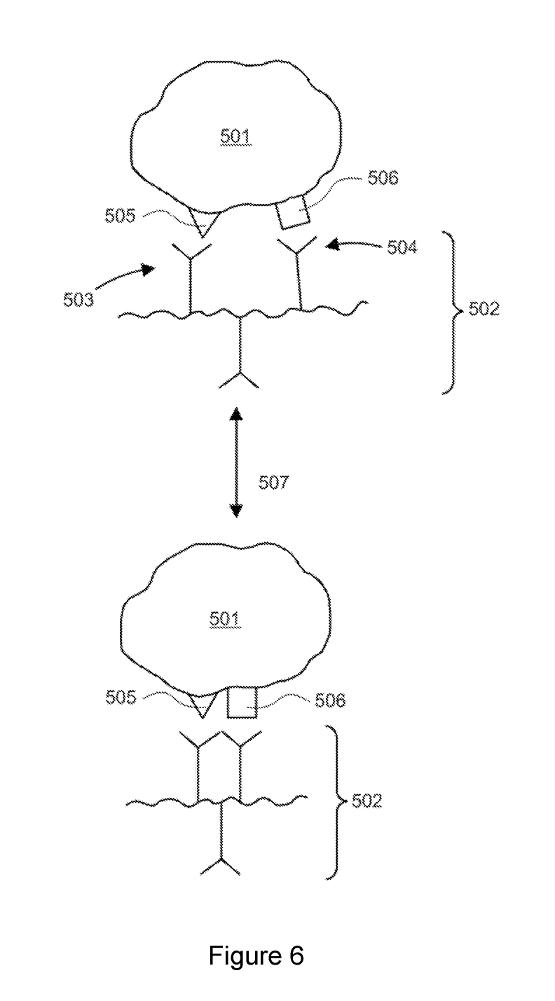

Also, in some versions, such as that shown in FIG. 6, the binding entity 502 includes a first affinity reagent 503, e.g., anti-CD3, and a second affinity reagent 504, e.g., anti-CD28, different than the first affinity reagent. Also, in some versions the cell surface molecules, e.g., receptors, of a cell 501 include a first cell surface molecule, e.g., receptor, 505, e.g., CD3, and a second cell surface molecule, e.g., receptor, 506, e.g., CD28, different than the first cell surface molecule, e.g., receptor, and wherein binding the affinity reagents to the receptors includes binding the first affinity reagent to the first cell surface molecule, e.g., receptor, and binding the second affinity reagent to the second cell surface molecule, e.g., receptor. The cell surface molecules, e.g., receptors, can be clustered in response to a reversible stimulus, as shown by the arrow 507.

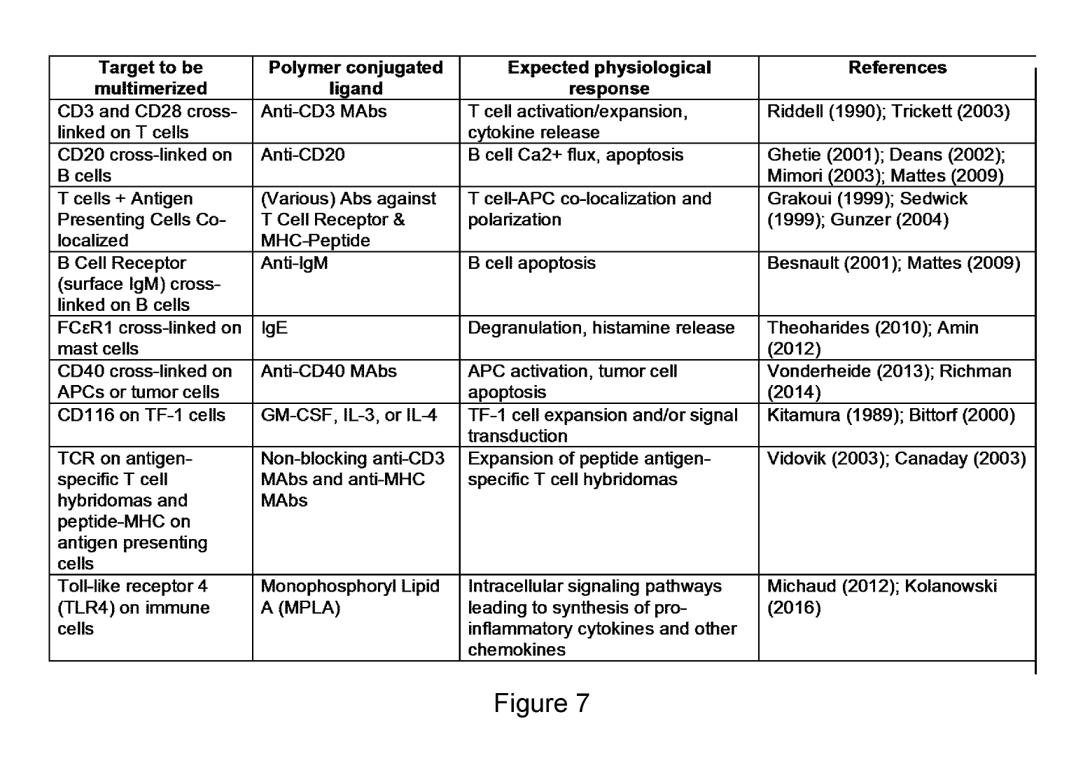

Various clustering and multimerization targets for stimuli-responsive polymer-conjugated ligands according to the subject embodiments are provided by FIG. 7. Specifically provided are targets to be clustered, cross-linked or multimerized, polymer conjugated ligands that could serve as binding entities and expected physiological responses by cells when the binding entities are co-aggregated in response to a reversible stimulus.

In another aspect, the disclosed subject matter includes methods for immobilizing particles, e.g., magnetic or non-magnetic nanoparticles on, and releasing particles from, a substrate or target cell surface. In one embodiment, the method includes the steps of: (a) contacting a cell with a plurality of particles, wherein each particle includes a polymer that is reversibly associative in response to a stimulus; and/or (b) contacting the cell and/or substrate with a plurality of polymer-conjugated monovalent or multivalent binding entities; and/or (c) applying a stimulus effective in associating at least some of the plurality of particles to binding entities that are bound to cell surface molecules to immobilize at least some of the particles to provide immobilized particles, wherein the immobilized particles are immobilized on the target cell through an associative interaction with polymer. In one embodiment, a magnetic field is then applied to isolate the target cells. In one embodiment, each particle further includes a target binding partner. In one embodiment, the method further includes removing the stimulus effective in immobilizing the particle to the cell, thereby reversing the associative interaction between the polymer on the binding entity and the polymer on the particle and releasing the particles from the cell.

In the method, a substrate may be modified to include a polymer that is reversibly associative in response to a stimulus. Alternatively, the substrate can inherently have the characteristic (e.g., hydrophobicity) of association with the polymer in its associative state.

It is also possible to use different stimuli-responsive polymers in the system. That is, the biomolecule may be conjugated to one polymer and the particle surface may be coated with another, permitting control of their reversible adherence by two different signals. Similarly, the walls or surfaces of the substrates may be coated with a polymer that is different from that which is conjugated to the biomolecule or particle surface, again providing for reversible separation control by two different signals. One can also envisage gradients of polymer composition on surfaces, permitting gradually increasing or decreasing strength of adherence along the length or over the area of the device, depending on the conditions.

The selective separation of biomolecules, particles or cells can not only be carried out in the channels of a microfluidic device such as a lab on a chip, but it could also be used on the surface of a surface plasmon resonance (SPR) analytical device, biochips, microarrays, chromatography columns, chromatography resins, filters, and other devices, as well as imaging and therapeutic particle systems and tubes.

Polymers that undergo phase transitions in response to environmental stimuli such as temperature and pH may be applied for drug delivery, separations, and therapeutic or diagnostic applications. One temperature-responsive class is based on alkyl acrylamide polymers, such as poly(N-isopropylacrylamide) (poly(NIPAM)), which undergoes a sharp coil-globule transition and phase separation at its lower critical solution temperature (LCST) in water. This spontaneous process is endothermic and is therefore driven by a gain in entropy associated with the release of hydrophobically-bound water molecules. The LCST of such thermally-sensitive polymers can be tuned to a desired temperature range by copolymerization with a more hydrophilic comonomer (which raises the LCST) or a more hydrophobic comonomer (which lowers the LCST).

As noted above, in various embodiments, the subject disclosure includes performing T cell activation, expansion and/or proliferation. T cell activation and expansion are processes of the adaptive immune system which protect human hosts from pathogen infections. Such activation and/or expansion may result from clustering and co-localizing cell surface molecules, e.g., receptors. In addition, for optimal T-cell activation, a co-stimulatory signal can also be provided by co-localizing both the APC and the T cell surface molecules, and the subject methods include providing such a signal.

In addition, the subject methods include, in some versions, performing T-cell activation, expansion and/or proliferation ex vivo. T-cell activation, expansion and proliferation may be applied to control the ex vivo manufacture of adoptive cell therapies for cancer and viral infections. In the manufacturing process of adoptive cell therapies, sufficient numbers of cells may be grown ex vivo before infusion of the therapeutic cells into the patient. In the field of adoptive cell therapies, a focus has been placed on chimeric antigen receptor (CAR) T cells.

Manufacturing T cells genetically engineered to express CARs or T cell receptors (TCRs) is a multi-step process, and may require reagents to isolate and activate T cells. A generalized approach to CAR T cell therapy involves the following steps: Peripheral blood mononuclear cells (PBMCs) are collected from patients by apheresis; T cells are isolated, activated with anti-CD3/CD28 antibody (Ab)-coated magnetic beads or other T cell activation approaches, and then transduced with a vector encoding the CAR that targets the diseased cells in the patient. These genetically engineered T cells are then expanded over many days to weeks in order to yield sufficient cells for a therapeutic dose, and the expanded CAR T cells are infused into the patient.

According to the subject embodiments, stimuli-responsive polymer-Ab conjugates are applied for cell surface molecule, e.g., receptor, clustering. The Abs can be conjugated separately, as distinct reagents, and later combined for cell-based assays or therapies. Also, according to the subject disclosure, it is the co-aggregation of polymer-Ab conjugates bound to T cell surface molecules that clusters T cell receptors and stimulates T cell receptor cross-linking, and subsequent cell activation. The co-aggregation of polymer-Ab or polymer-ligand conjugates caused by the application of a stimulus such as a thermal or chemical stimulus is completely reversible, such as by dis-aggregation. Furthermore, the polymer-Ab conjugates, as set forth herein, may be monovalent and as such, include a single Ab or ligand with one or multiple polymers conjugated to it, or multivalent and as such, include one or multiple types of Abs or ligands.

Furthermore, in some versions of the embodiments, the methods include clustering, such as by applying a stimulus as described herein, and thereby increasing an effective local concentration of a molecule, e.g., GM-CSF, available to bind a receptor such as a CD116 GM-CSF receptor. For example, clustering surface molecules, such as receptors, can increase a concentration of corresponding binding molecules, e.g., GM-CSF, in a solution proximate, such as within a distance wherein a binding interaction can freely occur, the cluster. In such an aspect, if a binding interaction between a surface molecule is disrupted, there are a plurality of other molecules in close proximity to the surface molecule which can rapidly re-engage the surface molecule. Accordingly, such a method includes increasing the avidity of the interaction and/or enhancing cell signaling. Such avidity can be increased and/or signaling enhanced even if the affinity of an individual ligand-receptor interaction remains relatively low.

Furthermore, in yet other versions of the embodiments, the methods include clustering, such as by applying a stimulus as described herein, and thereby increasing an effective local concentration of a molecule, e.g., MPLA, available to bind a receptor such as a TLR4 receptor. For example, as described above, clustering surface molecules, such as receptors, can increase a concentration of corresponding binding molecules, e.g., MPLA, in a solution proximate, such as within a distance wherein a binding interaction can freely occur, the cluster. In addition, the stimuli-responsive behavior of the polymer-conjugated ligands can be applied as an “on-off” switch for cell signaling.

Also provided herein are methods for clustering two or more cells which may be the same or different cell types. Each of such cells may have any of the cell types provided herein, or any combination thereof. For example, each of the cells for clustering may be T cells. In other embodiments, one cell for clustering is a T cell and one or more other cells for clustering are not T cells, such as antigen presenting cells or B cells.

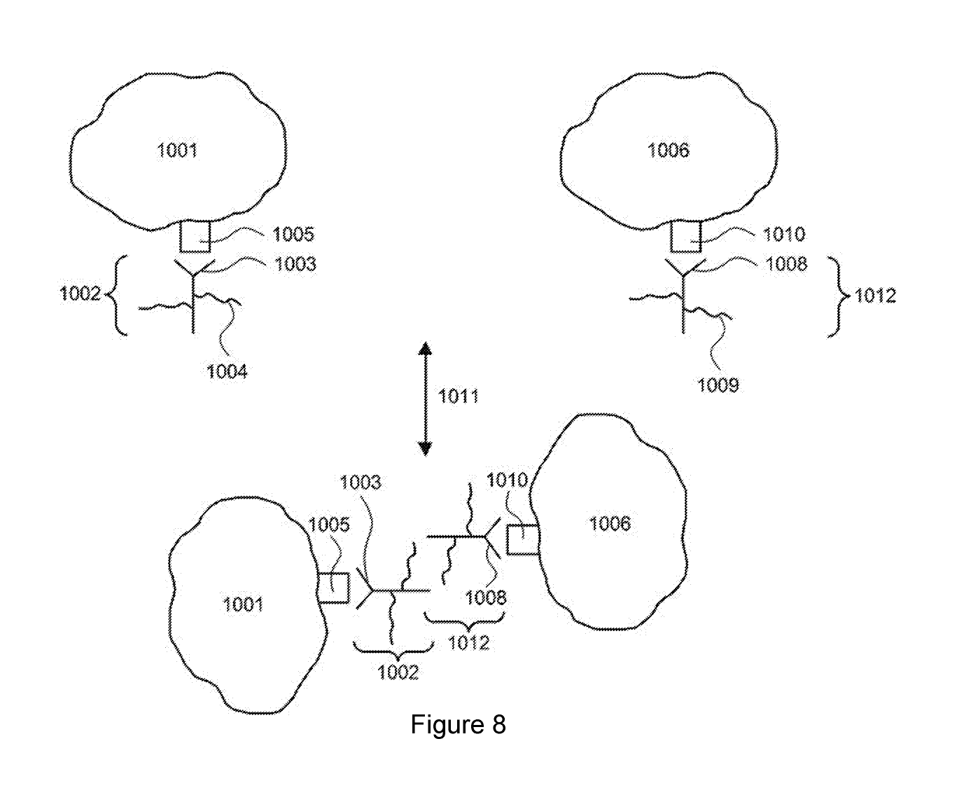

One embodiment of clustering cells is provided by FIG. 8. As is shown in the figure, the methods may include contacting a first cell 1001 with a plurality of binding entities, e.g., 1002 each comprising an affinity reagent 1003 bound to one or more polymers 1004 that are reversibly associative in response to a stimulus. In some versions, the first cell 1001 includes one or more cell surface molecules 1005 on its surface and contacting the first cell 1001 with the binding entities 1002 includes binding one or more of the affinity reagents 1003 to the one or more cell surface molecules 1005.

The methods also include contacting a second cell 1006 with a plurality of binding entities, e.g., 1012, each comprising an affinity reagent 1008 bound to one or more polymers 1009 that are reversibly associative in response to a stimulus. In some aspects, the second cell includes one or more cell surface molecules 1010 on its surface and contacting the second cell 1006 with the binding entities includes binding one or more of the affinity reagents 1008 to the one or more cell surface molecules 1010. In some aspects, the second cell 1006 may be the same or different type as the first cell 1001.

The methods also include applying a stimulus, e.g., arrow 1011, effective in associating at least one of the binding entities bound to the first cell 1001 and at least one of the binding entities bound to the second cell 1006 to one another and thereby co-localizing the first and second cells. In various embodiments, the affinity reagents 1003 bound to the first cell and the affinity reagents 1008 bound to the second cell may be of the same type or of different types. Such affinity reagents may be any type or combination of types of the affinity reagents described herein. Furthermore, the one or more cell surface molecules 1005 of the first cell may be of the same or different types than the one or more cell surface molecules 1010 of the second cell. Such cell surface molecules may be any type or combination of types of the cell surface molecules described herein.

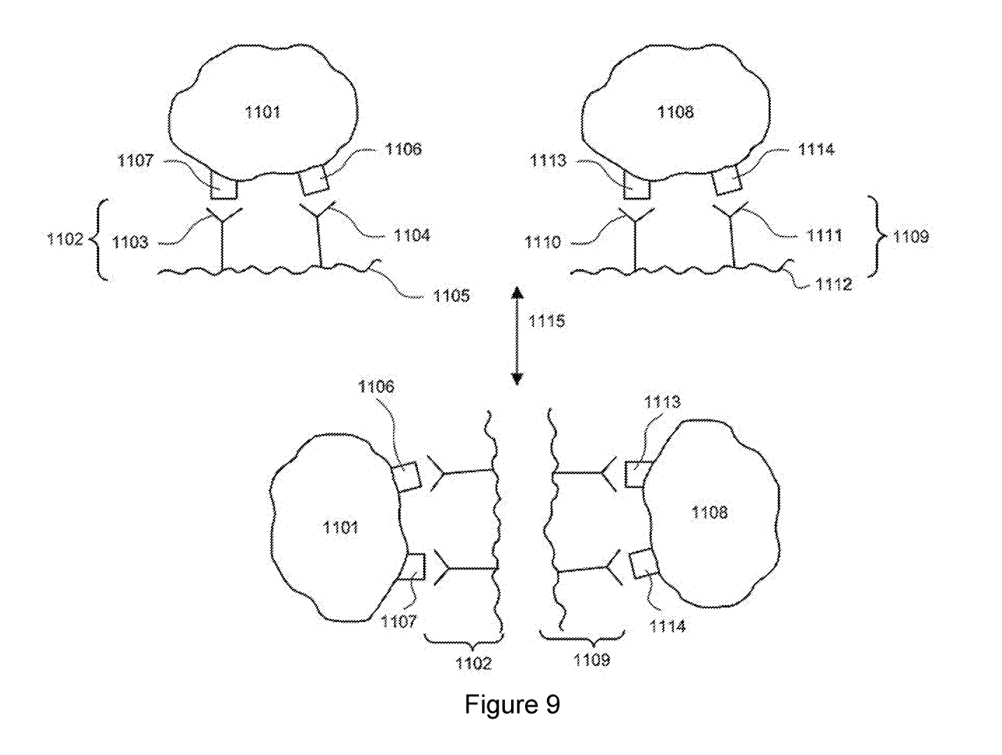

Another embodiment of clustering cells is illustrated in FIG. 9. Specifically, the subject methods may include contacting a first cell 1101 with a binding entity 1102 comprising a plurality of affinity reagents 1103, 1104 bound to one or more polymers 1105 that are reversibly associative in response to a stimulus. In some versions, the first cell includes cell surface molecules 1106, 1107, on its surface and contacting the first cell with the binding entity includes binding a plurality of the affinity reagents to the cell surface molecules.

According to some versions of the subject methods, the methods include contacting a second cell 1108 with a binding entity 1109 comprising a plurality of affinity reagents 1110, 1111, bound to one or more polymers 1112 that are reversibly associative in response to a stimulus. In some aspects, the second cell includes cell surface molecules 1113, 1114, on its surface and contacting the second cell with the binding entity includes binding a plurality of the affinity reagents to the cell surface molecules. In some aspects, the second cell 1108 is the same or different type than the first cell 1101.

The methods also include applying a stimulus, e.g., arrow 1115, effective in associating at least one of the binding entities bound to the first cell and at least one of the binding entities bound to the second cell to one another and thereby clustering the first cell 1101 and second cell 1108.

In various embodiments, the affinity reagents 1103, 1104 bound to the first cell and the affinity reagents 1110, 1111 bound to the second cell may be of the same type or of different types. Also, the affinity reagents 1103, 1104 bound to the first cell may be of the same type or of different types. The affinity reagents 1110, 1111 bound to the second cell may be of the same type or of different types. Such affinity reagents may be any type or combination of types of the affinity reagents described herein.

Furthermore, the one or more cell surface molecules 1106, 1107 of the first cell may be of the same or different types than the one or more cell surface molecules 1113, 1114 of the second cell. The one or more cell surface molecules 1106, 1107 of the first cell may be of the same or different types. The one or more cell surface molecules 1113, 1114 of the second cell may also be of the same or different types. Such cell surface molecules may be any type or combination of types of the cell surface molecules described herein.

In some versions, and as described further herein, the methods include optionally applying nanoparticles, such as magnetic nanoparticles (mNPs) in association with the disclosed polymers. Methods of making such nanoparticles are also provided. Methods of applying and making nanoparticles which may be used in accordance with the subject embodiments are described in WO 2014/194102 (PCT/US2014/040038), the disclosure of which is incorporated by reference herein in its entirety for all purposes.

A polymer-Ab or polymer-ligand conjugate (and further conjugates, such as the magnetic nanoparticles described herein) can be in a dried form and added to the cell suspension fluid or solvated in a solution added to the cell suspension fluid. In some versions, refrigeration of a solution containing solvated binding entity or magnetic nanoparticle is not required.

Various embodiments include methods of concentrating and/or isolating a target cell in a liquid, including applying a magnetic field to an aggregate in the liquid to provide a collected aggregate by magnetophoresis, wherein the aggregate includes: (a) a stimuli-responsive magnetic nanoparticle including a first stimuli-responsive polymer attached to a magnetic core, wherein the stimuli-responsive magnetic nanoparticle does not include a binding entity; and (b) a stimuli-responsive binding entity, e.g., polymer-affinity reagent conjugate, includes a second stimuli-responsive polymer attached to an affinity reagent, wherein the affinity reagent is capable of binding to a target, e.g., a cell surface receptor; wherein the aggregate is formed through associative interaction between the first stimuli-responsive polymer and the second stimuli-responsive polymer. In some aspects, a magnetic field does not induce magnetophoresis in a non-aggregated stimuli-responsive magnetic nanoparticle in the liquid. In various aspects, a method includes a step of concentrating the aggregate in the liquid. In various aspects, a method includes a step of isolating the aggregate with a magnetic field in the liquid. According to some embodiments, the first stimuli-responsive polymer and the second stimuli-responsive polymer are responsive to stimuli such as: temperature, pH, light, photo-irradiation, specific anions and/or cations, exposure to an electric field, ionic strength, or any combinations thereof.

In various embodiments, the subject disclosure includes methods of clustering initially distanced cell surface molecules. Such methods can include contacting a cell with a plurality of first binding entities each including, for example, an affinity reagent bound to one or more first polymers that are reversibly associative in response to a first stimulus. The methods can also include contacting a cell with a first binding entity including a plurality of affinity reagents bound to a polymer that is reversibly associative in response to a first stimulus. In some versions, a cell includes cell surface molecules on its surface and contacting the cell with the first binding entities includes binding a plurality of the affinity reagents to the cell surface molecules. The methods also can include applying a first stimulus effective in associating at least some of the plurality of first binding entities to one another and thereby clustering cell surface molecules bound to affinity reagents. In some versions, the methods include applying a first stimulus effective in associating at least some of the plurality of affinity reagents, such as affinity reagents bound to a binding entity, to one another and thereby clustering cell surface molecules bound to affinity reagents.

Methods as provided herein can further include contacting a cell with a plurality of second binding entities each including an affinity reagent bound to one or more second polymers that are reversibly associative in response to a second stimulus. The methods can also include contacting a cell with a second binding entity including a plurality of affinity reagents bound to a polymer that is reversibly associative in response to a second stimulus. In some versions, the cell includes cell surface molecules on its surface and contacting the cell with the second binding entities includes binding a plurality of the affinity reagents to the cell surface molecules. The methods further include applying a second stimulus different than the first stimulus and effective in associating at least some of the plurality of second binding entities to one another and thereby clustering cell surface molecules bound to affinity reagents. Also, in some versions, the methods include applying a second stimulus different than the first stimulus and effective in associating at least some of the plurality of affinity reagents, such as affinity reagents bound to a binding entity, to one another and thereby clustering cell surface molecules bound to affinity reagents.

Methods as provided herein can further include contacting a cell with a plurality of third, fourth, fifth, sixth, etc., binding entities each including an affinity reagent bound to one or more second polymers that are reversibly associative in response to a third, fourth, fifth, sixth, etc., stimulus different than any other applied stimulus. The methods further include applying a third, fourth, fifth, sixth, etc., stimulus different than other applied stimuli and effective in associating at least some of the plurality of third, fourth, fifth, sixth, etc., binding entities to one another and thereby clustering cell surface molecules bound to affinity reagents.

In various embodiments, a first stimulus as provided above is a change in temperature across a first temperature threshold, e.g., 18° C., and/or a second stimulus is a change in temperature across a second temperature threshold, e.g., 26° C., different than the first and/or a third stimulus is a change in temperature across a third temperature threshold, e.g., 32° C., different than the first and second thresholds. In some aspects, the third temperature threshold is higher than the first and second temperature thresholds. In various embodiments, a first temperature threshold is within a range of, for example, 15-25° C., such as 16-20° C., such as 17-19° C. In some embodiments, a second temperature threshold is within a range of, for example, 20-30° C., such as 22-28° C., such as 25-27° C. In various embodiments, a third temperature threshold is within a range of, for example, 25-40° C., such as 30-35° C., such as 31-33° C.

In addition, a first, second, third, fourth, fifth, and/or sixth, etc., stimulus as provided herein can be a change, such as a change across a threshold, in a condition selected from a group including temperature, pH, light, photo-irradiation, exposure to an electric field, ionic strength, specific anions, specific cations, or any combinations thereof.

The methods as provided herein also include methods of isolating target molecules. Target molecules are molecules which bind to affinity reagents or ligands. Target molecules can be any of the same types of molecules as cell surface molecules as provided herein. In various embodiments, target molecules are monoclonal antibodies such as IgG. Also, as provided according to the subject disclosure, affinity reagents can be protein A. One illustration the subject methods is provided, for example, by Example 9.

In some embodiments, the methods include contacting a binding entity comprising a plurality of affinity reagents bound to a polymer that is reversibly associative in response to a stimulus, such as any of the stimuli described herein, with a plurality of target molecules, and thereby binding the plurality of affinity reagents to the target molecules, wherein the binding is in a solution. A solution as provided herein can be a mixture of completely soluble elements, or a mixture of soluble and insoluble elements, or a transition between those states. A solution as provided herein can be a mixture of completely soluble elements, or a mixture of soluble and insoluble elements. In various embodiments, a solution is composed entirely of soluble elements. A solution can include one or more buffers and/or water. In various aspects, a solution can have any of the entities described herein suspended therein. A solution can also be substantially or completely devoid of any of the entities described herein. In various embodiments, binding entities or components thereof, e.g., polymers, are soluble in a first state and/or insoluble in a second state in response to a change in a stimulus. In some versions, the methods include separating insoluble elements, binding entities or components thereof, e.g., polymers, from other components, e.g., soluble components and/or buffer and/or water, of a solution.

The methods can also include applying the stimulus to associate at least some of the plurality of affinity reagents to one another and thereby clustering the target molecules bound to affinity reagents. The methods can also include contacting a magnetic nanoparticle (mNP) bound to a polymer that is reversibly associative in response to a stimulus, such as any of the stimuli described herein, with a plurality of target molecules and binding entities. Such contacting can include attaching the mNP and the contacted target molecules and/or binding entities to form a bound entity. As provided below, the bound entity can in turn be retained in a position, such as retained at a position within a column, or by applying a magnetic field thereto.

Also provided herein is a step of separating the binding entity from the solution. In some embodiments, separating the binding entity from the solution includes centrifuging the binding entity and the solution. In some versions, separating the binding entity from the solution includes passing the solution through a column and retaining the binding entity within the column. In yet other versions, separating the binding entity from the solution includes applying a magnetic field to the solution. In such embodiments, the binding entity or entities can be magnetic and/or be bound to a mNP. Also, in some instances, the methods also include isolating the target molecules from the binding entity.

In addition, in some aspects, the methods as provided herein include contacting a plurality of binding entities each comprising an affinity reagent bound to one or more polymers that are reversibly associative in response to a stimulus with a plurality of target molecules, and thereby binding the affinity reagents to the target molecules, wherein the binding is in a solution. In some aspects, the methods include applying the stimulus to associate at least some of the plurality of binding entities to one another and thereby clustering the target molecules bound to affinity reagents.

The methods can also include separating the binding entities from the solution such as, for example, centrifuging the binding entities and the solution to create one or more pellet including the binding entities. The pellets can then be removed from the mixture. Separation of binding entities from the solution can also include passing the solution through a column and retaining the binding entities within the column. Separation of binding entities from the solution can also include applying a magnetic field to the solution. In some instances, the methods also include isolating the target molecules from the binding entities.

### III. COMPOSITIONS

Stimuli-responsive reagents are provided wherein the reagents include one or more binding entities composed of one or more, such as a plurality of, affinity reagents, or ligands bound to one or more polymers that are reversibly associative in response to a stimulus.

A. Stimuli-Responsive Polymers & Affinity Reagents

The disclosed subject matter includes polymer-affinity reagent conjugates, also referred to herein as binding entities, that can comprise one or more stimuli-responsive polymers conjugated (e.g., covalently) to one or more affinity reagents or ligands. Binding entities may be configured to bind, for example, to one or more cell surface molecules, such as receptors. A stimuli-responsive polymer according to the subject embodiments is reversibly self-associative in response to a stimulus, or change in its environment. That is, stimuli-responsive polymers are hydrophilic and freely soluble in one condition and hydrophobic and self-associative in another condition.

A subject polymer can have a first state in which the polymer is not self-associative, and a second state in which the polymer is self-associative. The polymer adopts the second state in response to a stimulus, and reverts to the first state from the second state on reversal of the stimulus. In various embodiments, the polymer is a temperature-responsive polymer. In other embodiments, the polymer is a pH-responsive polymer. In yet other embodiments, the polymer is a light-responsive polymer. In yet other embodiments, the polymer is both temperature and pH-responsive. In yet other embodiments, the polymer is both temperature and ionic strength-responsive.

A stimulus can be a change in a condition such as temperature, pH, light, photo-irradiation, exposure to an electric field, ionic strength, or any combination thereof. As is described in greater detail below, the stimuli-responsive polymers can be homopolymers, copolymers, diblock copolymers, star polymers, brush polymers and/or graft copolymers. An example of a stimuli-responsive polymer that responds to changes in both temperature and ionic strength according to the disclosed embodiments is poly(N-isopropylacrylamide), or poly(NIPAM). Another example of a polymer according to the subject embodiments is poly(N-isopropylacrylamide-co-butyl acrylate).

In some versions of the subject disclosure, the polymers are smart polymers. “Smart” polymers are polymers that reversibly change their physical properties in response to small and controllable stimuli, e.g., changes in pH, temperature, and/or light, to control recognition events by acting as environmental antennae and switches. These smart polymers reversibly cycle between an extended and hydrophilic random coil, and a collapsed, hydrophobic state that is reduced in average volume by around 3-fold. The polymers can serve as environmental sensors and differentially control access of ligands or substrates to binding or catalytic sites as a function of their expanded or collapsed states. Such an approach can target mild environmental signals to specific conjugates, and thus, for example, allow differential control of different antibodies by using conjugated polymers that are sensitive to different signals, e.g., antibody 1 with pH, antibody 2 with temperature, antibody 3 with light. Smart polymers are also referred to as stimuli-responsive polymers or stimuli-sensitive polymers herein.

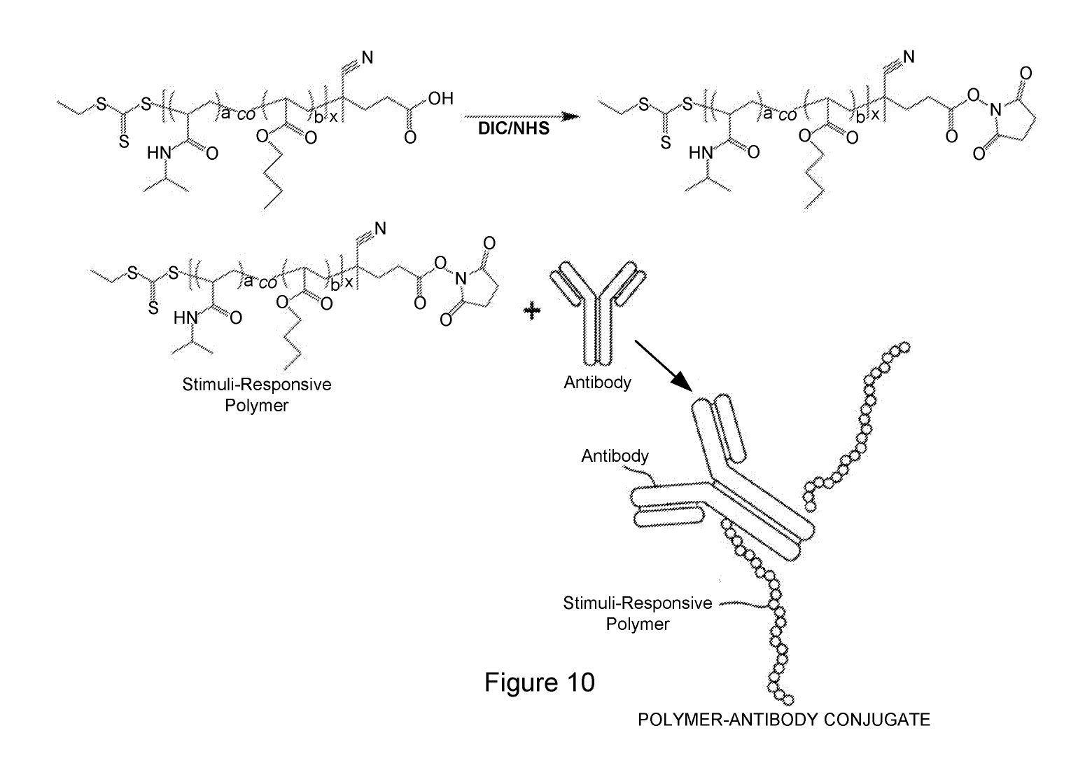

FIG. 10 illustrates one reaction scheme according to the subject embodiments for conjugating a polymer to an antibody in accordance with the embodiments provided herein. FIG. 10 specifically provides a chemical structure of a polymer according to the subject disclosure. Also shown is a reaction creating an amine-reactive polymer using DIC/NHS.

In various embodiments, a binding entity is a nanoparticle. In other embodiments, the binding entity is a microparticle. Binding entity size or a dimension thereof, such as length or diameter, can range, for example, from 5 to 5000 nm, such as from 50 to 1000 nm, such as from 100 to 2000 nm.

Affinity reagents as disclosed herein can include biomolecules and small molecules like biotin. Examples include monoclonal or polyclonal antibodies, antibody fragments, antigens, enzymes, streptavidin, protein A, cytokines, hormones, oligonucleotides, lipids, aptamers, polypeptide tags and even biologic particles such as viruses. A biomolecule can be a protein or a peptide, such as an enzyme, antibody, or affinity protein; a nucleic acid, such as a DNA or an RNA; a carbohydrate, such as a polysaccharide; or other biochemical or synthetic species.

The conjugation between the polymer and the affinity reagent can be mediated via a number of reactive groups. Examples include NHS esters and fluorophenol-based esters (for amine coupling), maleimides, thiols and pyridyl disulfides (for thiol coupling) and azides for click chemistry. The covalent linkage between the polymer and the affinity reagent can be stable or it may be reversible. An example of a stable covalent linkage is an amide bond. An example of a reversible covalent linkage is a disulfide bond.

In the presently disclosed subject matter, one or more stimuli-responsive polymers is conjugated, e.g., covalently conjugated to an affinity reagent, thus forming a binding entity. The stimuli-responsive behavior of the polymer is conferred to the affinity reagent. Therefore, under one condition, the polymer-affinity reagent conjugate is freely soluble and able to bind to its intended target/s. After applying a stimulus, the polymer becomes hydrophobic, causing co-aggregation of nearby polymer-affinity reagent conjugates. If the affinity reagent is a polyclonal or monoclonal Ab, the conjugate is referred to as a polymer-Ab conjugate. If the Ab is monoclonal anti-CD3, the conjugate is referred to as a polymer-anti-CD3 Ab conjugate. If the affinity reagent is a hormone or cytokine ligand, or other small molecule, for a cell receptor, it may be referred to as a polymer-ligand conjugate. Also, according to the subject embodiments, ligands, as set forth herein can be one or more DNA, RNA, pharmaceutical composition, and/or other small molecules which specifically bind cell, e.g., T cell and/or B cell, surface proteins.

In some versions of the embodiments, the reagents include a binding entity including a plurality, e.g., two, three, four, five or more, or ten or more, affinity reagents. In some versions, the plurality of affinity reagents includes a first affinity reagent and a second affinity reagent different than the first affinity reagent. As such, in some versions, each of the affinity reagents on a binding entity can be of the same type or they can be of a different type. Also, in some versions, the affinity reagents bind to cell surface molecules, e.g., receptors, when the binding entities are contacted to a cell including the cell surface molecules, e.g., receptors, and wherein applying the stimulus associates at least some of the plurality of affinity reagents to one another, such as by decreasing the distance between the cell surface molecules, e.g., receptors. For example, two or more cell surface molecules may have a first distance separating them, e.g., a distance along the surface of a cell membrane, and applying a stimulus may decrease the distance to a second distance which is smaller than the first distance. Cells, according to the subject embodiments can be cells of a subject such as cells of the adaptive immune system and/or may be antigen-presenting cells (APCs) such as dendritic cells, T cells and/or B cells.

As is shown, for example, in FIG. 4, the polymer-anti-CD3 Ab conjugates can be added to a human blood cell or purified T cell suspension according to the subject methods, allowing them to bind to CD3 cell surface receptors. A stimulus is applied, that causes co-aggregation of the polymer-Ab conjugates. The co-aggregation process brings the CD3 cell surface receptors in close proximity, enabling receptor cross-linking, and eventually leading to cell, e.g., T cell, activation and expansion.

The disclosed subject matter also includes the application of two or more different polymer affinity reagent conjugates, e.g., binding entities, which bind to different receptors. For example, one or many stimuli-responsive polymers can be conjugated to Ab #1 (e.g., anti-CD3 monoclonal Ab), and one or many stimuli-responsive polymers can be conjugated to Ab #2 (e.g., anti-CD28 monoclonal Ab). An exemplary illustration of such an embodiment is provided in FIG. 3. As is shown in FIG. 3, the two polymer-Ab conjugates can be added to the same cell suspension, allowing them to bind to, for example, CD3 and CD28 cell surface receptors. A stimulus is applied, that causes co-aggregation of the different polymer-Ab conjugates. The co-aggregation process brings the CD3 and CD28 cell surface receptors in close proximity, causing receptor cross-linking, formation of an immunologic synapse and/or T cell activation and/or expansion.

As is provided in the Examples section below, the disclosed subject matter also includes compositions and methods for using two different polymer-affinity reagent conjugates to activate T cells ex vivo.

Embodiments of the disclosed compositions include a stimuli-responsive polymer that is not conjugated to an affinity reagent. This ‘free’ stimuli-responsive polymer can be the same or different composition than the stimuli-responsive polymer conjugated to the affinity reagent but it does respond to the same stimulus. The ‘free’ stimuli-responsive polymer can be added to the cell suspension with the binding entities. The stimuli-responsive polymer may facilitate easier co-aggregation by acting as a bridge between closely associated, but not adjacent, polymer-affinity reagent conjugates during the co-aggregation process. Addition of supplemental stimuli-responsive polymer can also help co-aggregate polymer-affinity reagent conjugates whose cognate receptors are sparsely expressed on the cell surface.

Various embodiments of the compositions include one or more binding entities including a plurality of, e.g., 2, 3, 4, 5 or more, or 10 more, affinity reagents bound to a polymer, e.g., a single polymer, that is reversibly associative in response to a stimulus. One example of a polymer binding entity system that includes multivalent Ab loading onto single stimuli-responsive polymer molecules is provided by FIG. 5. The provided aspects may be employed in association with the stimuli-responsive polymers, affinity reagents and conjugation chemistries as described herein, such as in association with the monovalent polymer-affinity reagent system.

A stimuli-responsive polymer can contain multiple conjugation sites having the same or different reactive chemistries, which allows control over the number and type of affinity reagents conjugated to the stimuli-responsive polymer. For example, a stimuli-responsive polymer may contain multiple NHS esters for amine conjugations, or multiple maleimides for thiol conjugations. Alternatively, the polymer may contain both NHS esters and maleimides. The stimuli-responsive polymer may also contain protected reactive groups that can be de-protected for multi-step conjugation reactions. The stimuli-responsive polymer may be large, such as from ˜1,000-100,000 Da, 10,000-50,000 Da, or 5,000-30,000 Da, each inclusive, in molecular weight. As used herein, “inclusive” refers to a provided range including each of the listed numbers. Unless noted otherwise herein, all provided ranges are inclusive. In one embodiment, a single stimuli-responsive polymer acts as a backbone onto which more than one affinity reagent is covalently conjugated. For example, more than one anti-CD3 Ab may be conjugated to the stimuli-responsive polymer backbone. As is shown, for example in FIG. 5, this multivalent conjugate may be added to a cell suspension in its soluble state, in which it can bind to one or more cell surface molecules. More than one Ab can bind to the cell surface molecules and thereby effectively cross-link cell surface molecules. Also, in some versions, one or more Abs on the polymer backbone are not bound to the cell.

A stimulus may be applied, e.g., an increase in temperature, which causes the polymer backbone to aggregate. During the aggregation process, the stimuli-responsive polymer condenses on itself, which effectively shrinks the polymer and brings the Ab-bound cell surface receptors in close proximity. Such a change in conformation allows cell surface receptor clustering, co-localization and/or cross-linking, leading to cell, e.g., T cell, activation and expansion or other downstream signal transduction.

In the example shown in FIG. 5, the affinity reagents are the same, but they also may be different. For example, as is provided in FIG. 6, both anti-CD3 Abs and anti-CD28 Abs can be conjugated to a single stimuli-responsive polymer. In such an embodiment, multiple anti-CD3 and anti-CD28 Abs are first conjugated to a single stimuli-responsive polymer to form a multivalent stimuli-responsive polymer-Ab conjugate. As is shown, for example in FIG. 6, a multivalent conjugate is added to a cell suspension in its soluble state, in which it can bind to one or more cell surface molecules. Then a stimulus, such as an increase in temperature, is applied. Such a stimulus causes the polymer backbone to aggregate. During the aggregation process, the stimuli-responsive polymer condenses on itself, effectively shrinking and bringing the Ab-bound cell surface molecules in close proximity. Such a conformation change causes cell surface molecule clustering, co-localization and/or cross-linking, resulting in cell, e.g., T cell, activation and expansion.

As noted above, the presently disclosed subject matter may employ a stimuli-responsive polymer conjugate. The conjugate includes a polymer covalently coupled to an affinity reagent, wherein the polymer is reversibly self-associative in response to a stimulus. The polymer has a first state in which the polymer is not self-associative, and a second state in which the polymer is self-associative. The polymer adopts the second state in response to a stimulus, and reverts to the first state from the second state on removal of the stimulus. The stimuli-responsive polymer imparts stimuli responsiveness to the conjugate.

The stimuli-response polymer can be any one of a variety of polymers that change their associative properties (e.g., change from hydrophilic to hydrophobic) in response to a stimulus (e. g., temperature, pH, wavelength of light, ion concentration). The stimuli-responsive polymers are synthetic or natural polymers that exhibit reversible conformational or physico-chemical changes such as folding/unfolding transitions, reversible precipitation behavior, or other conformational changes in response to changes in temperature, light, pH, ions, or pressure. Representative stimuli-responsive polymers include temperature-sensitive polymers, pH-sensitive polymers, and light-sensitive polymers.

Stimulus-responsive polymers useful in making the conjugates and materials described herein can be any which are sensitive to a stimulus that cause significant conformational changes in the polymer. Illustrative polymers described herein include temperature-, pH-, ion- and/or light-sensitive polymers. Hoffman, A. S., “Intelligent Polymers in Medicine and Biotechnology,” Artif Organs. 19:458-467 (1995); Chen, G. H. and A. S. Hoffman, “A New Temperature- and Ph-Responsive Copolymer for Possible Use in Protein Conjugation,” Macromol. Chem. Phys. 196:1251-1259 (1995); Irie, M. and D. Kungwatchakun, “Photoresponsive Polymers. Mechanochemistry of Polyacrylamide Gels Having Triphenylmethane Leuco Derivatives,” Makromol. Chem., Rapid Commun 5:829-832 (1985); and Irie, M., “Light-induced Reversible Conformational Changes of Polymers in Solution and Gel Phase,” ACS Polym. Preprints, 27(2):342-343 (1986); which are incorporated by reference herein.

Stimuli-responsive oligomers and polymers useful in the conjugates and materials described herein can be synthesized that range in molecular weight from about 1,000 to 100,000 Daltons. In one embodiment, these syntheses are based on the chain transfer-initiated free radical polymerization of vinyl-type monomers, as described herein, and by (1) Tanaka, T., “Gels,” Sci. Amer 244:124-138 (1981); 2) Osada, Y. and S. B. Ross-Murphy, “Intelligent Gels,” Sci. Amer, 268:82-87 (1993); (3) Hoffman, A. S., “Intelligent Polymers in Medicine and Biotechnology,” Artif. Organs 19:458-467 (1995); also Macromol. Symp. 98:645-664 (1995); (4) Feijen, J., I. Feil, F. J. van der Gaag, Y. H. Bae and S. W. Kim, “Thermosensitive Polymers and Hydrogels Based on N-isopropylacrylamide,” 11th European Conf. on Biomtls: 256-260 (1994); (5) Monji, N. and A. S. Hoffman, “A Novel Immunoassay System and Bioseparation Process Based on Thermal Phase Separating Polymers,” Appl. Biochem. and Biotech. 14:107-120 (1987); (6) Fujimura, M., T. Mori and T. Tosa, “Preparation and Properties of Soluble-Insoluble Immobilized Proteases,” Biotech. Bioeng. 29:747-752 (1987); (7) Nguyen, A. L. and J. H. T. Luong, “Synthesis and Applications of Water-Soluble Reactive Polymers for Purification and Immobilization of Biomolecules,” Biotech. Bioeng. 34:1186-1190 (1989); (8) Taniguchi, M., M. Kobayahi and M. Fujii, “Properties of a Reversible Soluble-Insoluble Cellulase and Its Application to Repeated Hydrolysis of Crystalline Cellulose,” Biotech. Bioeng. 34:1092-1097 (1989); (9) Monji, N., C-A. Cole, M. Tam, L. Goldstein, R. C. Nowinski and A. S. Hoffman, “Application of a Thermally-Reversible Polymer-Antibody Conjugate in a Novel Membrane-Based Immunoassay,” Biochem. and Biophys. Res. Comm. 172:652-660 (1990); (10) Monji, N. C. A. Cole, and A. S. Hoffman, “Activated, N-Substituted Acrylamide Polymers for Antibody Coupling: Application to a Novel Membrane-Based Immunoassay,” J. Biomtls. Sci. Polymer Ed. 5:407-420 (1994); (11) Chen, J. P. and A. S. Hoffman, “Polymer-Protein Conjugates: Affinity Precipitation of Human IgG by Poly(N-Isopropyl Acrylamide)-Protein A Conjugates,” Biomtls. 11:631-634 (1990); (12) Park, T. G. and A. S. Hoffman, “Synthesis and Characterization of a Soluble, Temperature-Sensitive Polymer-Conjugated Enzyme, J. Biomtls. Sci. Polymer Ed. 4:493-504 (1993); (13) Chen, G. H., and A. S. Hoffman, Preparation and Properties of Thermo-Reversible, Phase-Separating Enzyme-Oligo(NIPAAm) Conjugates,” Bioconj. Chem. 4:509-514 (1993); (14) Ding, Z. L., G. H. Chen, and A. S. Hoffman, “Synthesis and Purification of Thermally-Sensitive Oligomer-Enzyme Conjugates of Poly (NIPAAm)-Trypsin,” Bioconj. Chem. 7:121-125 (1995); (15) Chen, G. H. and A. S. Hoffman, “A New Temperature- and pH-Responsive Copolymer for Possible Use in Protein Conjugation,” Macromol. Chem. Phys. 196:1251-1259 (1995); (16) Takei, Y. G., T. Aoki, K. Sanui, N. Ogata, T. Okano, and Y. Sakurai, “Temperature-responsive Bioconjugates. 1. Synthesis of Temperature-Responsive Oligomers with Reactive End Groups and their Coupling to Biomolecules,” Bioconj. Chem., 4, 42-46 (1993); (17) Takei, Y G., T. Aoki, K. Sanui, N. Ogata, T. Okano and Y. Sakurai, “Temperature-responsive Bioconjugates. 2. Molecular Design for Temperature-modulated Bioseparations,” Bioconj. Chem., 4, 341-346 (1993); (18) Takei, Y G., M. Matsukata, T. Aoki, K. Sanui, N. Ogata, A. Kikuchi, Y. Sakurai and T. Okano, “Temperature-responsive Bioconjugates. 3. Antibody-Poly(N-isopropylacrylamide) Conjugates for Temperature-Modulated Precipitations and Affinity Bioseparations,” Bioconj. Chem., 5, 577-582 (1994); (19) Matsukata, M.,Y. Takei, T. Aoki, K. Sanui, N. Ogata, Y. Sakurai and T. Okano, “Temperature Modulated Solubility-Activity Alterations for Poly(N-Isopropylacrylamide)-Lipase Conjugates,” J. Biochem., 116, 682-686 (1994); (20) Chilkoti, A., G. H. Chen, P. S. Stayton and A. S. Hoffman, “Site-Specific Conjugation of a Temperature-Sensitive Polymer to a Genetically-Engineered Protein,” Bioconj. Chem. 5:504-507 (1994); and (21) Stayton, P. S., T. Shimoboji, C. Long, A. Chilkoti, G. Chen, J. M. Harris and A. S. Hoffman, “Control of Protein-Ligand Recognition Using a Stimuli-Responsive Polymer,” Nature 378:472-474 (1995).

These types of monomers allow the design of copolymer compositions that respond to a specific stimulus and, in some embodiments, to two or more stimuli. In addition, control of molecular weight (by control of reactant concentrations and reaction conditions), composition, structure (e.g., linear homopolymer, linear copolymer, block or graft copolymer, “comb” polymers and “star” polymers) and type and number of reactant end groups permit “tailoring” of the appropriate polymer for conjugation to a specific site on the biomolecule or particle.

The stimuli-responsive polymers useful in the materials and methods of the disclosed subject matter include homopolymers and copolymers having stimuli responsive behavior. Other suitable stimuli-responsive polymers include block and graft copolymers having one or more stimuli-responsive polymer components. A suitable stimuli-responsive block copolymer may include, for example, a temperature-sensitive polymer block. A suitable stimuli-responsive graft copolymer may include, for example, a pH-sensitive polymer backbone or pendant temperature-sensitive polymer components. Also, in some versions of the subject disclosure, a polymer does not include a hydrophobic block.

i. Temperature-Sensitive Polymers

Illustrative embodiments of the many different types of temperature-sensitive polymers that may be conjugated to affinity reagents and/or ligands are polymers and copolymers of N-isopropyl-acrylamide (NIPAM). Poly(NIPAM) is a thermally sensitive polymer that precipitates out of water at 32° C., which is its lower critical solution temperature (LCST), or cloud point (Heskins and Guillet, J. Macromol. Sci.-Chem. A2:1441-1455 (1968)). When poly(NIPAM) is copolymerized with more hydrophilic comonomers such as acrylamide, the LCST is raised. The opposite occurs when it is copolymerized with more hydrophobic comonomers, such as butyl acrylate. Copolymers of NIPAM with more hydrophilic monomers, such as acrylamide, have a higher LCST, and a broader temperature range of precipitation, while copolymers with more hydrophobic monomers, such as butyl acrylate, have a lower LCST and usually are more likely to retain the sharp transition characteristic of poly(NIPAM) (Taylor and Cerankowski, J. Polymer Sci. 13:2551-2570 (1975); Priest et al., ACS Symposium Series 350:255-264 (1987); and Heskins and Guillet, J. Macromol. Sci.-Chem. A2:1441-1455 (1968), the disclosures of which are incorporated herein). Copolymers can be produced having higher or lower LCSTs and a broader temperature range of precipitation. Polymers and copolymers of N-isopropylacrylamide of the present disclosure can have varying proportions of hydrophilic and hydrophobic comonomers, with or without micelle-forming groups at the proximal terminus, or at both the proximal and distal termini.

Stimuli-responsive polymers such as poly(NIPAM) have been conjugated randomly to affinity molecules, such as monoclonal antibodies, for example, as described in U.S. Pat. No. 4,780,409; U.S. Pat. No. 9,080,933; and Monji and Hoffman, Appl. Biochem. Biotechnol. 14:107-120 (1987); Roy and Stayton, ACS Macro Letters 2:132-136 (2013). Activated groups (e.g., for conjugating to proteins), were formed randomly along the backbone or at the end group of poly(NIPAM) and were conjugated randomly to lysine amino groups on a monoclonal antibody and the conjugate was then applied in a temperature-induced phase-separation immunoassay. Activated poly(NIPAM) has also been conjugated by Hoffman and coworkers to protein A, various enzymes, biotin, phospholipids, RGD peptide sequences, and other interactive molecules. The random polymer-interactive molecular conjugates have been used in a variety of applications based on the thermally-induced phase separation step (Chen and Hoffman, Biomaterials 11:631-634 (1990); Miura et al., Abstr. 17th Ann. Meet. Soc. Biomaterials (1991); Wu et al., Polymer 33:4659-4662 (1992); Chen and Hoffman, Bioconjugate Chem. 4:509-514 (1993); Morris et al., J. Anal. Biochem. 41:991-997 (1993); Park and Hoffman, J. Biomaterials Sci. Polymer Ed. 4:493-504 (1993); Chen and Hoffman, J. Biomaterials Sci. Polymer Ed. 5:371-382 (1994)). Others have also randomly conjugated proteins to poly(NIPAM) (Nguyen and Luong, Biotech. Bioeng. 34:1186-1190 (1989); Takei et al., Bioconj. Chem. 4:42-46 (1993)) and to pH-sensitive polymers (Fujimura et al., supra.)). Most of these polymer-protein conjugates involved random lysine amino groups of proteins bound to the polymer through random activated groups pendant along the polymer backbone. More recently, a method based on chain transfer initiation polymerization has been used which yields relatively low MW polymers (called oligomers) usually with only one reactive end group (but the method may be adapted to synthesis of oligomers with a reactive group at each end) (Otsu, T., et al., Eur. Polym. J. 28:1325-1329, (1992)). (Chen and Hoffman, 1993, supra; Chen and Hoffman, 1994, supra, and Takei et al., supra).

The synthesis of an amino-terminated polymer proceeds by the radical polymerization of NIPAM in the presence of AIBN as an initiator and 1-aminoethanethiol-hydrochloride as a chain transfer reagent. To synthesize a chain with —COOH or —OH terminal groups, carboxyl- or hydroxyl-thiol chain transfer agents, respectively, have been used instead of the amino-thiol. It should be noted that the synthesis of the end-reactive polymers is based on a chain transfer initiation and termination mechanism. This yields a polymer chain, having a molecular weight ranging from 1000 to 1,000,000, such as 1000 to 500,000, such as 1000 to 200,000 Daltons, or 100,000 Daltons or greater. The shortest chains, 10,000 Daltons or less in molecular weight, are called “oligomers”. Oligomers of different molecular weights can be synthesized by simply changing the ratio of monomer to chain transfer reagent, and controlling their concentration levels, along with that of the initiator.

Polymers and/or oligomers of NIPAM (or other vinyl monomers) having a reactive group at one end are prepared by the radical co-polymerization of NIPAM and butyl acrylate using ACVA as initiator, plus a chain transfer agent with a “non-reactive” (e.g., C₂H₅) group at one end and the desired “reactive” group (e.g., —OH, —COOH, —NH₂) at the other end. The molecular weight of vinyl-type homopolymers and copolymers like these can be controlled by varying the concentration of the key reactants and the polymerization conditions. Chen and Hoffman, Bioconjugate Chem. 4:509-514 (1993) and Chen and Hoffman, J. Biomaterials Sci. Polymer Ed. 5:371-382 (1994), each of which is incorporated herein by reference. Appropriate quantities of NIPAM, butyl acrylate, ACVA and chain transfer reagent in DMF are placed in a thick-walled polymerization tube and the mixtures are degassed by bubbling with N₂ for 25 minutes or by freezing and evacuating and then thawing (4 times). After cooling for the last time, the tubes are evacuated and sealed prior to polymerization. The tubes are immersed in a water bath at 70° C. for 4 h. The resulting polymer is isolated by precipitation into diethyl ether and weighed to determine yield. The molecular weight of the polymer is determined either by titration (if the end group is amine or carboxyl) or gel permeation chromatography (GPC). In some embodiments, temperature sensitive oligopeptides also may be incorporated into the conjugates or nanoparticles.

The molecular weight of vinyl-type copolymers can be controlled by varying the concentration of the key reactants and the polymerization conditions. Since the amino-thiol chain transfer agent yields a broader molecular weight distribution than the hydroxyl or carboxylthiols (which may be undesirable), the carboxyl-terminated polymer can be synthesized and the —COOH end group converted to an amine group by activating with carbodiimide and coupling a diamine to the active ester group. Also, temperature sensitive oligopeptides can be incorporated into the conjugates.

ii. pH-Sensitive Polymers

Synthetic pH-sensitive polymers useful in making the conjugates described herein are typically based on pH-sensitive vinyl monomers, such as acrylic acid (AAc), methacrylic acid (MAAc) and other alkyl-substituted acrylic acids, maleic anhydride (MAnh), maleic acid (MAc), AMPS (2-Acrylamido-2-Methyl-1-Propanesulfonic Acid), N-vinyl formamide (NVA), N-vinyl acetamide (NVA) (the last two may be hydrolyzed to polyvinylamine after polymerization), aminoethyl methacrylate (AEMA), phosphorylethyl acrylate (PEA) or methacrylate (PEMA). pH-sensitive polymers may also be synthesized as polypeptides from amino acids (e.g., polylysine or polyglutamic acid) or derived from naturally-occurring polymers such as proteins (e. g., lysozyme, albumin, casein), or polysaccharides (e.g., alginic acid, hyaluronic acid, carrageenan, chitosan, carboxymethyl cellulose) or nucleic acids, such as DNA. pH-responsive polymers usually contain pendant pH-sensitive groups such as —OPO(OH)₂, —COOH or —NH₂ groups. With pH-responsive polymers, small changes in pH can stimulate phase separation, similar to the effect of temperature on solutions of poly(NIPAM) (Fujimura et al. Biotech. Bioeng. 29:747-752 (1987)). By randomly copolymerizing a thermally-sensitive NIPAM with a small amount (e.g., less than 10 mole percent) of a pH-sensitive comonomer such as AAc, a copolymer will display both temperature and pH sensitivity. Its LCST will be almost unaffected, sometimes even lowered a few degrees, at pH values where the comonomer is not ionized, but it will be dramatically raised if the pH-sensitive groups are ionized. When the pH-sensitive monomer is present in a higher content, the LCST response of the temperature sensitive component may be “eliminated” (e.g., no phase separation seen up to and above 100° C.). The pH-sensitive polymers of the present disclosure, in some embodiments, do not have micelle-forming groups at the proximal terminus, or at both the proximal and distal termini.

Graft and block copolymers of pH and temperature sensitive monomers can be synthesized which retain both pH and temperature transitions independently. Chen, G. H., and A. S. Hoffman, Nature 373:49-52 (1995). For example, a block copolymer having a pH-sensitive block (polyacrylic acid) and a temperature sensitive block (poly(NIPAM)) can be useful in the conjugates, materials, and methods of the disclosed subject matter. The graft and block copolymers having both pH and temperature sensitivity of the present disclosure may not have micelle-forming groups at the proximal terminus, or at both the proximal and distal termini.

iii. Light-Sensitive Polymers

Light-responsive polymers may contain chromophoric groups pendant to or along the main chain of the polymer and, when exposed to an appropriate wavelength of light, can be isomerized from the trans to the cis form, which is dipolar and more hydrophilic and can cause reversible polymer conformational changes. Other light sensitive compounds can also be converted by light stimulation from a relatively non-polar hydrophobic, non-ionized state to a hydrophilic, ionic state.

In the case of pendant light-sensitive group polymers, the light-sensitive dye, such as aromatic azo compounds or stilbene derivatives, may be conjugated to a reactive monomer (an exception is a dye such as chlorophyllin, which already has a vinyl group) and then homopolymerized or copolymerized with other conventional monomers, or copolymerized with temperature-sensitive or pH-sensitive monomers using the chain transfer polymerization as described above. The light sensitive group may also be conjugated to one end of a different (e.g., temperature) responsive polymer.

Although both pendant and main chain light sensitive polymers may be synthesized and are useful compositions for the methods and applications described herein, some light-sensitive polymers and copolymers thereof are typically synthesized from vinyl monomers that contain light-sensitive pendant groups. Copolymers of these types of monomers are prepared with “normal” water-soluble comonomers such as acrylamide, and also with temperature- or pH-sensitive comonomers such as NIPAM or AAc.

Light-sensitive compounds may be dye molecules that isomerize or become ionized when they absorb certain wavelengths of light, converting them from hydrophobic to hydrophilic conformations, or they may be other dye molecules which give off heat when they absorb certain wavelengths of light. In the former case, the isomerization alone can cause chain expansion or collapse, while in the latter case the polymer will precipitate only if it is also temperature-sensitive.

Light-responsive polymers usually contain chromophoric groups pendant to the main chain of the polymer. Typical chromophoric groups that have been used are the aromatic diazo dyes (Ciardelli, Biopolymers 23:1423-1437 (1984); Kungwatchakun and Irie, Makromol. Chem., Rapid Commun. 9:243-246 (1988); Lohmann and Petrak, CRC Crit. Rev. Therap. Drug Carrier Systems 5:263 (1989); Mamada et al., Macromolecules 23:1517 (1990), each of which is incorporated herein by reference). When this type of dye is exposed to 350-410 nm UV light, the trans form of the aromatic diazo dye, which is more hydrophobic, is isomerized to the cis form, which is dipolar and more hydrophilic, and this can cause polymer conformational changes, causing a turbid polymer solution to clear, depending on the degree of dye-conjugation to the backbone and the water solubility of the main unit of the backbone. Exposure to about 750 nm visible light will reverse the phenomenon. Such light-sensitive dyes may also be incorporated along the main chain of the backbone, such that the conformational changes due to light-induced isomerization of the dye will cause polymer chain conformational changes. Conversion of the pendant dye to a hydrophilic or hydrophobic state can also cause individual chains to expand or collapse their conformations. When the polymer main chain contains light sensitive groups (e.g. azo benzene dye) the light-stimulated state may actually contract and become more hydrophilic upon light-induced isomerization. The light-sensitive polymers can include polymers having pendant or backbone azobenzene groups.

iv. Specific Ion-Sensitive Polymers

Polysaccharides, such as carrageenan, that change their conformation, for example, from a random to an ordered conformation, as a function of exposure to specific ions, such as K⁺ or Ca⁺⁺, can also be used as the stimulus-responsive polymers. In another example, a solution of sodium alginate may be gelled by exposure to Ca++. Other specific ion-sensitive polymers include polymers with pendant ion chelating groups, such as histidine or EDTA.

v. Dual- or Multi-Sensitivity Polymers

If a light-sensitive polymer is also thermally-sensitive, the UV- or visible light-stimulated conversion of a chromophore conjugated along the backbone to a more hydrophobic or hydrophilic conformation can also stimulate the dissolution or precipitation of the copolymer, depending on the polymer composition and the temperature. If the dye absorbs the light and converts it to thermal energies rather than stimulating isomerization, then the localized heating can also stimulate a phase change in a temperature-sensitive polymer such as poly(NIPAM), when the system temperature is near the phase separation temperature. The ability to incorporate multiple sensitivities, such as temperature and light sensitivity, or temperature and pH sensitivity, along one backbone by vinyl monomer copolymerization lends great versatility to the synthesis and properties of the responsive polymer-protein conjugates. For example, dyes can be used which bind to protein recognition sites, and light-induced isomerization can cause loosening or detachment of the dye from the binding pocket (Bieth et al., Proc. Natl. Acad. Sci. USA 64:1103-1106 (1969)). This can be used for manipulating affinity processes by conjugating the dye to the free end of a temperature responsive polymer, such as ethylene oxide-propylene oxide (EO-PO) random copolymers available from Carbide. These polymers, —(CH₂CH₂O)ₓ—(CH₂CHCH₃O)_{y}—, have two reactive end groups. The phase separation point can be varied over a wide range, depending on the EO/PO ratio, and one end may be derivatized with the ligand dye and the other end with an —SH reactive group, such as vinyl sulfone (VS).

Conjugates according to the disclosed subject matter can include one or more chemical ligand (e.g., target binding partner). Such a chemical ligand may not be a biomolecule. Such a chemical ligand can also, for example, include one or more dye.

The conjugates of the disclosed subject matter can include a biomolecule (e.g., target binding partner). The biomolecule can be a protein or a peptide, such as an enzyme, antibody, or affinity protein; a nucleic acid oligomer, such as a DNA or an RNA; a carbohydrate, such as a polysaccharide; or other biochemical species. The biomolecule can have an active site, and the polymer can be covalently coupled to the biomolecule at a site proximate to the active site such that, when the polymer is self-associative, the binding site is inaccessible. Alternatively, the polymer is covalently coupled to the biomolecule at a site away from the active site such that, when the polymer is self-associative, the binding site is accessible.

The term “biomolecule” as used herein includes any molecule capable of a specific binding interaction with a target site, for example on a cell membrane, or on a molecule or atom. Thus, biomolecules include both ligands and receptors. A biomolecule may be a cell surface molecule.

The stimulus-responsive polymer can be conjugated to a variety of different biomolecules, including peptides, proteins, poly- or oligo-saccharides, glycoproteins, lipids and lipoproteins, and nucleic acids, as well as synthetic organic or inorganic molecules having a defined bioactivity, such as an antibiotic or anti-inflammatory agent, and which bind to a target site, for example, on a molecule such as a cell membrane receptor. Examples of protein biomolecules are ligand-binding proteins, including antibodies, lectins, hormones, cytokines, chemokines, receptors and enzymes. Other molecules which bind specifically or non-specifically to a target compound include poly- or oligosaccharides on glycoproteins which bind to receptors, for example, the carbohydrate on the ligand for the inflammatory mediators P-selectin and E-selectin, and nucleic acid sequences which bind to complementary sequences, such as ribozymes, antisense, external guide sequences for RNAase P, and aptamers.

The biomolecules can include a binding site, which may be the active site of an antibody or enzyme, the binding region of a receptor, or other functionally equivalent site. These sites are collectively referred to as the binding site.

The number of proteins whose interaction with specific binding partners can be controlled via site-specific conjugation of a stimulus-responsive polymer is large. These include, for example, antibodies (monoclonal, polyclonal, chimeric, single-chain or other recombinant forms), their protein/peptide antigens, protein/peptide hormones, cytokines, chemokines, streptavidin, avidin, protein A, protein G, growth factors and their respective receptors, DNA-binding proteins, cell membrane receptors, endosomal membrane receptors, nuclear membrane receptors, neural receptors, visual receptors, and muscle cell receptors. Oligonucleotides of any size include DNA (genomic or cDNA), RNA, antisense, ribozymes, and external guide sequences for RNAase P, and mimetics thereof which bind to cell receptors. Carbohydrates include tumor associated carbohydrates (e.g., Le^{x}, sialyl Le^{x}, Le^{y}, and others identified as tumor associated as described in U.S. Pat. No. 4,971,905, incorporated herein by reference), carbohydrates associated with cell adhesion receptors (e.g., Phillips et al., Science 250:1130-1132 (1990)), and other specific carbohydrate binding molecules, and carbohydrate mimetics thereof which bind to cell receptors.

Among the proteins, streptavidin is particularly useful as a model for other ligand-binding and cell or substrate-binding systems described herein. Streptavidin is an important component in many separations technologies that use the very strong association of the streptavidin-biotin affinity complex. (Wilchek and Bayer, Avidin-Biotin Technology, New York, Academic Press, Inc. (1990); and Green, Meth. Enzymol. 184:51-67. Protein G and protein A are proteins that bind IgG antibodies (Achari et al., Biochemistry 31:10449-10457 (1992), and Akerstrom and Bjorck, J. Biol. Chem. 261:10240-10247 (1986)) and are also useful as model systems. Representative immunoaffinity molecules include engineered single chain Fv antibody (Bird et al., Science 242:423-426 (1988) and U.S. Pat. No. 4,946,778 to Ladner et al.), incorporated herein by reference, Fab, Fab′, and monoclonal or polyclonal antibodies. Enzymes represent another important model system, as their activity can be turned on or off or modulated by the controlled collapse of the stimulus-responsive component at the active site.

In addition to their well-established uses in biotechnology, streptavidin, protein G, single-chain antibodies and enzymes are ideal model systems for several other important reasons. Genetic engineering systems for these proteins have been established, allowing convenient site-directed mutagenesis and the expression of large quantities of each protein in hosts such as E. coli. High-resolution crystal structures are available that provide a molecular “road map” of the ligand binding sites (Achari et al. supra; Hendrickson et al., Proc. Natl. Acad. Sci. USA 86:2190-2194 (1989); Weber et al., Science 243:85-88 (1992); Derrick and Wigley, Nature 359:752-754 (1992); Mian, J. Mol. Biol. 217:133-151 (1991)). This structural information provides a rational basis for the design of affinity or activity switch site-directed mutants. Of course, proteins which already have one, two or more cysteine residues located at a site convenient for attaching a stimulus-responsive component are ready for attachment of the stimulus-responsive component and need not have other cysteine residues engineered therein (unless another thiol group is desired in a specific site or unless reaction of the wild type —SH group undesirably changes the protein bioactivity). Other sites on the proteins can also be used, including amino acids substituted with non-natural amino acids.

Other affinity systems include concanavalin A, which has an affinity to sugars (e.g., mannose, glucose, and galactose).

Furthermore, the subject disclosure may include one or more aspects of long polymer and antibody-based structures termed ‘nanoworms’. Nanoworms include multiple antibody molecules conjugated to a long, ˜100-200 nm, polymeric backbone. One version of such a system uses ˜3-5 anti-CD3 Abs conjugated to a single, long polymer. This polymer can activate human T cells, as measured by proliferation, cell surface marker expression and release of cytokines (Mandal, et al., Chemical Science 4:4168-4174 (2015)). This flexible polymer may also exhibit better T cell responses than anti-CD3 conjugated microparticles.

B. Stimuli-Responsive Polymer Conjugate Aggregates

In one aspect of the disclosed subject matter, formations (or aggregates) made up of a plurality of polymer-biomolecule or polymer-ligand conjugates are provided. In the aggregate, each conjugate includes one or more polymers covalently coupled to one or more biomolecules or ligands, and the polymer is reversibly self-associative in response to a stimulus. In the aggregate, the plurality of conjugates is adhered through polymer association. The aggregate can be controllably formed to have an effective size from 50 to 5000 nm. In various embodiments, the aggregate is a nanoparticle. In other embodiments, the aggregate is a microparticle. Because the aggregate is controllably formed by the application of a stimulus to a stimuli-responsive polymer conjugate and through polymer association, the aggregate can be dissociated to its component conjugates by removal of the stimulus causing association.

C. Nanoparticles and Beads

The disclosed compositions can also optionally include stimuli-responsive nanoparticles such as magnetic nanoparticles. Such nanoparticles can be bound to polymers and/or one or more, such as a plurality of, affinity reagents. The stimuli-responsive magnetic nanoparticles can respond to the same stimulus as a polymer-affinity reagent conjugate. The stimuli-responsive magnetic nanoparticles may facilitate easier co-aggregation between closely associated, but not adjacent, polymer-affinity reagent conjugates. In the aggregated state, the polymer-affinity reagent—mNP aggregate can be separated with a magnetic field. With this magnetic nanoparticle system, cells can be positively or negatively selected and/or sorted according to the subject methods before or after an activation and/or expansion step. Such sorting may include moving the magnetic nanoparticles using a magnetic field such as by moving one or more magnets exerting a magnetic field on the nanoparticles.

In an aspect of the disclosed subject matter, a bead such as a modified bead is optionally provided. The bead includes a target binding partner and a polymer. The target binding partner is capable of forming an associative interaction with a target compound, and the polymer is reversibly associative in response to a stimulus. In various embodiments, each of the target binding partner and polymer is covalently coupled to the bead. In other embodiments, the bead further includes a second polymer reversibly responsive to a second stimulus and a second target binding partner that forms an associative interaction with a second target compound. In other embodiments, the bead includes a plurality of different target binding partners and a plurality of different polymers.

In one aspect, a stimuli-responsive reagent is provided. In an embodiment, the stimuli-responsive reagent includes a stimuli-responsive magnetic nanoparticle that includes a first stimuli-responsive polymer attached to a magnetic core; and a stimuli-responsive binding entity that includes a second stimuli-responsive polymer attached to a first affinity reagent, wherein the first affinity reagent is capable of binding to a target. In some versions, the aggregate is formed through associative interaction between the first stimuli-responsive polymer and the second stimuli-responsive polymer.

In some of the embodiments provided herein, magnets are used for magnetophoresis to manipulate (e.g., move or concentrate) magnetic aggregates in solution. The nature of the magnets described herein is not important, so long as the magnet produces a sufficient magnetic field to produce a force sufficient to move and concentrate the magnetic aggregates as necessary. In various embodiments, the magnets are permanent magnets, such as ceramic or neodymium-containing magnets. In various embodiments, the magnets are electromagnets. In various embodiments, the magnetic field has a strength of from 1 to 20 kilogauss.

Also, as used herein, the term magnetic nanoparticle (mNP) describes a particle of 500 nm or less in diameter, such as 250 nm in diameter or less, that will magnetophorese when in a solution and exposed to a magnetic field of sufficient strength. In various embodiments, the stimuli-responsive magnetic particles are composed of a first stimuli-responsive polymer attached to a magnetic core.

Suitable magnetic particles are particles that are responsive to a magnetic field and magnetophorese through a medium in response to the application of a magnetic field. Representative magnetic particles include particles that include a suitable metal or metal oxide. Suitable metals and metal oxides include iron, nickel, cobalt, iron platinum, zinc selenide, ferrous oxide, ferric oxide, cobalt oxide, aluminum oxide, germanium oxide, tin dioxide, titanium dioxide, gadolinium oxide, indium tin oxide, cobalt iron oxide, magnesium iron oxide, manganese iron oxide, and mixtures thereof.

In an embodiment, the magnetic particles are magnetic nanoparticles. In various embodiments, the magnetic nanoparticles have a largest dimension of from 1 nm to 500 nm, such as 5 nm to 250 nm, such as 5 nm to 150 nm.

In some versions, mNPs include a metal oxide core; and a shell that includes a stimuli-responsive polymer having a terminal group, or multiple pendant groups, that directly coordinates to the metal oxide core. The stimuli-responsive polymer may or may not include a micelle-forming group at least at a proximal terminus of the polymer, with respect to the metal oxide core. The stimuli-responsive polymers of the present disclosure may or may not be polymers without a micelle-forming group at a proximal polymer terminus to the metal oxide core of the mNP, when coordinated to the metal oxide core.

In some embodiments, the stimuli-responsive nanoparticle includes a core including a magnetic metal oxide formed from the metal cation. Non-limiting examples of magnetic metal oxide include, for example, iron oxide (e.g., ferric oxide, ferrous oxide), nickel oxide, nickel oxide, chromium oxide, gadolinium oxide, dysprosium oxide, and manganese oxide, or any combination thereof.

In some embodiments, the stimuli-responsive polymer is coordinated to the core via a terminal functional group or multiple pendant functional groups (e.g., carboxylate, primary amine, secondary amine, hydroxyl, an aldehyde, a ketone, an azide, and/or a hydrazide) on the stimuli-responsive polymer. The stimuli-responsive polymer does not include a group (e.g., an alkyl group, aryl group, a hydrophobic copolymer block, a polypeptide, etc.) that is capable of forming a micelle at the proximal polymer terminus to the metal oxide core, or at both the proximal and distal polymer termini to the metal oxide core. In some embodiments, the stimuli-responsive polymer has no micelle-forming group on any terminus. In some embodiments, the stimuli-responsive polymer has no micelle-forming group (e.g., no micelle-forming group on any terminus, as pendant groups, or on the polymer backbone).

In various embodiments, the magnetic nanoparticle-producing stimuli-responsive polymer includes polymers and copolymers of NIPAM, and the polymer and copolymers of NIPAM includes a terminus distal to the metal oxide core having a formula

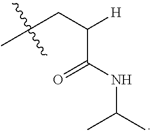 [SMILES: *CCC(=O)NC(C)C]

In some embodiments, the stimuli-responsive mNP includes a stimuli-responsive polymer to metal oxide mass ratio of from 0.5:1 to 20:1 (e.g., from 2:1 to 3:1, or from 1:1 to 2:1). In yet other embodiments, the stimuli-responsive mNP includes a stimuli-responsive polymer to metal oxide mass ratio of less than 1:1. For example, the polymer to metal oxide mass ratio can be 2:1. The mass ratio of polymer can be determined, for example, by thermogravimetric analysis, from the ratio of stimuli-responsive polymer decomposition mass loss and a remaining mass after polymer removal.

In some embodiments, the stimuli-responsive nanoparticle has a hydrodynamic diameter of from 1 nm to 500 nm, such as 10 nm to 250 nm, such as 10 nm (e.g., from 20 nm, from 30 nm, or from 40 nm) to 150 nm (e.g., to 40 nm, to 30 nm, or to 20 nm). For example, the stimuli-responsive nanoparticle can have a hydrodynamic diameter of from 10 nm to 35 nm (e.g., from 15 nm to 30 nm). The stimuli-responsive nanoparticle can respond to a stimulus such as temperature, pH, light, electric field, and/or ionic strength.

Also, in various embodiments, the subject disclosure includes both stimuli-responsive polymer conjugated to affinity reagents and stimuli-responsive magnetic nanoparticles which may be bound, e.g., covalently bound, to the polymers. Such an arrangement is referred to herein as the ‘binary reagent system.’ The binary reagent system may be applied to cross-link cell surface receptors. Such a system may also be applied to capture and to concentrate a target, such as one or more specific cells. Further details of the binary reagent system and aspects thereof are described, for example, in U.S. Pat. No. 9,080,933, which is incorporated by reference herein in its entirety. In addition, details of stimuli-responsive magnetic nanoparticles and aspects thereof are described, for example, in U.S. Pat. No. 7,981,688, and WO 2014/194102 (PCT/US2014/040038), which are incorporated by reference herein in their entirety.

In an embodiment, the magnetic nanoparticles are of a size and a composition such that a single magnetic nanoparticle will not affect magnetophoretic separation of an aggregate. Magnetophoretic separation is only effected using the magnetic nanoparticles when aggregated in aggregates including a plurality of magnetic nanoparticles. The aggregates of the disclosed subject matter, therefore, contain a plurality of magnetic nanoparticles, if present, and a plurality of binding entities, that had previously bound to their targets. The plurality of magnetic nanoparticles in the aggregates provides sufficient paramagnetism to enable magnetophoretic separation.

The magnetophoretic mobility of the aggregates governs the degree to which an aggregate will magnetophorese. The magnetic aggregate separation is influenced by many factors, including the number of individual magnetic particles in an aggregate, magnetic particle size, magnetic field strength, and solution viscosity. The magnetophoretic mobility needs to overcome diffusion before any magnetic separation will occur. For example, if a magnet with 32 MGa maximum energy product is used, the magnetophoretic mobility can overcome diffusion and control the particle movement when the aggregates reach a size of 50 nm or less, if iron oxide mNPs are used. Separation speed will improve with increased field strength, if all other characteristics of the system remain the same.

D. Polymer Capture

In some embodiments, the stimuli-responsive mNPs include polymers having distal (away from the mNP core) functional groups for covalently coupling an affinity reagent and/or a cell portion, e.g., a receptor or a capture molecule thereof. The terminal functional group on the stimuli-responsive polymer refers to any reactive group that may be derivatized to make it reactive with the affinity reagent, such as carboxyl, hydroxyl, and amine groups. The distal functional group may be derivatized to form reactive groups such as thiol, ketone, N-hydroxy succinimide esters, N-hydroxy maleimide esters, tetrafluorophenyl esters, pentafluorophenyl esters, carbonyl imidazoles, carbodiimide esters, vinyl sulfone, acrylate, benzyl halide, tosylate, tresylate, aldehyde, hydrazide, acid halide, p-nitrophenolic esters, and hydroperoxides. In one embodiment, the distal functional group on the stimuli-responsive polymer is a carboxylic group.

The distal functional group on the stimuli-responsive polymer can be coupled with an affinity reagent through covalent bonds, including but not limited to amide, ester, ether, thioether, disulfide, hydrazone, acetal, ketal, ketone, anhydride, urethane, urea, and carbamate bonds. In one embodiment, the biotin moiety is coupled to the stimuli-responsive polymer through an amide bond.

The distal functional group can be covalently coupled to an affinity reagent, such as a protein, a nucleic acid oligomer (DNA or RNA), an antibody, an antigen, an enzyme or an enzyme substrate. The affinity reagent can be further coupled with a target molecule, such as a protein, a nucleic acid oligomer (DNA or RNA), an antigen, an antibody, an enzyme, an enzyme substrate or a cell through covalent or non-covalent interaction. In one embodiment, the terminal functional group is coupled to a biotin, the affinity reagent, to afford a biotinylated nanoparticle. In one embodiment, the biotinylated nanoparticle can be further conjugated to a streptavidin, the target molecule, to yield a streptavidin-conjugated biotinylated nanoparticle that can be coupled to a biotinylated target molecule.

An affinity reagent and a target molecule form a binding pair. Each has an affinity toward the other (e.g., antigen and antibody). Each of the capture molecule and the target molecule can be a variety of different molecules, including peptides, proteins, poly- or oligosaccharides, glycoproteins, lipids and lipoproteins, and nucleic acids, as well as synthetic organic or inorganic molecules having a defined bioactivity, such as an antibiotic or anti-inflammatory agent, that binds to a target site, such as a cell membrane receptor. The exemplary proteins include antibodies (monoclonal, polyclonal, chimeric, single-chain or other recombinant forms), their protein/peptide antigens, protein/peptide hormones, streptavidin, avidin, protein A, protein G, cytokines or chemokines or growth factors and their respective receptors, DNA-binding proteins, cell membrane receptors, endosomal membrane receptors, nuclear membrane receptors, neuron receptors, visual receptors, and muscle cell receptors. Exemplary oligonucleotides include DNA (genomic or cDNA), RNA, antisense, ribozymes, and external guide sequences for RNAase P, and can range in size from short oligonucleotide primers up to entire genes. Carbohydrates include tumor associated carbohydrates (e.g., Le^{X}, sialyl Le^{X}, Le^{Y}, and others identified as tumor associated as described in U.S. Pat. No. 4,971,905, incorporated herein by reference), carbohydrates associated with cell adhesion receptors (e.g., Phillips et al., Science 250:1130-1132, 1990), and other specific carbohydrate binding molecules and mimetics thereof which are specific for cell membrane receptors or other synthetic species.

Among the proteins, streptavidin is particularly useful as a model for other binding entity-target molecule binding pair systems described herein. Streptavidin is an important component in many separations and diagnostic technologies which use the very strong association of the streptavidin-biotin affinity complex. (Wilchek and Bayer, Avidin-Biotin Technology, New York, Academic Press, Inc., 1990; and Green, Meth. Enzymol. 184:51-67). Protein G, a protein that binds IgG antibodies (Achari et al., Biochemistry 31:10449-10457, 1992, and Akerstrom and Bjorck, J Biol. Chem. 261:10240-10247, 1986) is also useful as a model system. Representative immunoaffinity molecules include engineered single chain Fv antibody (Bird et al., Science 242:423-426, 1988 and U.S. Pat. No. 4,946,778 to Ladner et al.), incorporated herein by reference, Fab, Fab′, and monoclonal or polyclonal antibodies.

According to one version, the affinity reagent is an antibody and the target molecule is an antigen. In another embodiment, both the affinity reagent and the target molecule are proteins. In another embodiment, the affinity reagent is a nucleic acid (DNA or RNA) and the target molecule is a complimentary nucleic acid (DNA or RNA). In another embodiment, the target molecule is a nucleic acid (DNA or RNA) and the affinity reagent is a protein. In another embodiment, the affinity reagent is a cell membrane receptor and the target molecule is a ligand. In another embodiment, the affinity reagent is a ligand and the target molecule is a cell membrane receptor. In another embodiment, the affinity reagent is an enzyme and the target molecule is a substrate. In another embodiment, the affinity reagent is biotin and the target molecule is streptavidin or avidin. In another embodiment, the target moiety is a cell (e.g., a living cell).

E. Assays and Methods that Utilize Stimuli-Responsive Polymers

The disclosure also provides assays and methods for using the stimuli-responsive polymer entities. In an embodiment, the disclosure provides an assay for manipulating molecules in solutions. This includes (a) contacting a target molecule, e.g., IgG, with a plurality of stimuli-responsive polymer-affinity reagent conjugates that have affinity toward the target; (b) optionally, contacting the target and conjugates with a plurality of stimuli-responsive mNPs or other surfaces that are modified with stimuli-responsive polymers, e.g., membranes; (c) aggregating the polymer-affinity reagent conjugates by applying an external stimulus; (d) further aggregating the polymer-affinity reagent conjugates by subjecting the aggregates to a physical separation method (e.g., application of a magnetic field, centrifugation); (e) regenerating the polymer-affinity regent conjugate aggregates by reversing the stimulus; (f) collecting or analyzing the target molecule that was captured by the polymer-affinity reagent conjugates.

In yet another embodiment, the disclosure provides an assay for detecting a diagnostic target, including: (a) contacting the diagnostic target with a plurality of stimuli-responsive mNPs, wherein each nanoparticle including an affinity reagent having affinity toward the target; (b) forming nanoparticle conjugates by combining the target with the stimuli-responsive mNPs; (c) aggregating the nanoparticle conjugates by applying an external stimulus; (d) further aggregating the nanoparticle conjugates by subjecting the aggregated nanoparticle conjugates to a magnetic field; (e) regenerating the nanoparticle conjugates by removing the stimulus and the magnetic field; and (f) analyzing the regenerated nanoparticles including the target.

In the above method, forming nanoparticle conjugates by combining the target with the stimuli-responsive mNPs provides a conjugate that includes a target bound to the affinity reagent. In the above method, regenerating the nanoparticle conjugates by removing the stimulus and the magnetic field provides released, free flowing nanoparticle conjugates in which the target is bound to the affinity reagent.

The regenerated nanoparticles including the target can be analyzed with or without release of the target from the nanoparticle.

The target can be a molecule that is indicative of a diseased condition or an indicator of exposure to a toxin, or a therapeutic drug that has been administered to a subject and whose concentration is to be monitored. The target can be any chemical, carbohydrate, virus, extracellular vesicle, protein, antibody, or nucleic acid. In one embodiment, the target is an antibody against hepatitis B virus. In one embodiment, the target is an antibody against hepatitis C virus. In one embodiment, the target molecule is an antibody against AIDS virus. In one embodiment, the target molecule is the malaria parasitic antigen, or the antiplasmodial antibodies, or the parasitic metabolic products, or the plasmodia nucleic acid fragments. In one embodiment, the target molecule is an antibody against tuberculosis bacteria. In one embodiment, the target molecule is a dengue fever virus or antibody.

Methods, devices, and assays that use mNPs are described, for example, in U.S. Pat. Nos. 8,507,283 and 7,981,688, and PCT/US2011/035256, the disclosures each of which are incorporated by reference herein in their entirety.

The subject disclosure also includes cell activation assays as described in other portions of this disclosure.

### IV. KITS

Also provided are kits that at least include one or more composition as provided herein. For example, a kit can include a first composition and a second composition, wherein the first and second compositions may be the same or different. A first and/or second composition can include a binding entity including an affinity reagent bound to one or more polymers that are reversibly associative in response to a stimulus. A first and/or second composition can also include a binding entity comprising a plurality of affinity reagents bound to a polymer that is reversibly associative in response to a stimulus. A kit also can include magnetic nanoparticles. Binding entities of the subject kits may have any feature or combination of features of the binding entities described herein. In some versions, the affinity reagent of the first composition is different than the affinity reagent of the second composition. Also in some versions, applying the stimulus associates the binding entity of the first composition to the binding entity of the second composition and thereby clusters cell surface molecules, e.g., receptors, bound to affinity reagents when the affinity reagents of the first composition and the affinity reagents of the second composition are bound to cell surface molecules, e.g., receptors.

Kits can also include one or more of the reagents described herein. The subject kits may also include one or more cells described herein, such as T cells or B cells. Such cells may be obtained from a subject or from the producer or creator of a cell line.

The subject kits can include two or more, e.g., a plurality, three, four, five, eight, ten, etc., compositions or other system aspects according to any of the embodiments described herein, or any combinations thereof. Kits may also include packaging, e.g., packaging for shipping the compositions without breaking or otherwise becoming unusable according to the subject methods.

In various embodiments, the kits include instructions, such as instructions for using the subject compositions or performing the subject methods. The instructions are, in some aspects, recorded on a suitable recording medium. For example, the instructions can be printed on a substrate, such as paper or plastic, etc. As such, the instructions can be present in the kits as a package insert, in the labeling of the container of the kit or components thereof, e.g., associated with the packaging or subpackaging, etc. In other embodiments, the instructions are present as an electronic storage data file present on a suitable computer readable storage medium, e.g., Portable Flash drive, CD-ROM, diskette, etc. The instructions can take any form, including complete instructions for how to use the compositions or as a website address with which instructions posted on the world wide web may be accessed.

### V. UTILITY

In various embodiments, the subject disclosure includes regulating cell signaling, in manners that act to, e.g., increase or decrease cell numbers or changing cell behaviors. Such regulation can be performed by clustering cell surface molecules, e.g., receptors and their ligands. Increasing and/or decreasing cell numbers, or change cell behavior, according to the subject methods, can be applied to make biological assays and/or healthcare treatments for one or more disease, e.g., cancer, more efficient and/or effective. The assays and/or treatments can be made more effective by reducing the complexity, time and/or cost of performing such procedures.

Also, in some commercial cell activation technologies (e.g., Dynabeads® CD3/CD28 CTS™ and Miltenyi Biotec MACS® GMP TransAct CD3/CD28 kit), the agonistic antibodies are covalently linked to magnetic particle surfaces. The attachment of the Abs to the particle surfaces presents several significant disadvantages. First, the sustained presence of magnetic particles bound to cells can reduce cell viability and functional responses. Second, if the magnetic particles are not removed after ex vivo cell expansion, the cell therapeutic product may face regulatory scrutiny in terms of residual particulates. Third, if the magnetic particles are removed, the bound and activated cells are likewise removed. Furthermore, after binding to cell surface receptors, the rigid particle surfaces may not permit receptor re-organization and cross-linking in an optimal fashion. Also, the initiating activation signal cannot be controlled or reversed with other commercial technologies for cell activation.

However, the stimuli-responsive polymer-affinity reagent conjugates disclosed herein do not require rigid magnetic particles to provide the signal for cell activation. As such, risks associated with particle toxicity or particle residuals in the final cell product are significantly reduced or eliminated. Also, the activation signal can be controlled via application of a stimulus according to the subject embodiments. Later reversal of the stimulus can remove the initial activation signal, which may help to modulate activation and subsequent cell expansion properties.

In addition, T cell isolation, activation and expansion processes can vary significantly between institutions, and there are only a few techniques (e.g., Dynabeads®) available for commercial development. The effector function, persistence and engraftment are also clinically significant characteristics of the expanded T cells. The relative importance assigned to these properties also varies between institutions. Despite the variation in approaches to cell manufacturing, many T cell therapies are in clinical trials. The disclosed subject matter provides aspects of alternative activation and expansion reagents for the manufacture of adoptive cell therapies ex vivo.

T-cell activation and expansion ex vivo can be initiated after agonistic anti-CD3 antibodies bind to TCR complexes. The activation is enhanced if additional co-stimulatory signals are provided to the T-cells, and anti-CD28 antibody molecules are used frequently to provide this signal ex vivo. Therefore, receptor cross-linking of T cell surface receptors (e.g., TCR and CD28) is significant for the activation and expansion of T cells. Researchers have been developing reagents to recapitulate ex vivo the endogenous agonistic and co-stimulatory signals of T cell activation, expansion and proliferation. CD3 is a multimeric protein that acts as a co-receptor with the TCR. Binding and co-localization by monoclonal anti-CD3 antibodies caused T cell activation and proliferation in early studies (Turka 1990, Riddell 1990). Since early studies were performed with mixtures of cells (T cells and other blood cell types), the role of accessory, or feeder, cells (e.g., APCs) in T cell activation was unknown. Further complicating the issue were conflicting results with other T cell stimulating factors (e.g., plant lectins, interleukin-1, interleukin-2 (IL-2) and tetradecanoyl phorbol acetate) used singly or in combination with anti-CD3 Ab (Rosenberg 1988, Riddell 1990, Dixon 1989). In vivo, B7 proteins on APCs engage the CD28 co-receptor on T cell surfaces as a co-stimulatory signal, so anti-CD28 Abs will elicit similar responses. In fact, co-stimulatory anti-CD28 mAbs were shown to augment T cell expansion in the presence of accessory cells (Riddell 1990). Other co-stimulatory receptors on T cells include 4-1BB and CD27 (Eggermont 2014). By the early 1990s, there were two major questions regarding T cell activation ex vivo: the role of accessory cells (e.g., APCs) and the role of receptor cross-linking (e.g., TCR and co-stimulatory receptors). It was found that soluble anti-CD3 did not induce T cell proliferation in the absence of accessory cells (Dixon 1989, Levine 1997). However, when anti-CD3 was immobilized to a surface (e.g., microwell), its presence did cause T cell proliferation (Dixon 1989). The soluble vs. plate-bound result was confirmed with anti-CD3 and anti-CD28 in combination, as well (Levine 1996, Lamers 1992). It was hypothesized that plate-bound Abs permitted TCR cross-linking with co-stimulatory receptors for T cell proliferation ex vivo, and presaged the development of bead-bound anti-CD3/anti-CD28 reagents for T cell expansion.

The higher surface area to volume ratio of microbeads (magnetic or otherwise) made them attractive platforms to present signals to T cells for two reasons. First, higher antibody binding capacity on microbead surfaces (compared to microwell surfaces) allowed more pro-expansion signaling molecules to engage T cell receptors. Second, a multiplicity of anti-CD3 and anti-CD28 antibodies on bead surfaces enabled efficient TCR cross-linking for T cell activation and expansion, and it better recapitulates the APC-T cell interaction (Trickett 2003). The anti-CD3/anti-CD28 bound microbeads can be thought of as artificial antigen presenting cells (aAPCs) without antigen or HLA. The Dynabeads CD3/CD28 CTS™ magnetic microbeads are cell-sized (˜2 μm) and used in clinical research for CART cell therapies (Kaiser 2015). T cell activation and expansion is important for adoptive T cell therapy not only to produce sufficient effector cells to elicit a pharmacologic response in the patient but also to increase the transduction efficiency of the CAR construct. For example, anti-CD3/anti-CD28 conjugated magnetic microparticles (Dynabeads) induced expansion of non-specific CD4+ T cells over several weeks, and proliferation was improved with additions of the cytokine interleukin-2 (IL-2) (Levine 1997, Levine 1996). IL-2 is a common supplement that provides a co-stimulatory signal during T cell expansion and often produces more robust expansion of CD8+ T cells (Lamers 1992). Bead-bound anti-CD3 and anti-CD28 also caused activation and expansion of tumor-reactive cytotoxic CD8+ T cells (Tescher 2011, Maus 2002), however additional co-stimulatory signals showed better results than anti-CD3 and anti-CD28 alone. These co-stimulatory domains have been engineered into CAR constructs in the later generation of CAR T cell therapies (Maus 2002, Jensen 2015). Therefore, the anti-CD3/anti-CD28 bound microbeads can be used to activate and expand both major T cell subsets (CD4+ and CD8+) into effector T cells. However, the utility of T cell activation is not limited to CAR T cell therapies. There are other research and therapeutic applications that require T cell activation.

Miltenyi Biotec, a competitor of Dynabeads, markets the MACS GMP TransAct CD3/CD28 reagent kits for research use only. This type of aAPC includes a nanomatrix with magnetic nanoparticles embedded in a polymeric matrix that has surface-conjugated anti-CD3 and anti-CD28 mAbs. Its particle size is ˜100 nm. The subject embodiments provide methods and reagents to activate and expand T cells ex vivo without the need for extraneous reagents like magnetic microbeads or nanoparticles.

There are other aAPC technologies for cell activation still in research phases. There are two broad application areas for aAPCS: in vivo or ex vivo. The goal of in vivo aAPC treatments is to present antigen to T cells in order to elicit an antigen specific response in effector cells. The goal of ex vivo APCs or aAPCs is to activate and expand (typically naïve) T cells (either non-specific or antigen specific) in sufficient numbers for infusion into patients. The described technology is designed as a novel type of aAPC with additional functionality (e.g., receptor clustering and ligand concentration) for use ex vivo. The ex vivo expansion of T cells can be controlled to generate effector T cell populations with different properties (e.g., central memory, cytotoxic T-lymphocyte). Cell-based aAPCs include cell lines that are genetically engineered to induce T cell responses. These cell-based aAPCs can be engineered to express Fc receptors, T cell activation molecules and T cell co-stimulatory molecules (Butler 2013). These cell-based aAPCs can expand T cells for tumor infiltrating lymphocyte (TIL) therapy and other therapeutic areas (Forget 2014).

Synthetic aAPCs are completely acellular, but they can be engineered to present stimulatory (e.g., CD3), co-stimulatory (e.g., CD28, 4-1BB) and even ‘self’ (Bruns 2015) signals to T cells. The most widely used example of synthetic aAPCs is Dynabeads, which are polymer-coated magnetic microparticles with surface conjugated stimulatory (anti-CD3 Ab) and co-stimulatory (anti-CD28 Ab) molecules (Porter 2006), as described above. Another example of an aAPCs is a biodegradable particle system that releases cytokines over time for CD8+ T cell specific proliferation (Steenblock 2011). Other aAPCs are designed with non-spherical shapes that better mimic the complex morphology of APCs (Sunshine 2014). These synthetic aAPCs may be easily customized in terms of molecule presentation, molecule density and shape, and they permit mechanistic studies of T cell activation.

Although microns-sized particles as aAPCs may induce better T cell responses than nanoparticles (Steenblock 2011), nano-sized aAPCs have also been developed for T cell activation. These nano-aAPCs are advantageous for in vivo antigen presentation because they are small enough for systemic circulation without the risk of embolisms inherent with larger microparticles. Nano-aAPCs also have advantages for non-specific or antigen specific T cell activation and expansion ex vivo. The Miltenyi Biotec MACS GMP TransAct CD3/CD28 system is one example of a nano-aAPC. Other nanoparticles were used to generate and expand antigen-specific CD8+ mouse T cells (Perica 2015) but the use of nano-aAPCs for human applications is still in its infancy. The current disclosure describes a stimuli-responsive polymer-based technology for cell activation and expansion that is an improvement on nano- and micro-sized APCs.

Furthermore, the production of the disclosed polymer-Ab conjugates is considerably simpler, and as such, cheaper and more time-efficient, than the production of ‘nanoworms’, such as nanoworms with multiple Ab conjugation sites. Also, as noted herein, co-aggregation caused by the application of a stimulus such as a thermal or chemical stimulus is completely reversible, such as by dis-aggregation. Such a level of control is not possible with other aAPC systems and as such, the subject methods and compositions are more efficient than those of such aAPC systems.

### VI. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### A. Example 1

### Polymer Synthesis, Modification and Characterization

Three temperature-responsive polymers or copolymers were synthesized via reversible addition-fragmentation chain transfer (RAFT) polymerization. The polymer poly(NIPAM) comprised the temperature responsive monomer N-isopropylacrylamide (NIPAM). The copolymers, poly(NIPAM₉-co-BA₁) or poly(NIPAM₃₅-co-BA₁), comprised both temperature-responsive NIPAM and hydrophobic (butyl acrylate, or BA) monomers. NIPAM, BA (if used), chain transfer agent (CTA) 4-Cyano-4-(ethylsulfanylthiocarbonyl)sulfanyl pentanoic acid, free radical initiator 4-4′-azobis(cyanovaleric acid) (ACVA) and dimethylformamide (DMF) were sealed in a round bottom flask. The molar ratio of NIPAM to BA was 9:1 or 35:1 for the copolymers, and the target degree of polymerization (DP) was 400:1. The flask was purged with N₂ for 20 minutes and then heated to 70° C. for 4 hours.

Following the reaction, all of the polymers were isolated by 4 repeated rounds of precipitation into a 4/1 (v/v) mixture of pentane/diethyl ether following acetone dissolution and dried overnight in vacuo. Subsequently, the polymers' terminal carboxylic acid groups were converted to an amine-reactive NHS ester (FIG. 10) with N-hydroxysuccinirnide (NHS) and N,N′-diisopropylcarbodiimide (DIC). The reaction proceeded overnight at room temperature in chloroform at a polymer concentration of 70 mg/mL, a 5:1 (mol:mol) ratio of NHS to polymer, and a 2:1 (mol:mol) ratio of DIC to NHS. The activated polymer, poly(NIPAM)-NHS, and copolymers, poly(NIPAM₉-co-BA₁)-NHS or poly(NIPAM₃₅-co-BA₁)-NHS, were isolated by 3 repeated rounds of precipitation into diethyl ether following chloroform dissolution and dried overnight in vacuo. All polymers were analyzed by size exclusion chromatography (SEC) to measure the molecular weight and size distribution (PDI). The chemical compositions and purities of the polymers were confirmed by ¹H-NMR at 500 MHz. The efficiency of NHS activation was confirmed by UV/Vis spectroscopy.

### B. Example 2

### Polymer-Antibody Conjugate Preparation and Characterization

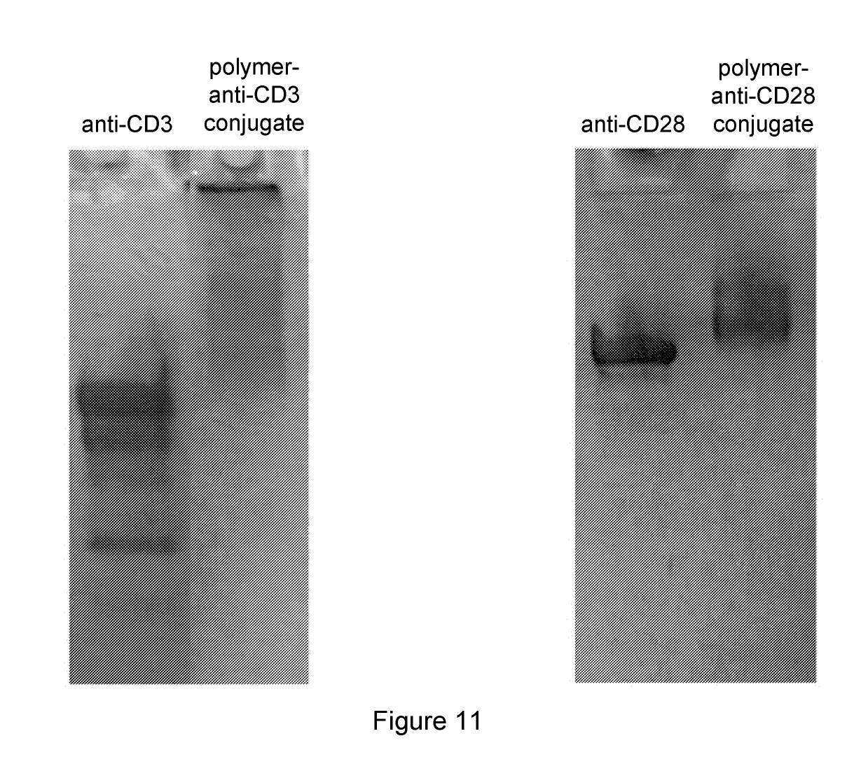

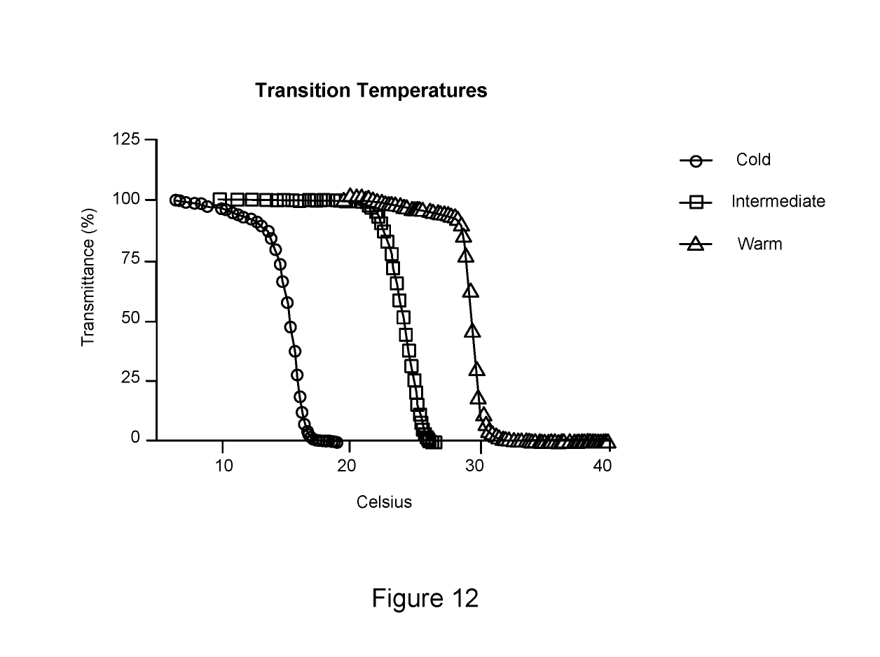

Monoclonal anti-CD3 (clone OKT-3) or monoclonal anti-CD28 (clone 9.3) were covalently linked to poly(NIPAM)-NHS, poly(NIPAM₉-co-BA₁)-NHS and poly(NIPAM₃₅-co-BA₁)-NHS as follows. The Ab was diluted into sodium bicarbonate buffer (pH 9.5) and cooled. The Ab solution was added to the polymer or copolymer solutions and mixed for 18 hours at 4° C. Covalent conjugation occurred at accessible amine groups on Ab molecules. The polymer:Ab molar ratio for conjugations was 10:1 or 20:1. Free Ab was removed by thermally aggregating the polymer-Ab conjugates, centrifuging the aggregates and removing the supernate containing unconjugated Ab. As is illustrated, for example in FIG. 11, polymer conjugation to the Ab molecules was confirmed by SDS-PAGE analysis. The degree of conjugation was estimated via molecular weight calculations from aqueous SEC traces of the polymer-Ab conjugates. The temperature response, or lower critical solution temperature (LCST), of the polymer-Ab conjugates was measured by cloud point measurements in a temperature-controlled UV-Vis spectrophotometer. Anti-CD3 conjugated to poly(NIPAM), poly(NIPAM₉-co-BA₁) or poly(NIPAM₃₅-co-BA₁) exhibited temperature-induced transition at approximately 32° C., 18° C. and 26° C., respectively, as illustrated in FIG. 12. The graph illustrates polymer-antibody conjugates responding to different temperature stimuli.

### C. Example 3

### Binding Function of Polymer-Ab Conjugates

Binding curves of the polymer-Ab conjugates and unconjugated Abs were generated in order to confirm that polymer conjugation did not alter the binding specificity of the polymer-Ab conjugates. Polymer-anti-CD3 conjugates or unconjugated anti-CD3 were added to Jurkat cells, a T lymphocyte cell line, at concentrations between 1-10 μg/mL. After binding for 25 minutes on ice, the cells were washed once to remove unbound Ab species. Then, goat anti-human Fc Alexa Fluor 647 Ab was added to the cells for 20 minutes on ice to bind the polymer-Ab conjugates or Ab on the cell surfaces. After washing the cells to remove unbound secondary Ab, the cells were analyzed by flow cytometry. This binding curve experiment was repeated with unconjugated anti-CD28 and polymer-anti-CD28 conjugates. Minimal differences in the binding behaviors of the unconjugated or conjugated Abs were noted.

### D. Example 4

### Magnetic Nanoparticle (mNP) Synthesis and Purification

In this example, the preparation, characterization, and use of representative temperature-responsive nanoparticles of the disclosure are described. When a stimuli-responsive polymer that does not include a micelle-forming group at any polymer terminus was used in the process of the present disclosure, a mNP with some unique properties was generated when compared with a mNP that was made using a stimuli-responsive polymer that includes a micelle-forming group at a polymer terminus. However, mNPs can be produced with polymers containing a micelle-forming group or without a micelle-forming group. Accordingly, this example demonstrates the ease of synthesis and reproducibility of embodiments of stimuli-responsive mNPs of the present disclosure.

i. Synthesis of Precursor Reagents

Iron oleate (Fe-oleate₃) was used as the iron source for stimuli-responsive magnetic nanoparticles (mNPs). The Fe-oleate₃ was prepared according to previously published methods. See, e.g., Park et al., Nature Materials, 3:891-895, 2004.

Briefly, sodium oleate (TCI, >97%) and iron (III) chloride hexahydrate (Sigma Aldrich, 97%) were dissolved in a mixed solvent system of hexanes, ethanol and deionized water. The solution was refluxed for 4 hours at 70° C., washed thrice in deionized water and dried to yield a viscous, dark red oil. Yield=75%. Composition was confirmed by ¹H-NMR on a Bruker Avance spectrometer operating at 300 MHz in CDCl₃.

In this example, the stimuli-responsive polymer responded to changes in temperature. The temperature-responsive, hydrophilic, random copolymer comprising NIPAM and BA monomers was synthesized using reversible addition-fragmentation chain transfer (RAFT) techniques, as described, for example, in Chiefari, J. et al., Macromolecules 31: 5559-5562, 1998. In detail, copolymers of NIPAM and BA were synthesized via RAFT polymerization. The reaction was conducted in DMF at 70° C. under a nitrogen atmosphere for 2 h using the CTA 2-(dodecylthiocarbonothiolylthio)-2-methylpropionic acid and free radical initiator ACVA. The molar ratio of NIPAM to BA was 9:1, and the target DP was 50:1. Following the reaction, the poly(NIPAM-co-BA) was isolated following 4 repeated rounds of precipitation into a 4/1 (v/v) mixture of pentane/diethyl ether following acetone dissolution and dried overnight in vacuo.

The molecular weight and molecular weight distribution were determined via size exclusion chromatography with multi-angle laser light scattering (Wyatt miniDAWN TREOS) and refractive index (Wyatt Optilab T-rEX) detectors. The polymer composition was confirmed by ¹H-NMR spectroscopy, as described above.

Next, the copolymer was subjected to radical induced end group reduction to remove the CTA end group. This was performed by reacting the copolymer with 1-ethylpiperidine hypophosphite (EPHP) and 1,1′-azobis(cyclohexanecarbonitrile) (ACHN) under a nitrogen atmosphere in DMF at 95° C. for 4 h. The molar ratio of EPHP to copolymer was 15:1, the molar ratio of ACHN to copolymer was 1:1, and the concentration of copolymer was 150 mg/mL. Following the reaction, the ‘cleaved’ copolymer was isolated by precipitation into a 3/1 (v/v) mixture of pentane/diethyl ether and dried overnight in vacuo. The resultant material was then dialyzed against dH₂O with a 3.5 kDa MWCO membrane at 4° C. and lyophilized. The cleaved copolymer was characterized by ¹H-NMR and SEC, as above.

ii Synthesis and Purification of mNPs

Magnetic nanoparticles (mNPs) were produced via thermal decomposition of iron oleate in the presence of cleaved copolymer as described in WO 2014/194102 (PCT/US2014/040038). The reaction was carried out at 190° C. for 6 h in tetraethylene glycol dimethyl ether at a 10:1 molar ratio of iron oleate to cleaved copolymer and a cleaved copolymer concentration of 18 mg/mL. Following the reaction, mNPs were isolated by three repeated rounds of precipitation into pentane with acetone dissolution and dried overnight in vacuo. The dried mNPs were then dissolved in dH₂O and further purified by tangential flow filtration (TFF) against dH₂O with a 100K MWCO polyethersulfone membrane filter, passed through a 0.45 μm polyvinylidene fluoride syringe filter, and then lyophilized.

iii. Characterization of mNPs

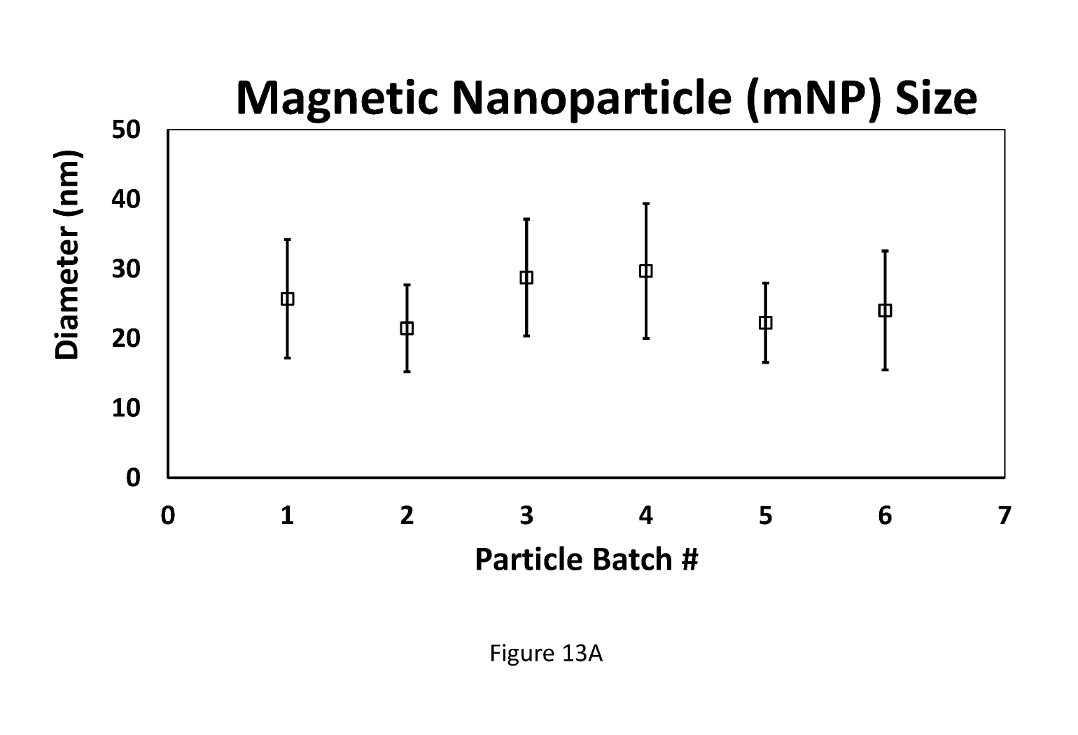

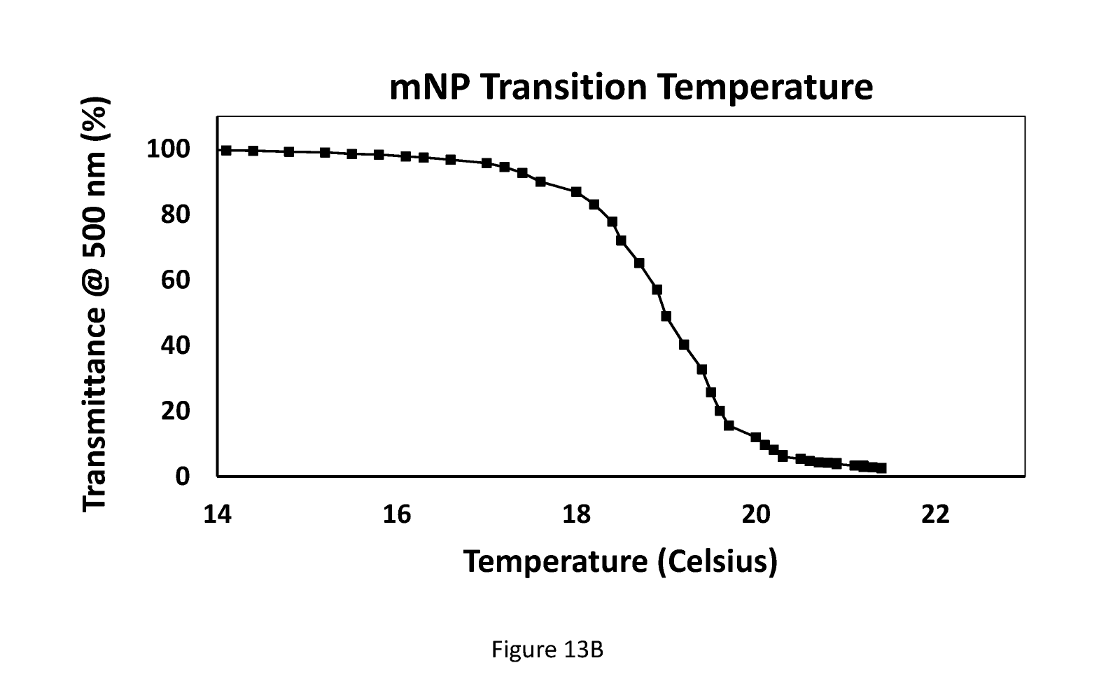

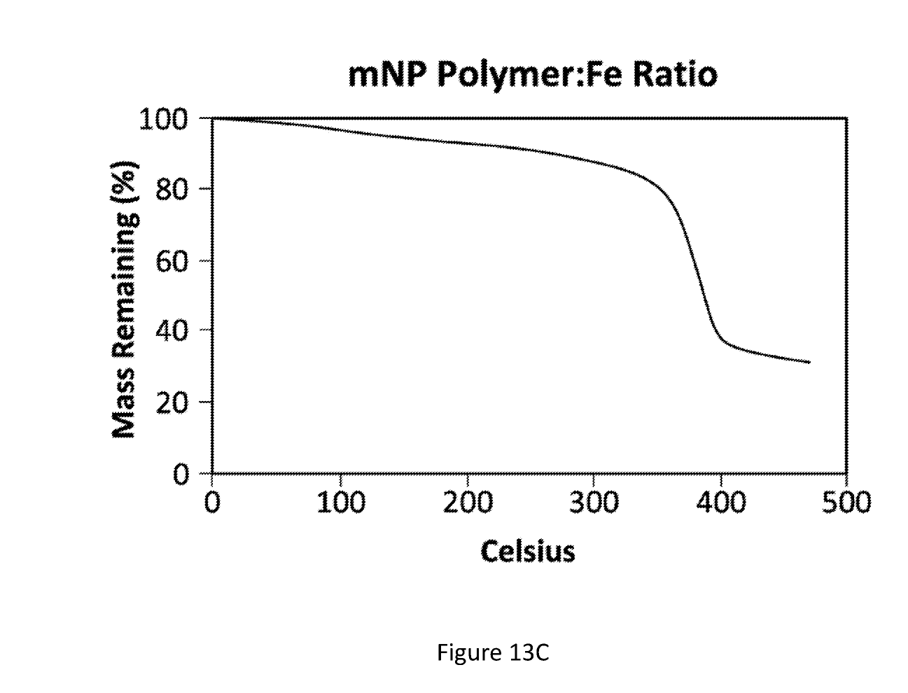

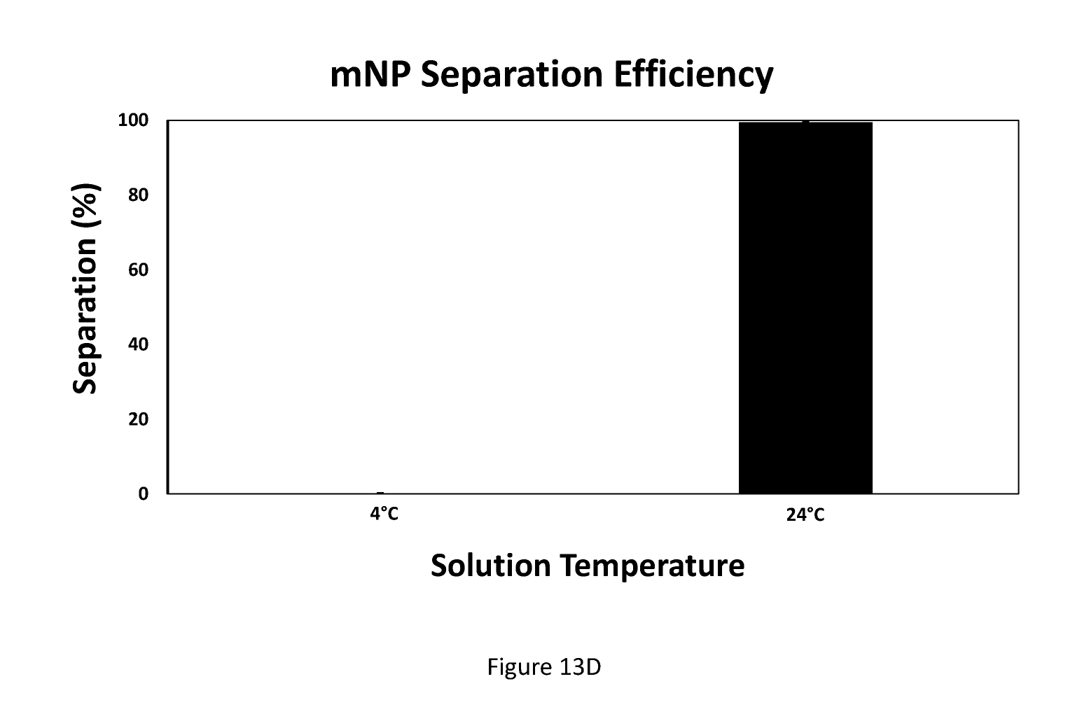

The hydrodynamic size of the mNPs was measured by dynamic light scattering (DLS) with a Malvern Zetasizer Nano-ZS instrument an mNP concentration of 1 mg/mL in 10 mM phosphate buffered saline. The number-weighted average diameter for six different mNP batches is presented in FIG. 13A and Table 1.

The lower critical solution temperature (LCST) was determined by measuring the visible light transmittance of a mNP solution with a UV-Visible spectrophotometer as the mNPs were heated from 4° C. to 25° C. (FIG. 13B). At the LCST (16±1° C.), the mNPs aggregated, resulting in a decrease in solution transmittance.

The polymer: iron mass ratio was measured by thermogravimetric analysis (TGA) on a TA Instruments TGA Q50 (FIG. 13C).

The mNP separation efficiency is a measure of how many mNPs can be separated from a solution after 2 minutes of exposure to a simple magnet. A mNP solution (1 mg/mL in HBSS with 5% serum) was exposed to 4° C. or 24° C. conditions for 2 minutes. The solution was kept at 4° C. or 24° C. and then placed in a magnetic holder for 2 minutes. The absorbance (500 nm) of the supernatant was measured on a VWR UV-3100P spectrophotometer. The loss of absorbance was compared to a mNP control solution and quantified as the separation efficiency (FIG. 13D).

TABLE 1
Structural and functional properties of stimuli-responsive
mNPs (data presented as the mean ± standard deviation
for six different mNP batches, and small deviations indicate
reproducibility of mNP production process).

| Property | Measurement |
|---|---|
| Size | 25 nm ± 3.0 nm |
| 4° C. separation | 1.0% ± 2.0% |
| 24° C. separation | 99% ± 1.0% |
| LCST | 16° C. ± 1.0° C. |
| Polymer:Fe mass ratio | 2.0 ± 0.20% |

iv. Results

The Fe-oleate₃ complex composition was determined by ¹H-NMR, which showed appropriate chemical shifts and integration values for oleic acid. The hydrophilic, stimuli-responsive polymer was 6.0±0.20 kDa (n=3), as measured by size exclusion chromatography.

FIG. 13A shows the hydrodynamic diameter (number-weighted averages) of six different batches of mNPs, as measured by DLS. The diameters of these mNP batches ranged from 22 nm to 30 nm. The standard deviation of these measurements ranged from 6 to 10 nm. The LCST describes the temperature at which hydrophilic polymers aggregate into hydrophobic agglomerates, and is measured by the cloud point, or solution transmittance (FIG. 13B). The LCST of mNPs synthesized with NIPAM-BA copolymers was typically around 15-20° C. Therefore, incorporation of the polymer with the mNPs did not affect significantly the stimuli-responsive behavior of the polymer.

During TGA, dry samples are heated rapidly, which cause organic material to vaporize or combust, thus leading to a decrease in sample mass. Here, TGA was used to measure the amount of polymer incorporated around the inorganic iron oxide nanoparticle core of the mNPs. The mass loss at ˜100° C. was about 5% and represented the loss of residual water that was not removed by lyophilization. The polymer decomposition occurred over a broad range of temperatures (˜250-400° C.). The remaining mass was typically 32%, which represented the iron oxide core of the mNPs. The polymer:Fe mass ratio is shown in Table 1, and was calculated from the major decomposition mass loss and the remaining mass.

The separation efficiency (FIG. 13D) is an important functional property of the mNPs. Below the LCST at ˜16° C., the mNPs were soluble and too small for separation with a simple magnet (separation efficiency <5%). Above the LCST, the mNPs formed large aggregates that were easily separated with a simple magnet (separation efficiency ˜99%). Therefore, the stimuli-responsive behavior of the mNPs translates to a useful functional characteristic.

FIGS. 13B-13D show data from a single representative mNP batch. The same properties (average±standard deviation) are shown in Table 1 from six different batches of mNPs. The small standard deviations show that the mNP production yields mNPs with reproducible structural and functional properties.

### E. Example 5

### Activation of Cells Using Two Different Polymer-Affinity Reagent Conjugates to Cluster Different Cell Surface Receptors

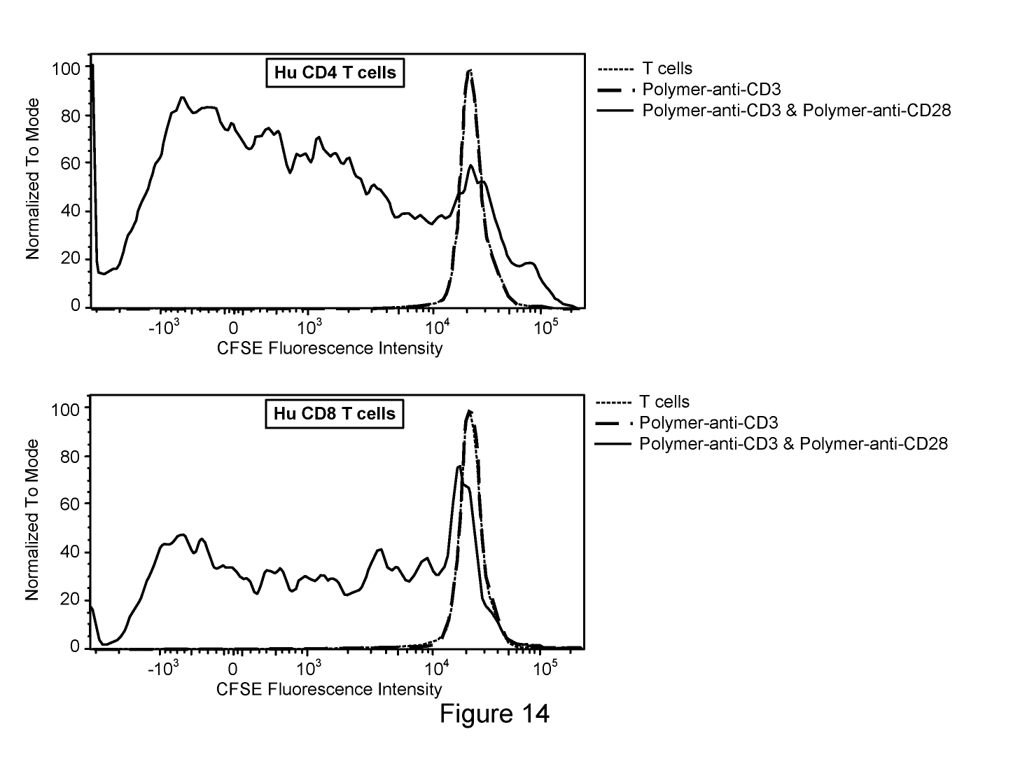

Polymer-anti-CD3 conjugates alone or in addition to polymer-anti-CD28 conjugates were added to purified human T cells on ice, which allowed the conjugates to bind to the CD3 (part of the T cell receptor, or TCR) and/or CD28 molecules on the T cell surfaces. The cells were warmed to 37° C. and placed in an incubator. The increase in temperature from ˜4° C. to 37° C. served as the stimulus for polymer-Ab conjugates to co-aggregate. Thus, the polymer-anti-CD3 and polymer-anti-CD28 conjugates co-aggregated at the cell surfaces and caused CD3/CD28 receptor clustering. The polymer-anti-CD3 conjugates also co-aggregated but caused only CD3 receptor clustering and co-localization. T cell proliferation was measured by CFSE dilution using flow cytometry analyses after 5 days of culture in the presence of the conjugates. In the presence of polymer-conjugated anti-CD3 alone, the T cells were not activated, as shown by the single sharp fluorescence peak, presumably because more than one signal is required for optimal T cell activation. In the presence of polymer-anti-CD3 and polymer-anti-CD28 according to the subject disclosure, both CD4+ (top panel of FIG. 14) and CD8+ (bottom panel of FIG. 14) T cells proliferated and underwent multiple population doublings, as shown by the multiple fluorescent peaks. Therefore, polymer-Ab conjugates are able to cross-link TCRs and engage co-stimulatory CD28 molecules on T cell surfaces, leading to T cell activation ex vivo.

The following provides a more specific method of using two different polymer-affinity reagent conjugates to activate T cells ex vivo. The anti-CD3 Ab (clone OKT3) binds to the CD3 molecule associated with T cell receptors (TCRs). Engagement and co-localization of CD3 with surface-bound agonistic anti-CD3 antibodies like clone OKT3 activates T cells. CD28 is a molecule on T cell surfaces that must be engaged by endogenous (B7 proteins) or other (anti-CD28 Ab, clone 9.3) molecules in conjunction with TCR engagement for maximal T cell activation.

Temperature responsive polymers were conjugated to anti-CD3 Abs and anti-CD28 Abs and analyzed by SDS-PAGE. Results of the process are provided in FIG. 11. The gels showed that the polymer-Ab conjugates were larger than free Ab and likely contained Ab molecules with different numbers of polymers conjugated to them. These polymer-anti-CD3 and polymer-anti-CD28 conjugates were added to purified human T cells on ice, which allowed the conjugates to bind to the CD3 and CD28 molecules on the T cell surfaces. The cells were warmed to 37° C. and placed in an incubator. The increase in temperature from ˜4° C. to 37° C. served as the stimulus for Ab-polymer co-conjugate aggregation. Thus, the polymer-anti-CD3 and polymer-anti-CD28 conjugates co-aggregated at the cell surfaces and caused CD3/CD28 receptor cross-linking. T cell proliferation was measured by CFSE dilution at day 5 using flow cytometry analyses as provided in FIG. 14. In the presence of polymer-conjugated anti-CD3 alone, the T cells were not activated, as shown by the single sharp fluorescent peak, presumably because more than one signal is required for optimal T cell activation. However, in the presence of polymer-anti-CD3 and polymer-anti-CD28, both CD4+ and CD8+ T cells proliferated and underwent multiple population doublings, as is shown by the multiple fluorescent peaks. Therefore, polymer-Ab conjugates were able to cross-link TCRs and induce T cell activation and expansion ex vivo.

### F. Example 6

### Activation of Cells Using Two of the Same Polymer-Affinity Reagent Conjugates to Cluster the Same Cell Surface Receptors

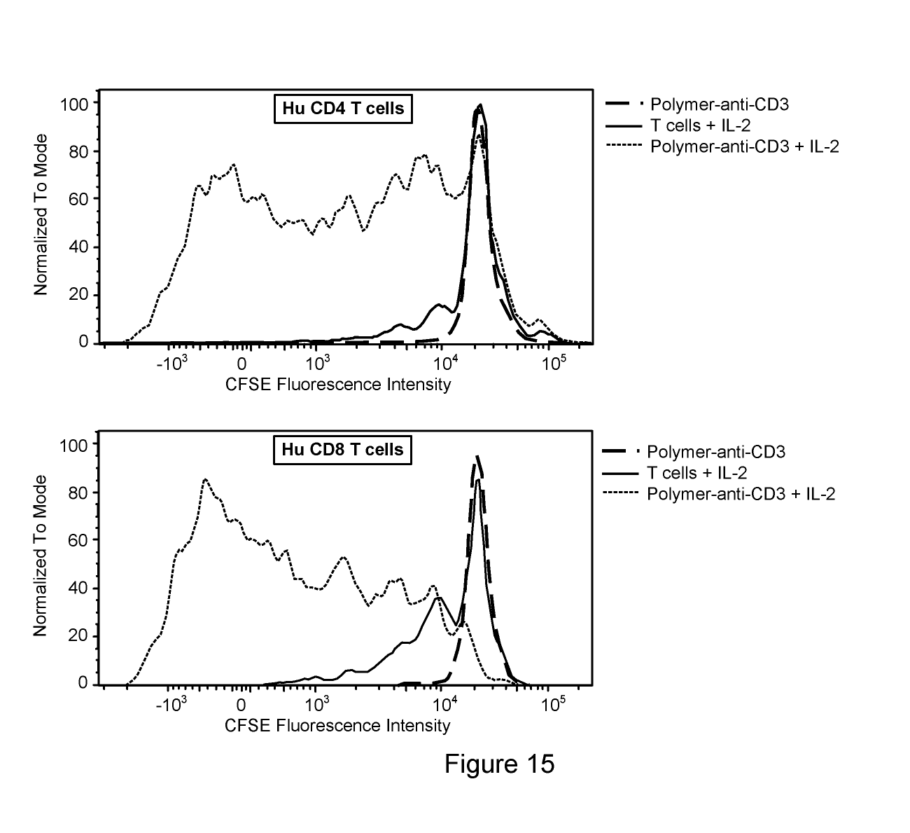

Polymer-anti-CD3 conjugates were added to purified human T cells on ice, which allowed the conjugates to bind to the CD3 (part of the T cell receptor, or TCR) molecules on the T cell surfaces. The culture media was supplemented with 200 IU/mL interleukin-2 (IL-2), a cytokine that functions as a soluble co-stimulatory signal for T cell activation. There were two control conditions. T cells were treated with 200 IU/mL IL-2 but not the polymer-anti-CD3 conjugates or T cells were treated with the polymer-anti-CD3 conjugate but no IL-2. The cells were warmed to 37° C. and placed in an incubator. The increase in temperature from ˜4° C. to 37° C. served as the stimulus for polymer-Ab conjugates to co-aggregate. Thus, the polymer-anti-CD3 conjugates co-aggregated at the cell surfaces and caused CD3 receptor clustering and cross-linking. T cell proliferation was measured by CFSE dilution using flow cytometry after 5 days of culture. T cells with IL-2 proliferated slightly because IL-2 provides a co-stimulatory signal to the cells, as shown by the shoulders to the left of the dominant fluorescence peak. T cells treated with polymer-anti-CD3 conjugate did not proliferate, as shown by the single fluorescence peak. In the presence of polymer-anti-CD3 and IL-2 both CD4+ (top panel of FIG. 15) and CD8+ (bottom panel of FIG. 15) T cells were highly activated; they proliferated and underwent multiple population doublings, as shown by the multiple fluorescent peaks. Therefore, polymer-Ab conjugates are able to cross-link TCRs on T cell surfaces, leading to T cell activation ex vivo in the presence of an additional soluble co-stimulatory signal.

The following provides a more specific method of using two of the same polymer-affinity reagent conjugates to activate T cells ex vivo. The anti-CD3 Ab (clone OKT3) binds to the CD3 molecule associated with T cell receptors (TCRs). Agonistic anti-CD3 antibodies like clone OKT3 can engage the TCR but the antibody typically needs to be surface bound (e.g., attached to microparticles) for TCR clustering and subsequent T cell activation. Even when TCRs are clustered, T cell activation can be muted without the presence of an additional, co-stimulatory signal. IL-2 and CD28 are examples of co-stimulatory molecules. IL-2 is a soluble cytokine involved in T cell differentiation, and it provides a co-stimulatory signal for T cell activation. CD28 is molecule on T cell surfaces that must be engaged by endogenous (B7 proteins) or other (anti-CD28 Ab, clone 9.3) molecules in conjunction with TCR engagement for maximal T cell activation. It can also be cross-linked with TCR.

Temperature responsive polymers were conjugated to anti-CD3 Abs and analyzed by SDS-PAGE. Results of the process are provided in FIG. 11. The gels showed that the polymer-Ab conjugates were larger than free Ab and likely contained Ab molecules with different numbers of polymers conjugated to them. These polymer-anti-CD3 conjugates were added to purified human T cells on ice, with or without IL-2. At the lower temperature, the conjugates bind to the CD3 molecules and the IL-2 binds to IL-2 receptors on the T cell surfaces. The cells were warmed to 37° C. and placed in an incubator. The increase in temperature from ˜4° C. to 37° C. served as the stimulus for Ab-polymer conjugate co-aggregation. Thus, the polymer-anti-CD3 conjugates co-aggregated at the cell surfaces and caused CD3 receptor cross-linking. T cell proliferation was measured by CFSE dilution at day 5 using flow cytometry as provided in FIG. 15. In the presence the co-stimulatory signaling molecule IL-2, the T cells proliferated slightly. With polymer-anti-CD3 but not IL-2, the T cells were not activated, as shown by the single sharp fluorescence peak. However, in the presence of polymer-anti-CD3 and IL-2, both CD4+ and CD8+ T cells proliferated and underwent multiple population doublings, as is shown by the multiple fluorescent peaks. Therefore, polymer-Ab conjugates were able to cross-link TCRs and induce T cell activation and expansion ex vivo, in the presence of an additional co-stimulatory signal.

### G. Example 7

### Activation of Cells Using Three of the Same Polymer-Affinity Reagent Conjugates, with Different Temperature-Responsive Behavior, to Cluster the Same Cell Surface Receptors

The following provides a specific method of using two of the same polymer-affinity reagent conjugates to activate T cells ex vivo. Examples are given for several Ab-polymer conjugates, each with differing aggregation thresholds. The anti-CD3 Ab (clone OKT3) binds to the CD3 molecule associated with T cell receptors (TCRs). Agonistic anti-CD3 antibodies like clone OKT3 can engage the TCR but the antibody typically needs to be immobilized (i.e., attached to microparticles or culture plate surfaces) in order to enable TCR clustering and subsequent T cell activation. Even when TCRs are clustered, T cell activation can be muted without the presence of an additional, co-stimulatory signal. IL-2 and CD28 are examples of co-stimulatory molecules. IL-2 is a soluble cytokine involved in T cell differentiation, and it provides a co-stimulatory signal for T cell activation. CD28 is molecule on T cell surfaces that must be engaged by endogenous (B7 proteins) or other (anti-CD28 Ab, clone 9.3) molecules in conjunction with TCR engagement for maximal T cell activation. It can also be cross-linked with TCR.

Temperature responsive polymers were conjugated to anti-CD3 Abs and analyzed by SDS-PAGE. Results of the process are provided in FIG. 11. The gels showed that the polymer-Ab conjugates were larger than free Ab and likely contained Ab molecules with different numbers of polymers conjugated to them.

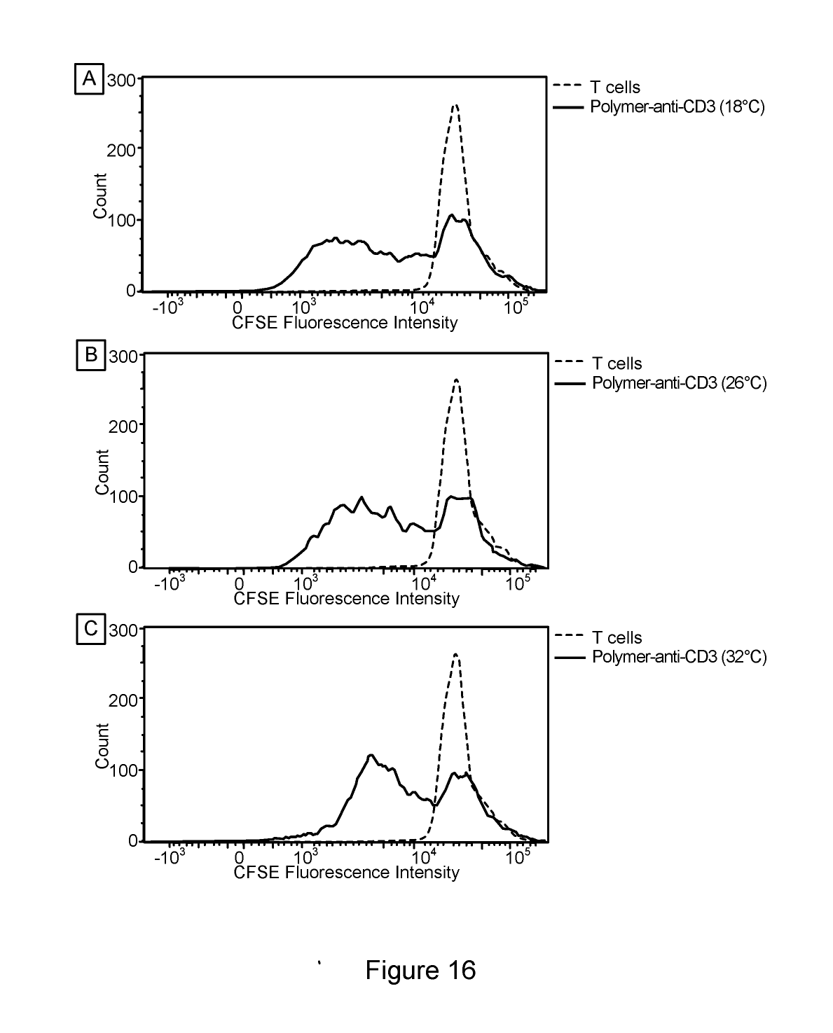

Three polymer-anti-CD3 conjugates that respond to either 18° C., 26° C. or 32° C. temperature stimuli (Example 2 and FIG. 12) were added to purified human T cells on ice, which allowed the conjugates to bind to the CD3 (part of the T cell receptor, or TCR) molecules on the T cell surfaces. The culture media was supplemented with 200 IU/mL interleukin-2 (IL-2), a cytokine that functions as a soluble co-stimulatory signal for T cell activation. There was one control condition: T cells were treated with 200 IU/mL IL-2 but not the polymer-anti-CD3 conjugates. The cells were warmed to 37° C. and placed in a 37° C. incubator with a 5% CO₂ atmosphere. The increase in temperature from ˜4° C. to 37° C. served as the stimulus for polymer-Ab conjugates to co-aggregate. Thus, the polymer-anti-CD3 conjugates co-aggregated at the cell surfaces and caused CD3 receptor clustering and cross-linking. T cell proliferation was measured by CFSE dilution using flow cytometry after 4 days of culture, for example in FIG. 16. T cell cultures without an activation signal did not proliferate, as shown by the single fluorescence peak (dotted lines). T cell cultures treated with polymer-anti-CD3 conjugates that respond to temperature stimuli of 18° C. (FIG. 16A), 26° C. (FIG. 16B) and 32° C. (FIG. 16C) were highly activated; they proliferated and underwent multiple population doublings, as shown by the multiple fluorescent peaks. Therefore, polymer-anti CD3 Ab conjugates that respond to different temperature stimuli are able to cross-link TCRs on T cell surfaces, leading to T cell activation ex vivo.

### I. Example 8

### Polymers and Magnetic Nanoparticles that Respond to Both Temperature and Ionic Strength

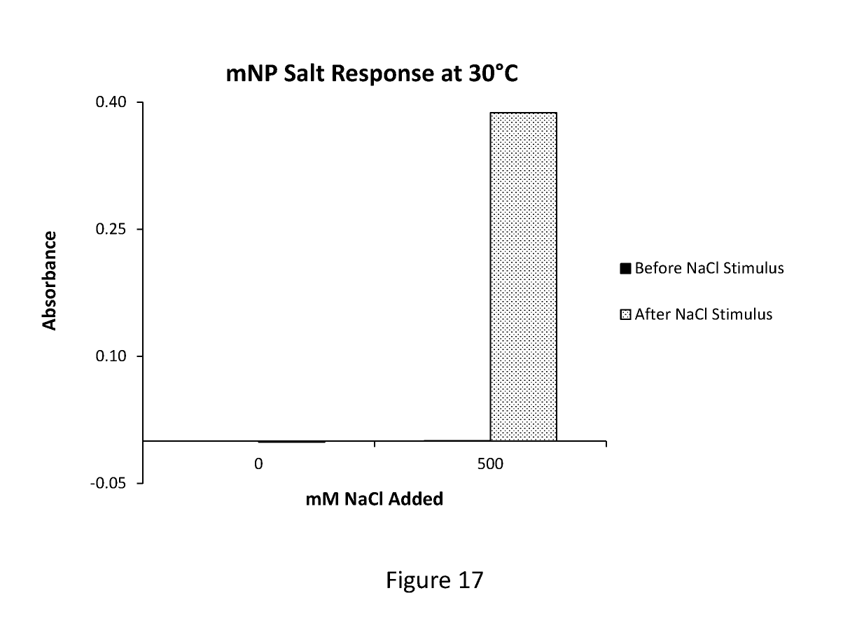

The temperature-responsive polymer, poly(NIPAM), was synthesized, purified and characterized as described in Example 4. This polymer was then used to produce magnetic nanoparticles (mNPs) as described in Example 4. The temperature- and ionic-strength responsive behavior was monitored by cloud point assays. Solutions of mNPs (2 mg/mL) were made in 10 mM phosphate buffer (PB), pH 7.4. Then, the mNPs were brought to 30° C., and the absorbance (500 nm wavelength) was measured. The mNPs were not aggregated because their LCST was ˜32° C. A stock solution of 1 M NaCl was used to add either 0 mM or 500 mM additional NaCl to the mNPs. As is shown, for example in FIG. 17, after bringing the mNPs to 30° C. again and measuring the absorbance (500 nm), the higher absorbance was due to mNP aggregation in response to the 500 mM NaCl ionic strength stimulus. Thus, polymers, magnetic nanoparticles and polymer-modified entities such as Abs can be made to respond to more than one stimulus.

### J. Example 9

### Isolation of Monoclonal Antibodies (mAb) from Solutions with Polymer—Protein A Conjugates

Stimuli-responsive polymer, poly(NIPAM₉-co-BA₁), was synthesized, purified, activated and characterized as described in Example 1. Protein A was covalently linked to the poly(NIPAM₉-co-BA₁)-NHS as described in Example 2. The polymer:protein A molar offering ratio for conjugations was 25:1. Unconjugated protein A was removed by thermally aggregating the polymer-Protein A conjugates, centrifuging the aggregates and removing the supernate containing un-conjugated protein A. Protein-polymer conjugation was confirmed by SDS-PAGE analysis. The polymer-protein A conjugate was temperature-responsive at a temperature threshold of ˜24° C.

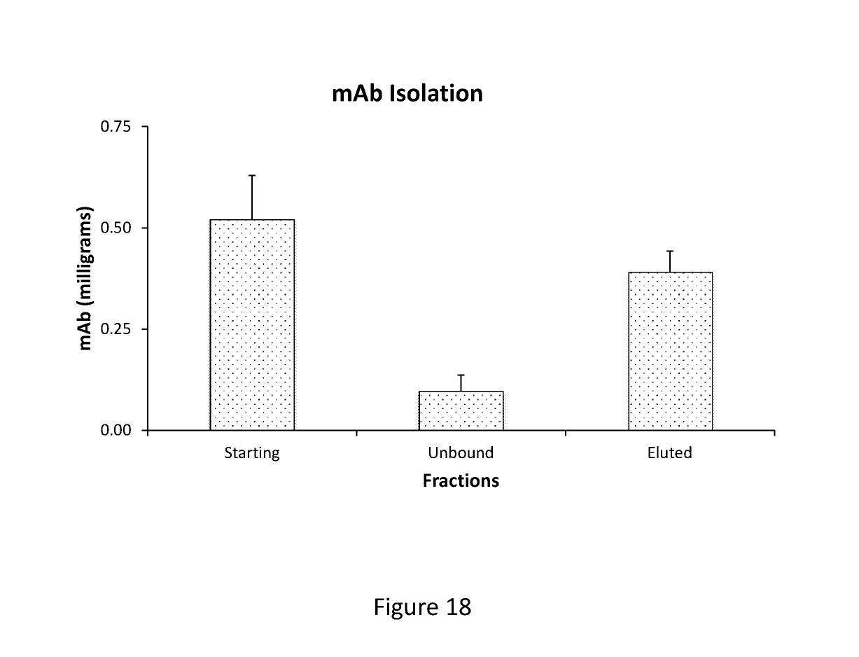

A starting solution of polymer-protein A conjugates and mAb (˜1 mg/mL) was mixed at 4° C. to allow soluble polymer-Protein A conjugates to bind to mAb. The starting solution was then heated to 24° C., and this thermal stimulus caused the polymer-protein A conjugates (with bound mAb) to co-aggregate. The solution was centrifuged to pellet the aggregated conjugates with bound mAb, and the supernate containing unbound mAb was collected. The pellet was then re-solubilized in an acidic elution buffer to elute the bound mAb from protein A. The elution buffer was then heated to 24° C. to aggregate the polymer-protein A conjugates (now without mAb), and centrifuged to precipitate the polymer-Protein A conjugates. The elution buffer supernatant containing freed mAb was collected. As is provided in FIG. 18, the mAb was quantified by UV-Vis spectroscopy (measuring absorbance at 280 nm) in the starting solution, the unbound fraction supernatant, and the elution buffer supernatant. The data demonstrate efficient capture of the mAb from solution, with the polymer-protein A conjugates, and subsequent elution of the bound mAb from the protein A.

### K. Example 10

### Long-Term Activation and Expansion of T Cells Using the Same Polymer-Affinity Reagent Conjugates

Polymer-anti-CD3 conjugates (as described in Examples 1-3) were added to purified human T cells on ice, which allowed the conjugates to bind to CD3 (part of the T cell receptor, or TCR) molecules on the T cell surfaces. The culture media was supplemented with 2% human AB serum, 1 μg/mL unconjugated anti-CD28 and 200 IU/mL interleukin-2 (IL-2). The cells were placed in a 37° C. incubator. The increase in temperature from ˜4° C. to 37° C. served as the stimulus for polymer-Ab conjugates to co-aggregate. Thus, the polymer-anti-CD3 conjugates co-aggregated at the cell surfaces and caused CD3 receptor clustering and cross-linking. T cell samples were collected for analysis every 2-3 days. Cell number and viability were assessed on an automated cell counting instrument (Nexcelom) according to the manufacturer's instructions for mean fold expansion calculations. Flow cytometry assessments of the percentage of viable cells expressing CD4+, CD8+ and CD25+ were performed on a BD LSR II Flow cytometer. Therefore, according to the subject embodiments, polymer-Ab conjugates are able to cross-link TCRs on T cell surfaces, leading to long-term T cell activation ex vivo in the presence of an additional soluble co-stimulatory signal.

The following provides a specific method of using two of the same polymer-affinity reagent conjugates to activate T cells ex vivo. The anti-CD3 Ab (clone OKT3) binds to the CD3 molecule associated with T cell receptors (TCRs). Agonistic anti-CD3 antibodies like clone OKT3 can engage the TCR but the antibody typically needs to be surface bound (i.e., microparticles) for TCR clustering and subsequent T cell activation. Even when TCRs are clustered, T cell activation can be muted without the presence of an additional, co-stimulatory signal. IL-2 and CD28 are examples of co-stimulatory molecules. IL-2 is a soluble cytokine involved in T cell differentiation, and it provides a co-stimulatory signal for T cell activation. CD28 is molecule on T cell surfaces that must be engaged by endogenous (B7 proteins) or other (anti-CD28 Ab, clone 9.3) molecules in conjunction with TCR engagement for maximal T cell activation. It can also be cross-linked with TCR.

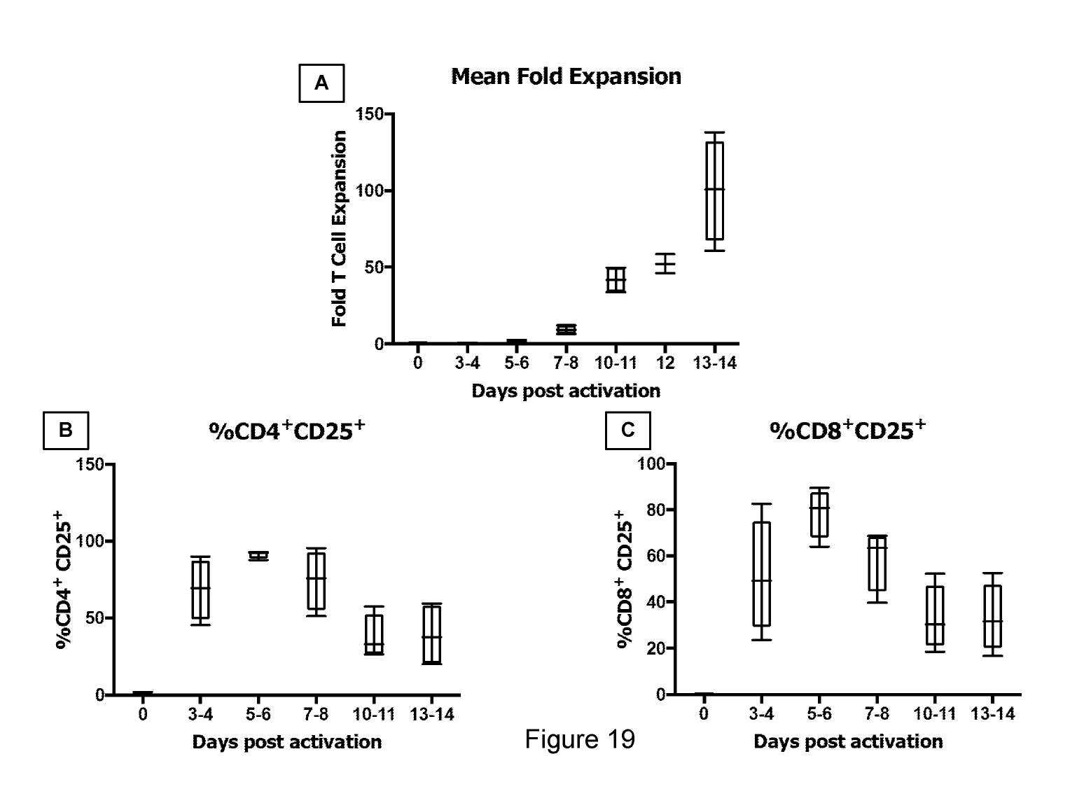

Temperature responsive polymers were conjugated to anti-CD3 Abs and analyzed as described in Examples 2 & 3. These polymer-anti-CD3 conjugates were added to purified human T cells (n=4 different donors) on ice, with IL-2 and anti-CD28. At this lower temperature, the conjugates bound to the CD3 molecules, IL-2 bound to IL-2 receptors and anti-CD28 mAb bound to CD28 on the T cell surfaces. The cells were placed in a 37° C. and incubator. The increase in temperature from ˜4° C. to 37° C. served as the stimulus for polymer-Ab conjugate co-aggregation. Thus, the polymer-anti-CD3 conjugates co-aggregated at the cell surfaces and caused CD3 receptor cross-linking. The data below demonstrated that the CD3 receptor cross-linking led to robust T cell activation, proliferation and expansion over two weeks. As is shown for example in FIG. 19A, cell numbers and viability were measured every 2-3 days, which showed that the T cells expanded after activation via CD3 receptor cross-linking. Cell surface markers (e.g., CD25) of T cell activation were also assessed via flow cytometry every 2-3 days. As is further shown for example in FIGS. 19B and 19C, both CD4+ and CD8+ T cells showed elevated levels of CD25 co-stimulation marker expression that were above background (day 0) and robust over two weeks in culture. Therefore, polymer-Ab conjugates were able to cross-link TCRs and induce T cell activation and expansion ex vivo, in the presence of an additional co-stimulatory signal.

### L. Example 11

### Preparation of Binding Entities Including a Plurality of Affinity Reagents Bound to a Stimuli-Responsive Polymer

Binding entities including a plurality of affinity reagents bound to a stimuli-responsive polymer (e.g., multivalent polymer-Ab conjugates) are made by following published protocols (Roy, et al., ACS Macro Letters 2:132-136 (2013)). First, a macro CTA is synthesized by RAFT polymerization of NIPAM, using a DP of 50, 100 or 200. This is followed by a RAFT block copolymerization of N,N-dimethylacrylamide (DMA) and an activated ester-containing monomer (e.g., acrylic acid N-hydroxysuccinimide ester (AANHS)). In the block copolymerization, the molar ratio of DMA to AANHS is varied in order to control both the stimuli-responsive behavior and the number of affinity reagents that can be conjugated to the polymer. Other co-monomers can also be used in the block copolymerization to modulate the stimuli-responsive behavior further (e.g., butyl acrylate). The resultant diblock copolymer, poly(NIPAM)-b-poly(DMA-co-AANHS) has a plurality of activated ester groups available for covalent conjugation to the affinity reagents.

A multivalent binding entity including a plurality of the same affinity reagents bound to a stimuli-responsive polymer is produced as follows: Monoclonal anti-CD3 (clone OKT-3) is diluted into sodium bicarbonate buffer (pH 9.5) and cooled. The Ab solution is added to poly(NIPAM)-b-poly(DMA-co-AANHS) and mixed for 18 hours at 4° C. Polymer:Ab molar ratios from 1:1 to 25:1 are explored. Free (non-polymer-bound) Ab is removed as in Example 2. The resultant binding entities with multiple anti-CD3 affinity reagents conjugated to the stimuli-responsive diblock copolymer are analyzed by SDS-PAGE and aqueous SEC. Binding curves of these binding entities in the presence of Jurkat cells are tested as in Example 3.

A multivalent binding entity including a plurality of different affinity reagents bound to a stimuli-responsive polymer is produced with similar techniques. Monoclonal anti-CD3 (clone OKT-3) and monoclonal anti-CD28 (clone 9.3) are diluted into sodium bicarbonate buffer (pH 9.5) and cooled, then added to poly(NIPAM)-b-poly(DMA-co-AANHS) and mixed for 18 hours at 4° C. The remaining steps in the protocol are as described above.

### M. Example 12

### Activation of Cells Using Binding Entities Including a Plurality of Affinity Reagents Bound to a Stimuli-Responsive Polymer

The use of binding entities including a plurality of different affinity reagents (i.e., anti-CD3 and anti-CD28) conjugated to a stimuli-responsive polymer is described first. These polymer-Ab conjugates are added to negatively-selected, purified human T cells on ice, which allows the soluble conjugates to bind to the CD3 (part of the T cell receptor, or TCR) and CD28 molecules on the T cell surfaces. The cells are then placed in a 37° C. incubator with 5% CO₂. The temperature increase from ˜4° C. to 37° C. serves as a stimulus for the stimuli-responsive polymer backbone to aggregate, thus co-localizing the anti-CD3 and anti-CD28 affinity reagents at the cell surfaces, causing CD3/CD28 receptor clustering. T cells are cultured in X-Vivo 15 medium containing 2% human AB sera, 200 IU/mL IL-2 and 1 ug/mL anti-CD28. Initial seeding densities are ˜1×10⁶ cells/mL and subsequent seeding densities are 5×10⁵ cells/mL. T cell proliferation is measured by CFSE dilution using flow cytometry after 5 days of culture. Otherwise, samples are collected for analysis every 2-3 days. Cell number, viability, and mean cell diameter are also assessed on an automated cell counting instrument according to the manufacturer's instructions. Flow cytometry assessments include the percentage of viable cells expressing the surface markers CD4, CD8, CD25, and CD69.

The use of binding entities including a plurality of the same affinity reagents (i.e., anti-CD3) bound to a stimuli-responsive polymer also can be applied to activate T cells. These polymer-Ab conjugates are added to purified human T cells on ice, which allows the soluble conjugates to bind to the CD3 molecules on the T cell surfaces. The cells are then placed in a 37° C. incubator with 5% CO₂. The temperature increase from ˜4° C. to 37° C. serves as a stimulus for the stimuli-responsive polymer backbone to aggregate, thus co-localizing the anti-CD3 affinity reagents at the cell surfaces, causing CD3 receptor clustering. T cell culture conditions and seeding densities are the same as described above. T cell proliferation is measured by CFSE dilution using flow cytometry after 5 days of culture. Otherwise, samples are collected for analysis every 2-3 days. Cell number, viability, and mean cell diameter are assessed on an automated cell counting instrument according to the manufacturer's instructions. Flow cytometry assessments include the percentage of viable cells expressing CD4, CD8, CD25, and CD69.

### N. Example 13

### Polymer-Conjugated Cytokines that Reversibly Control the Avidity of Ligand-Receptor Interactions

Cytokines (e. g., GM-CSF, IL-3) are important immune modulators and growth factors for diverse cell lineages in vivo and in vitro. Recombinant human GM-CSF is diluted into sterile PBS and cooled. The cytokine solution is added to poly(NIPAM₉-co-BA₁)-NHS and mixed for 18 hours at 4° C. The polymer: GM-CSF molar ratio for conjugations is varied from 1:1 to 50:1. Unconjugated GM-CSF is removed as described in Example 2. Polymer conjugation to the GM-CSF molecules is confirmed by SDS-PAGE analysis. The degree of conjugation is estimated via molecular weight calculations from aqueous SEC traces.

Binding curves of the polymer-GM-CSF conjugates and unconjugated GM-CSF are generated to assess the effect of polymer conjugation on the binding specificity of GM-CSF. Unconjugated GM-CSF or polymer- GM-CSF conjugates are added to TF-1 cells, at concentrations between 1-10 ng/mL. After binding for 25 minutes on ice, the cells are washed once to remove unbound GM-CSF. Then, mouse anti-human GM-CSF PE Ab is added to the cells for 20 minutes on ice to bind the GM-CSF or polymer-GM-CSF conjugates on the cell surfaces. After washing the cells to remove unbound secondary Ab, the cells are fixed and then analyzed for GM-CSF cell binding by flow cytometry.

Long-term culture of the cell line TF-1 (Kitamura, et al. Blood 73(1989) 375-380; Bittorf, et al. J Molecular Endocrinology 25(2000) 253-262) is dependent on the presence of certain cytokines in the growth media, including GM-CSF and/or IL-3. TF-1 cells are maintained in RPMI medium containing 10% FBS and 2 ng/mL GM-CSF. Before initiating experiments, the cells are briefly starved of serum and GM-CSF. Then, either unconjugated GM-CSF or polymer-GM-CSF conjugates (0-2 ng/mL GM-CSF equivalent) are added to TF-1 cells on ice, which allows the cytokines or conjugates to bind to the GM-CSF receptors (CD116) on the TF-1 cell surfaces. The cells are placed in a 37° C. incubator with 5% CO₂. The temperature increase from ˜4° C. to 37° C. serves as a stimulus for polymer-GM-CSF conjugates to co-aggregate at the cell surfaces, and causing CD116 receptor clustering. These polymer-mediated aggregates increase the effective local concentration of GM-CSF available to bind to the CD116 GM-CSF receptor. Then, if a single GM-CSF:CD116 interaction is disrupted, there are many other (aggregated) polymer-GM-CSF conjugates in close proximity to the receptor available for rapid re-engagement. Therefore, the avidity of the interaction is increased, and cell signaling is enhanced, even if the affinity of an individual ligand-receptor interaction remains relatively low. Every 24 hours for 7 days after adding the GM-CSF or polymer-GM-CSF conjugate, cell samples are removed from the culture and assayed for cell viability and cell number. Cell growth curves (cell number vs. time in culture, and t=7 day call number vs. GM-CSF concentration) are calculated in order to assess the impact of polymer-conjugated GM-CSF on TF-1 cell growth. WHO International Standard GM-CSF (NIBSC code 88/646) serves as a comparator in the growth curve assays. The iLite® GM-CSF Assay Ready cells are used as an alternative if TF-1 assays are difficult to interpret.

### O. Example 14

### Polymer-Affinity Reagent Conjugates to Cluster a Plurality of Either the Same or Different Cell Types

After binding to cell surface antigens, stimuli-responsive polymer-affinity reagent conjugates are used to cluster initially distant cell types. In separate conjugation reactions, monoclonal anti-CD19 (clone HIB19) and monoclonal anti-CD3 (clone OKT-3) are covalently linked to poly(NIPAM₉-co-BA₁)-NHS as described in Example 2. These reactions produce binding entities including affinity reagents (the mAbs) bound to one or more stimuli-responsive polymers. The purification and characterization of these mAb conjugates proceed as described in Examples 2 & 3. The polymer-anti-CD19 conjugates bind to CD19 antigens on B cell surfaces (e.g., the Raji cell line), and the polymer-anti-CD3 conjugates bind to CD3 antigens on T cell surfaces (e.g., the Jurkat cell line).

Raji and Jurkat cells (ATCC) are maintained independently in RPMI+10% FBS medium in a 37° C. incubator with 5% CO₂ atmosphere. Cells are passaged approximately 3 times per week at reseeding densities of 0.2×10⁶ cells/mL. On the day of experiments, the CD3+ Jurkat cells are labeled with freshly-made CFSE to aid in cell analysis. A mixture of CFSE-labeled Jurkat cells and unlabeled Raji cells is prepared by adding a volume of both cell suspensions to a low cell density (˜1×10⁴ cells/mL). A third, antigen-negative control cell type may also be added to the cell mixture. Unconjugated anti-CD19 and unconjugated anti-CD3 mAbs are incubated with the cells for 30 minutes at 4° C. Then, the cell mixture is warmed to 25° C. Cell counts and both bright-field and green fluorescent images of the cell mixture are obtained with a Nexcelom Cellometer. The numbers of adjacent Jurkat (green)/Jurkat (green), Jurkat (green)/Raji cells and Raji/Raji cells, in addition to the cell-cell distances, are determined using custom cell enumeration software and reported as a percentage of the total cell number. These data are the “no aggregation” control. Next, a Raji/Jurkat cell mixture (1×10⁴ cells/mL) is incubated with polymer-anti-CD19 and polymer-anti-CD3 conjugates at 4° C. for 30 minutes. At 4° C., the conjugates are soluble and able to bind their cognate cell surface antigens. Next, the polymer-mAb conjugates are aggregated by a temperature stimulus (an increase in temperature from ˜4° C. to 25° C.). The aggregation of the binding entities on CD19+ and CD3+ cell surface antigens clusters those cells. Thus, a higher percentage of the total cells are adjacent and cell-cell distances are smaller. Cell counts and both bright-field and green fluorescent images of the cell mixture are obtained and analyzed as described above.

### P. Example 15

### Polymer-Conjugated Small Molecules that Modulate Interactions at Cell Surface Receptors

Adjuvants (e. g., alum, squalene, CpG) are immune modulators that are added to vaccine formulations in order to boost and/or prolong a patient's immune response. Monophosphoryl Lipid A (MPLA) is an adjuvant and a ligand for the cell surface receptor Toll-Like Receptor 4 (TLR4). MPLA is a component of several FDA-approved vaccines like Cervarix™ and Fendrix™. MPLA binding stimulates the TLR4 receptor and leads to activation of generalized (i.e., not antigen-specific) “innate immunity,” including the generation of pro-inflammatory cytokines. As provided below, polymers according to the subject disclosure are conjugated to non-proteinaceous molecular entities, and the stimuli-responsive behavior of the subsequent conjugates is applied as an “on-off” switch for cell signaling.

MPLA is diluted into a mixed organic/aqueous solution and cooled. The MPLA solution is added to poly(NIPAM)-NHS and mixed for 18 hours at 4° C. The polymer:MPLA molar ratio for conjugations is varied from 1:1 to 50:1. Unconjugated MPL is removed as described in Example 2. The degree of polymer conjugation to MPLA molecules is calculated from NMR spectra and HPLC traces. The poly(NIPAM)-MPLA conjugates are expected to respond to temperature stimuli of ˜30° C.

Vials of iLite™ TLR4 Assay Ready Cells are purchased from EuroDiagnostica. These cells are derived from the K562 human erythroleukemia cell line and engineered to express firefly luciferase in response to TLR4 stimulation; luminescence can be quantified after adding a luciferase substrate. Either unconjugated MPLA or the polymer-MPLA conjugates (0-1000 ng/mL MPLA equivalent) are added to iLite™ TLR4 Assay Ready Cells at 25° C., which allows the adjuvant or conjugates to bind to TLR4 on the cell surfaces. Some cells are kept at room temperature, allowing the conjugates to remain soluble. Other cells are placed in a 37° C. incubator with 5% CO₂. The temperature increase from ˜25° C. to 37° C. serves as a stimulus for the polymer-MPLA conjugates to co-aggregate at the cell surfaces, and causing TLR4 clustering. These polymer-mediated aggregates should increase the effective local concentration of MPLA available to bind to the TLR4. Then, if a single MPLA:TLR4 interaction is disrupted, there are many other (aggregated) polymer-MPLA conjugates in close proximity to the receptor available for rapid re-engagement. Therefore, the avidity of the interaction is increased, and cell signaling is enhanced, even if the affinity of an individual ligand-receptor interaction remains relatively low. Approximately 5 hours after adding the MPLA or polymer-MPLA conjugates, the firefly luciferase substrate is added to the cells and the luminescence is read on a microplate reader. Concentration-response curves for unconjugated and conjugated MPLA are calculated at the two temperature conditions (25° C. and 37° C.) in order to assess the impact of polymer-conjugated MPLA on TLR4 signal transduction. Accordingly, the disclosed polymer-conjugated small molecules can control the presentation of a modulatory signal to cells in a stimuli-responsive manner.

In view of the above, methods and compositions for clustering cell surface molecules and their ligands are exemplified herein. The methods can include applying a stimulus to a polymer that is reversibly associative in response to the stimulus. The methods can also include activating cells and/or increasing signal transduction via cell surface molecules. While illustrative embodiments have been illustrated and described, it will be appreciated that various changes can be made therein without departing from the spirit and scope of the disclosure.

### VII. REFERENCES

References as set forth in the subject disclosure and which are each incorporated by reference in their entirety for all purposes include each of the publications provided below.

Kitamura T, Tojo A, Kuwaki T, Chiba S, Miyazono K, Urabe A, Takaku F. Identification and analysis of human erythropoietin receptors on a factor-dependent cell line, TF-1. Blood 73 (1989) 375-380.

T Bittorf, R Jaster, M J Soares, J Seiler, J Brock, K Friese and H Müller. Induction of erythroid proliferation and differentiation by a trophoblast-specific cytokine involves activation of the JAK/STAT pathway. Journal of Molecular Endocrinology 25 (2000) 253-262.

Kawa Amin. The role of mast cells in allergic inflammation. Respiratory Medicine (2012) 106, 9-14.

Laurence Besnault, Nicolas Schrantz, Marie Thérèse Auffredou, Gérald Leca, Marie Françoise Bourgeade and Aimé Vazquez. B Cell Receptor Cross-Linking Triggers a Caspase-8-Dependent Apoptotic Pathway That Is Independent of the Death Effector Domain of Fas-Associated Death Domain Protein. The Journal of Immunology 167 (2001) 733-740.

Maria-Ana Ghetie, Helen Bright, and Ellen S. Vitetta. Homodimers but not monomers of Rituxan (chimeric anti-CD20) induce apoptosis in human B-lymphoma cells and synergize with a chemotherapeutic agent and an immunotoxin. Blood 97 (2001) 1392-1398.

Julie P. Deans, Haidong Li, Maria J. Polyak. CD20-mediated apoptosis: signalling through lipid rafts. Immunology 107 (2002) 176-182.

Arash Grakoui, Shannon K. Bromley, Cenk Sumen, Mark M. Davis, Andrey S. Shaw, Paul M. Allen, Michael L. Dustin. The Immunological Synapse: A Molecular Machine Controlling T Cell Activation. Science 285 (1999) 221-227.

Matthias Gunzer, Carsten Weishaupt, Anja Hillmer, Yasmin Basoglu, Peter Friedl, Kurt E. Dittmar, Waldemar Kolanus, Georg Varga, and Stephan Grabbe. A spectrum of biophysical interaction modes between T cells and different antigen-presenting cells during priming in 3-D collagen and in vivo. Blood 104 (2004) 2801-2809.

M. Jules Mattes, Rosana B. Michel, David M. Goldenberg, and Robert M. Sharkey. Induction of Apoptosis by Cross-Linking Antibodies Bound to Human B-Lymphoma Cells: Expression of Annexin V Binding Sites on the Antibody Cap. Cancer Biotherapy and Radiopharmaceuticals 24 (2009) 185-193.

Robert H. Vonderheide, Martin J. Glennie. Agonistic CD40 Antibodies and Cancer Therapy. Clinical Cancer Research 19 (2013) 1035-1043.

Caitlin E. Sedwick, Margaret M. Morgan, Lismaida Jusino, Judy L. Cannon, Jim Miller and Janis K. Burkhardt. TCR, LFA-1, and CD28 Play Unique and Complementary Roles in Signaling T Cell Cytoskeletal Reorganization. Journal of Immunology 162 (1999) 1367-1375.

Theoharis C. Theoharides, Konstantinos-Dionysios Alysandratos, Asimenia Angelidou, Danae-Anastasia Delivanis, Nikolaos Sismanopoulos, Bodi Zhang, Shahrzad Asadi, Magdalini Vasiadi, Zuyi Weng, Alexandra Miniati, Dimitrios Kalogeromitros. Mast cells and inflammation. Biochimica et Biophysica Acta 1822 (2012) 21-33.

K Mimori, N Kiyokawa, T Taguchi, T Suzuki, T Sekino, H Nakajima, M Saito, Y U Katagiri, K Isoyama, K Yamada, Y Matsuo and J Fujimoto. Costimulatory signals distinctively affect CD20- and B-cell-antigen-receptor-mediated apoptosis in Burkitt's lymphoma/leukemia cells. Leukemia 17 (2003) 1164-1174.

Lee P. Richman and Robert H. Vonderheide. Role of Crosslinking for Agonistic CD40 Monoclonal Antibodies as Immune Therapy of Cancer. Cancer Immunology Research 2 (2014) 19-26.

Annette Trickett, Yiu Lam Kwan. T cell stimulation and expansion using anti-CD3/CD28 beads. Journal of Immunological Methods 275 (2003) 251-255.

Marcus O. Butler, Naoto Hirano. Human cell-based artificial antigen-presenting cells for cancer immunotherapy. Immunological Reviews 257 (2014) 191-209.

Joel C. Sunshine, Karlo Perica, Jonathan P. Schneck, Jordan J. Green. Particle shape dependence of CD8 [symbol] T cell activation by artificial antigen presenting cells. Biomaterials 35 (2014) 269-277.

Erin R. Steenblock, Tarek Fadel, Michael Labowsky, Jordan S. Pober, and Tarek M. Fahmy. An Artificial Antigen-presenting Cell with Paracrine Delivery of IL-2 Impacts the Magnitude and Direction of the T Cell Response. The Journal of Biological Chemistry 286 (2011) 34883-34892.

David L. Porter, Bruce L. Levine, Nancy Bunin, Edward A. Stadtmauer, Selina M. Luger, Steven Goldstein, Alison Loren, Julie Phillips, Sunita Nasta, Alexander Perl, Steven Schuster, Donald Tsai, Ambika Sohal, Elizabeth Veloso, Stephen Emerson, and Carl H. June. A phase 1 trial of donor lymphocyte infusions expanded and activated ex vivo via CD3/CD28 costimulation. Blood 107 (2006) 1325-1331.

Heiko Bruns, Catherine Bessell, Juan Carlos Varela, Carl Haupt, Jerry Fang, Shirin Pasemann, Andreas Mackensen, Mathias Oelke, Jonathan P. Schneck, and Christian Schutz. CD47 Enhances In Vivo Functionality of Artificial Antigen-Presenting Cells. Clinical Cancer Research 21 (2015) 2075-2083.

Karlo Perica, Joan Glick Bieler, Christian Schutz, Juan Carlos Varela, Jacqueline Douglass, Andrew Skora, Yen Ling Chiu, Mathias Oelke, Kenneth Kinzler, Shibin Zhou, Bert Vogelstein, and Jonathan P. Schneck. Enrichment and Expansion with Nanoscale Artificial Antigen Presenting Cells for Adoptive Immunotherapy. ACS Nano 9 (2015) 6861-6871.

Forget M A, Malu S, Liu H, Toth C, Maiti S, Kale C, Haymaker C, Bernatchez C, Huls H, Wang E, Marincola F M, Hwu P, Cooper L J, Radvanyi L G. Activation and propagation of tumor-infiltrating lymphocytes on clinical-grade designer artificial antigen-presenting cells for adoptive immunotherapy of melanoma. Journal of Immunotherapy 37 (2014) 448-60.

Michael C Jensen, Stanley R Riddell. Designing chimeric antigen receptors to effectively and safely target tumors. Current Opinion in Immunology 33 (2015) 9-15.

Marcela V. Maus, Anna K. Thomas, Debra G. B. Leonard, David Allman, Kathakali Addya, Katia Schlienger, James L. Riley, and Carl H. June. Ex vivo expansion of polyclonal and antigen-specific cytotoxic T lymphocytes by artificial APCs expressing ligands for the T-cell receptor, CD28 and 4-1BB. Nature Biotechnology 20 (2002) 143-148.

A D Kaiser, M Assenmacher, B Schröder, M Meyer, R Orentas, U Bethke, B Dropulic. Towards a commercial process for the manufacture of genetically modified T cells for therapy. Cancer Gene Therapy 22 (2015) 72-78.

D. Teschner, G. Wenzel, E. Distler, E. Schnurer, M. Theobald, A. A. Neurauter, K. Schjetne, W. Herr. In Vitro Stimulation and Expansion of Human Tumour-Reactive CD8+ Cytotoxic T Lymphocytes by Anti-CD3/CD28/CD137 Magnetic Beads. Scandinavian Journal of Immunology 74 (2011) 155-164.

Subhra Mandal, Zaskia H. Eksteen-Akeroyd, Monique J. Jacobs, Roel Hammink, Matthieu Koepf, Annechien J. A. Lambeck, Jan C. M. van Hest, Christopher J. Wilson, Kerstin Blank, Carl G. Figdor and Alan E. Rowan. Therapeutic nanoworms: towards novel synthetic dendritic cells for immunotherapy. Chemical Science 4 (2013) 4168-4174.

Levine B L, Bernstein W B, Connors M, Craighead N, Lindsten T, Thompson C B, June C H. Effects of CD28 costimulation on long-term proliferation of CD4+ T cells in the absence of exogenous feeder cells. Journal of Immunology 159 (1997) 5921-5930.

Levine B. L., Mosca J. D., Riley J. L. et al. Antiviral effect and ex vivo CD4⁺ T cell proliferation in HIV-positive patients as a result of CD28 costimulation. Science 272 (1996) 1939-43.

Lamers C. H., van de Griend R. J., Braakman E. et al. Optimization of culture conditions for activation and large-scale expansion of human T lymphocytes for bispecific antibody-directed cellular immunotherapy. Int J Cancer 51 (1992) 973-9.

Turka L A, Ledbetter J A, Lee K, June C H, Thompson C B. CD28 is an inducible T cell surface antigen that transduces a proliferative signal in CD3+ mature thymocytes. Journal of Immunology 144 (1990) 1646-53.

Riddell, S. R., P. D. Greenberg. The use of anti-CD3 and anti-CD28 monoclonal antibodies to clone and expand human antigen-specific T cells. Journal of Immunological Methods 128 (1990) 189-201.

Steven A. Rosenberg, M.D., Ph.D., Beverly S. Packard, Ph.D., Paul M. Aebersold, Ph.D., Diane Solomon, M.D., Suzanne L. Topalian, M.D., Stephen T. Toy, Ph.D., Paul Simon, Ph.D., Michael T. Lotze, M.D., James C. Yang, M.D., Claudia A. Seipp, R.N., Colleen Simpson, R.N., Charles Carter, Steven Bock, M.D., Douglas Schwartzentruber, M.D., John P. Wei, M.D., and Donald E. White, M.S. Use of Tumor-Infiltrating Lymphocytes and Interleukin-2 in the Immunotherapy of Patients with Metastatic Melanoma. New England Journal of Medicine 319 (1988) 1676-1680.

Dixon J F, Law J L, Favero J J. Activation of human T lymphocytes by crosslinking of anti-CD3 monoclonal antibodies. Journal of Leukocyte Biology 46 (1989) 214-20.

Loek J. Eggermont, Leonie E. Paulis, Jurjen Tel, and Carl G. Figdor. Towards efficient cancer immunotherapy: advances in developing artificial antigen-presenting cells. Trends in Biotechnology 32 (2014) 456-465.

Damir Vidovic, Thomas J. Graddis, Lara P. Stepan, Dennis M. Zaller, and Reiner Laus. Specific Stimulation of MHC-Transgenic Mouse T-Cell Hybridomas With Xenogeneic APC. Human Immunology 64 (2003) 238-244.

David H. Canaday, Adam Gehring, Ethan G. Leonard, Brandon Eilertson, John R. Schreiber, Clifford V. Harding, W. Henry Booma. T-cell hybridomas from HLA-transgenic mice as tools for analysis of human antigen processing. Journal of Immunological Methods 281 (2003) 129-142.

Cluff, C. W. Monophosphoryl Lipid A (MPL) as an Adjuvant for Anti-Cancer Vaccines: Clinical Results. Lipid A in Cancer Therapy, J-F Jeannin, ed. 2009 Landes Bioscience.

Bhardwaj, N., S. Gnjatic, N. B. Sawhney. TLR Agonists: Are They Good Adjuvants?. Cancer J. 16 (2010) 382-391.

Kolanowski S T, Lissenberg-Thunnissen S N, Emal D, van Ham S M, Ten Brinke A. Monophosphoryl lipid A-induced pro-inflammatory cytokine expression does not require CD14 in primary human dendritic cells. Inflamm Res. 65 (2016) 449-58.

Michaud J-P, Hallé M, Lampron A, Thériault P, Préfontaine P, Filali M, Tribout-Jover P, Lanteigne A-M, Jodoin R, Cluff C, Brichard V, Palmantier R, Pilorget A, Larocque D, Rivest S. Toll-like receptor 4 stimulation with the detoxified ligand monophosphoryl lipid A improves Alzheimer's disease-related pathology. PNAS 110 (2013) 1941-1946.

Roy D, Nehilla B J, Lai J J, Stayton P S. Stimuli-Responsive Polymer-Antibody Conjugates via RAFT and Tetrafluorophenyl Active Ester Chemistry. ACS Macro Letters 2 (2013) 132-136.

Although the foregoing embodiments of the invention have been described in some detail by way of illustration and example for purposes of clarity of understanding, it is readily apparent to those of ordinary skill in the art in light of the teachings of this subject matter that certain changes and modifications may be made thereto without departing from the spirit or scope of the appended claims. It is also to be understood that the terminology used herein is for the purpose of describing particular embodiments only, and is not intended to be limiting, since the scope of the present embodiments of the invention will be limited only by the appended claims.

Accordingly, the preceding merely illustrates the principles of the embodiments of the invention. It will be appreciated that those skilled in the art will be able to devise various arrangements which, although not explicitly described or shown herein, embody the principles of the embodiments of the invention and are included within its spirit and scope. Furthermore, all examples and conditional language recited herein are principally intended to aid the reader in understanding the principles of the embodiments of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, e.g., any elements developed that perform the same function, regardless of structure. The scope of the presently disclosed subject matter, therefore, is not intended to be limited to the exemplary embodiments shown and described herein. Rather, the scope and spirit of presently disclosed subject matter is embodied by the appended claims.

What is claimed is:

1. A method of clustering cell surface molecules, the method comprising:
a. contacting a cell with a plurality of binding entities each comprising an affinity reagent bound to one or more polymers that are reversibly associative in response to a stimulus,
i. wherein the cell comprises the cell surface molecules on its surface and contacting the cell with the binding entities includes binding a plurality of the affinity reagents to the cell surface molecules; and
b. applying the stimulus to associate at least some of the plurality of binding entities to one another and thereby clustering the cell surface molecules bound to affinity reagents.

2. The method according to claim 1, wherein the stimulus is a change in a condition selected from the group consisting of temperature, pH, light, photo-irradiation, exposure to an electric field, ionic strength, concentration of a specified anion concentration of a specified cation, and combinations thereof.

3. The method according to claim 1 or claim 2, wherein the plurality of binding entities comprises a first affinity reagent and a second affinity reagent different than the first affinity reagent; wherein the cell surface molecules comprise a first cell surface molecule and a second cell surface molecule different than the first cell surface molecule, and wherein binding the affinity reagents to the cell surface molecules comprises binding the first affinity reagent to the first cell surface molecule and binding the second affinity reagent to the second cell surface molecule.

4. The method according to any one of claims 1-3, further comprising: contacting the cell with a plurality of stimuli-responsive magnetic nanoparticles each comprising at least one of the one or more polymers that are reversibly associative in response to a stimulus.

5. The method according to any one of claims 3-4, wherein the first and second cell surface molecules comprise first and second cell surface receptors.

6. The method according to claim 5, wherein the first and second cell surface receptors comprise receptors selected from the group consisting of T cell receptor, human leukocyte antigen (HLA), B cell receptor, major histocompatibility complex (MHC), FcεR1, CD2, CD3, CD4, CD8, CD14, CD19, CD 20, CD28, CD30, CD 40, CD45, CD116, CD152 and CD169, and other receptors for cytokines, chemokines, hormones, and growth factors.

7. The method according to claim 6, wherein the first cell surface receptor is selected from the group consisting of T cell receptors CD3 and CD28.

8. The method according to claim 6, wherein the second cell surface receptor is selected from the group consisting of T cell receptors CD3 and CD28.

9. The method according to claim 6, wherein the first cell surface receptor is CD3 and the second cell surface receptor is CD3.

10. The method according to claim 6, wherein the first cell surface receptor is CD28 and the second cell surface receptor is CD28.

11. The method according to claim 6, wherein the first cell surface receptor is CD3 and the second cell surface receptor is CD28.

12. The method according to any one of claims 1-11, wherein the polymer comprises a homopolymer or a copolymer.

13. The method according to any one of claims 1-12, wherein the polymer comprises a diblock copolymer, a triblock copolymer, a graft copolymer, or a brush copolymer.

14. The method according to any one of claims 1-13, wherein the polymer comprises poly(N-isopropylacrylamide).

15. The method according to any one of claims 1-14, wherein clustering the cell surface molecules bound to affinity reagents promotes activating the cell.

16. The method according to any one of claims 1-15, wherein clustering the cell surface molecules bound to affinity reagents promotes increasing signal transduction via the cell surface molecules.

17. The method according to claim 4, further comprising isolating the cell from one or more other cells by applying a magnetic field to move the cell-bound stimuli-responsive magnetic nanoparticle in the field.

18. The method according to any one of claims 1-3 or 5-17, further comprising: contacting a binding entity bound to one or more of the cell surface molecules with one or more additional polymers and thereby binding the additional polymers to the binding entity.

19. The method according to any one of claims 1-13 or 15-18, wherein the polymer comprises poly(N-isopropylacrylamide-co-butyl acrylate).

20. A method of clustering cell surface molecules, the method comprising:
a. contacting a cell with a binding entity comprising a plurality of affinity reagents bound to a polymer that is reversibly associative in response to a stimulus,
i. wherein the cell comprises the cell surface molecules on its surface and contacting the cell with the binding entity includes binding a plurality of the affinity reagents to the cell surface molecules; and
b. applying the stimulus to associate at least some of the plurality of affinity reagents to one another and thereby clustering the cell surface molecules bound to affinity reagents.

21. The method according to claim 20, wherein the binding entity comprises a first affinity reagent and a second affinity reagent different than the first affinity reagent; wherein the cell surface molecules comprise a first cell surface molecule and a second cell surface molecule different than the first cell surface molecule, and wherein binding the affinity reagents to the cell surface molecules comprises binding the first affinity reagent to the first cell surface molecule and binding the second affinity reagent to the second cell surface molecule.

22. The method according to claim 20 or claim 21, wherein the stimulus is a change in a condition selected from the group consisting of temperature, pH, light, photo-irradiation, exposure to an electric field, ionic strength, concentration of a specified anion, concentration of a specified cation, and combinations thereof.

23. The method according to any one of claims 20-22, further comprising: contacting the cell with a plurality of stimuli-responsive magnetic nanoparticles each comprising at least one of the one or more polymers that are reversibly associative in response to a stimulus.

24. The method according to any one of claims 20-23, wherein the cell surface molecules comprise first and second cell surface receptors.

25. The method according to claim 24, wherein the first and second cell surface receptors comprise receptors selected from the group consisting of T cell receptor, human leukocyte antigen (HLA), B cell receptor, major histocompatibility complex (MHC), FcεR1, CD2, CD3, CD4, CD8, CD14, CD19, CD 20, CD28, CD30, CD45, CD116, CD152, and CD169, and other receptors for cytokines, chemokines, hormones, and growth factors.

26. The method according to claim 25, wherein the first cell surface receptor is selected from the group consisting of T cell surface receptors, CD3 and CD28.

27. The method according to claim 25, wherein the second cell surface receptor is selected from the group consisting of T cell surface receptors CD3 and CD28.

28. The method according to claim 25, wherein the first cell surface receptor is CD3 and the second cell surface receptor is CD3.

29. The method according to claim 25, wherein the first cell surface receptor is CD28 and the second cell surface receptor is CD28.

30. The method according to claim 25, wherein the first cell surface receptor is CD3 and the second cell surface receptor is CD28.

31. The method according to any one of claims 20-30, wherein the polymer is a homopolymer or a copolymer.

32. The method according to any one of claims 20-31, wherein the polymer is a diblock copolymer, a triblock copolymer, or a graft copolymer.

33. The method according to any one of claims 20-32, wherein the polymer comprises poly(N-isopropylacrylamide).

34. The method according to any one of claims 19-32, wherein the polymer comprises poly(N-isopropylacrylamide-co-butyl acrylate).

35. The method according to any one of claims 18-32, wherein clustering cell surface molecules bound to affinity reagents promotes activating the cell.

36. The method according to any one of claims 18-33, wherein clustering cell surface molecules bound to affinity reagents promotes increasing signal transduction via the cell surface molecules.

37. The method according to claim 23, further comprising isolating the cell from one or more other cells by applying a magnetic field to move the cell-bound stimuli-responsive magnetic nanoparticle in the field.

38. The method according to any one of claims 19-21 or 23-36, further comprising: contacting a binding entity bound to one or more of the cell surface molecules with one or more additional polymers and thereby binding the additional polymers to one or more binding entities.

39. A stimulus-responsive reagent, comprising:
a binding entity comprising a plurality of affinity reagents bound to a polymer that is reversibly associative in response to a stimulus.

40. The reagent according to claim 39, wherein said plurality of affinity reagents comprises antibodies, antibody fragments, scFvs or other antigen-binding molecules.

41. The reagent according to claim 39 or 40, wherein the plurality of affinity reagents comprises a first affinity reagent and a second affinity reagent different than the first affinity reagent.

42. The reagent according to claims 39 or 40, wherein the plurality of affinity reagents comprises a first affinity reagent and a second affinity reagent of the same type as the first affinity reagent.

43. The reagent according any one of claims 39-42, wherein the affinity reagents are capable of binding to cell surface molecules when the binding entities are contacted with a cell comprising the cell surface molecules, and whereupon application of the stimulus the affinity reagents are capable of associating at least some of the plurality of affinity reagents to one another.

44. The reagent of claim 43, wherein the cell surface molecules comprise cell surface receptors.

45. The reagent according to claim 43 or claim 44, wherein the cell surface receptors comprise receptors selected from the group consisting of T cell receptor, human leukocyte antigen (HLA), B cell receptor, major histocompatibility complex (MHC), FcεR1, CD2, CD3, CD4, CD8, CD14, CD19, CD 20, CD28, CD30, CD 40, CD45, CD116, CD152 and CD169, and other receptors for cytokines, chemokines, hormones, and growth factors.

46. The reagent according to claims 44 or 45, wherein the cell surface receptors comprise a first cell surface receptor and a second cell surface receptor, and wherein the first cell surface receptor is selected from the group consisting of T cell surface receptors CD3 and CD28.

47. The reagent according to any one of claims 44-46, wherein the cell surface receptors comprise a first cell surface receptor and a second cell surface receptor, and wherein the second cell surface receptor is selected from the group consisting of T cell receptors CD3 and CD28.

48. The reagent according to any one of claims 44-47, wherein the cell surface receptors comprise a first cell surface receptor and a second cell surface receptor, and wherein the first cell surface receptor is CD3 and the second cell surface receptor is CD3.

49. The reagent according to any one of claims 44-46, wherein the cell surface receptors comprise a first cell surface receptor and a second cell surface receptor, and wherein the first cell surface receptor is CD28 and the second cell surface receptor is CD28.

50. The reagent according to any one of claims 44-46, wherein the cell surface receptors comprise a first cell surface receptor and a second cell surface receptor, and wherein the first cell surface receptor is CD3 and the second cell surface receptor is CD28.

51. The reagent according to any one of claims 39-50, wherein the stimulus is a change in a condition selected from the group consisting of temperature, pH, light, photo-irradiation, exposure to an electric field, ionic strength, concentration of a specified anion, concentration of a specified cation, and combinations thereof.

52. The reagent according to any one of claims 39-51, wherein the binding entity further comprises one or more of the plurality of affinity reagents bound to a plurality of stimuli-responsive magnetic nanoparticles comprising at least one of the one or more polymers that are reversibly associative in response to a stimulus.

53. The reagent according to any one of claims 39-52, wherein the polymer is a homopolymer or a copolymer.

54. The reagent according to any one of claims 39-53, wherein the polymer is a diblock copolymer, a triblock copolymer, or a graft copolymer.

55. The reagent according to any one of claims 39-54, wherein the polymer comprises poly(N-isopropylacrylamide).

56. The reagent according to any one of claims 39-54, wherein the polymer comprises poly(N-isopropylacrylamide-co-butyl acrylate).

57. A kit for clustering cell surface molecules, the kit comprising:
a. a first composition comprising:
i. a first binding entity comprising one or more first affinity reagents bound to a first polymer that is reversibly associative in response to a first stimulus; and
b. a second composition comprising:
i. a second binding entity comprising one or more second affinity reagents bound to a second polymer that is reversibly associative in response to a second stimulus.

58. The kit of claim 57, further comprising containers for containing said first and second compositions and instructions for contacting a cell with said first and second compositions and applying first and second stimuli to effect clustering of cell surface molecules by associating said first and second polymers responsive to said first and second stimuli applications.

59. The kit according to any one of claims 57-58, wherein said first and second compositions and said first and second stimuli are the same.

60. The kit according to any one of claims 57-58, wherein said first affinity reagent and said second affinity reagent are different.

61. The kit according to any one of claims 57-60, whereupon application of the first and second stimuli causes association of the first binding entity of the first composition to the second binding entity of the second composition, and clustering of the cell surface molecules bound to the first affinity reagents and the second affinity reagents.

62. The kit of claim 61, wherein the cell surface molecules comprise cell surface receptors.

63. The kit according to claim 61 or claim 62, wherein the cell surface receptors comprise receptors selected from the group consisting of T cell receptor, human leukocyte antigen (HLA), B cell receptor, major histocompatibility complex (MHC), FcεR1, CD2, CD3, CD4, CD8, CD14, CD19, CD 20, CD28, CD30, CD 40, CD45, CD116, CD152 and CD169, and other receptors for cytokines, chemokines, hormones, and growth factors.

64. The kit according to any one of claims 57-63, wherein the stimulus is a change in a condition selected from the group consisting of temperature, pH, light, photo-irradiation, exposure to an electric field, ionic strength, specific anions, specific cations, and combinations thereof.

65. The kit according to any one of claims 57-64, wherein each binding entity further comprises one or more stimuli-responsive magnetic nanoparticles comprised of at least one of the one or more polymers that are reversibly associative in response to a stimulus.

66. The kit according to any one of claims 57-64, wherein each binding entity further comprises one or more additional polymers that are reversibly associative in response to a stimulus thereby binding the additional polymers to the binding entity.

67. The kit according to any one of claims 57-65, wherein the polymer is a homopolymer or a copolymer.

68. The kit according to any one of claims 57-67, wherein the polymer is a diblock copolymer, a triblock copolymer, a graft copolymer, or a brush copolymer.

69. The kit according to any one of claims 57-68, wherein the polymer comprises poly(N-isopropylacrylamide).

70. The kit according to any one of claims 57-68, wherein the polymer comprises poly(N-isopropylacrylamide-co-butyl acrylate).

71. The kit according to any one of claims 57-70, wherein said first binding entity comprises a plurality of first affinity reagents.

72. The kit according to any one of claims 57-71, wherein said second binding entity comprises a plurality of second affinity reagents.

73. A method of co-localizing cells, the method comprising:
a. contacting a first cell with a plurality of binding entities each comprising an affinity reagent bound to one or more polymers that are reversibly associative in response to a stimulus,
i. wherein the first cell comprises one or more cell surface molecules on its surface and contacting the first cell with the binding entities comprises binding one or more of the affinity reagents to the one or more cell surface molecules;
b. contacting a second cell with a plurality of binding entities each comprising an affinity reagent bound to one or more polymers that are reversibly associative in response to a stimulus,
i. wherein the second cell comprises one or more cell surface molecules on its surface and contacting the second cell with the binding entities comprises binding one or more of the affinity reagents to the one or more cell surface molecules; and
c. applying the stimulus to associate at least one of the binding entities bound to the first cell and at least one of the binding entities bound to the second cell to one another and thereby co-localizing the first and second cells.

74. The method according to claim 73, wherein the first cell has a first cell type and the second cell has a second cell type which is different than the first cell type.

75. The method according to claim 73, wherein the first cell has a first cell type and the second cell has a second cell type which is the same as the first cell type.

76. The method according to claim 75, wherein the first cell is a T cell and the second cell is an antigen presenting cell.

77. The method according to any one of claims 73-75, wherein the one or more cell surface molecules of the first cell are of a same type as the one or more cell surface molecules of the second cell.

78. The method according to any one of claims 73-75, wherein the one or more cell surface molecules of the first cell are of a different type than the one or more cell surface molecules of the second cell.

79. The method according to any one of claims 73-78, wherein the one or more cell surface molecules of the first cell and the one or more cell surface molecules of the second cell are cell surface receptors.

80. The method according to claim 79, wherein the cell surface receptors comprise receptors selected from the group consisting of T cell receptor, human leukocyte antigen (HLA), B cell receptor, major histocompatibility complex (MHC), FcεR1, CD2, CD3, CD4, CD8, CD14, CD19, CD20, CD28, CD30, CD45, CD116, CD152, CD169, and other receptors for cytokines, chemokines, hormones, and growth factors

81. The method according to claim 79, wherein the one or more cell surface molecules of the first cell are selected from the group consisting of T cell surface receptors CD3 and CD28.

82. The method according to claim 79, wherein the one or more cell surface molecules of the second cell are selected from the group consisting of T cell surface receptors CD3 and CD28.

83. The method according to claim 79, wherein the one or more cell surface molecules of the second cell are not selected from the group consisting of T cell surface receptors CD3 and CD28.

84. The method according to any one of claims 73-82, wherein at least one of the binding entities bound to the first cell and the at least one of the binding entities bound to the second cell are of different types.

85. The method according to any one of claims 73-84, wherein each binding entity further comprises one or more stimuli-responsive magnetic nanoparticles comprised of at least one of the one or more polymers that are reversibly associative in response to a stimulus.

86. The method according to any one of claims 73-85, wherein each binding entity comprises two or more polymers which are reversibly associative in response to the stimulus.

87. A method of co-localizing cells, the method comprising:
a. contacting a first cell with a first binding entity comprising a plurality of first affinity reagents bound to a first polymer that is reversibly associative in response to a first stimulus,
i. wherein the first cell comprises first cell surface molecules on its surface and contacting the first cell with the first binding entity comprises binding the plurality of the first affinity reagents to the first cell surface molecules;
b. contacting a second cell with a second binding entity comprising a plurality of second affinity reagents bound to a second polymer that is reversibly associative in response to a second stimulus,
i. wherein the second cell comprises second cell surface molecules on its surface and contacting the second cell with the second binding entity comprises binding the plurality of the second affinity reagents to the second cell surface molecules; and
c. applying the first and the second stimuli to associate the first binding entity to the second binding entity and thereby co-localizing the first and second cells.

88. The method according to claim 87, wherein the first binding entity and the second binding entity are the same.

89. The method according to claim 87, wherein the first binding entity and the second binding entity are different.

90. The method according to any one of claims 87-88, wherein the first and the second stimuli are the same.

91. The method according to any one of claims 87 and 89, wherein the first and the second stimuli are different.

92. The method according to claim 87, wherein the first cell has a first cell type and the second cell has a second cell type which is the same as the first cell type.

93. The method according to claim 87, wherein the first cell has a first cell type and the second cell has a second cell type which is different from the first cell type.

94. The method according to claim 93, wherein the first cell is a T cell and the second cell is an antigen presenting cell.

95. The method according to any one of claims 87-92, and wherein the cell surface molecules of the first cell are of a same type as the cell surface molecules of the second cell.

96. The method according to any one of claims 87-95, wherein the cell surface molecules of the first cell are of different types.

97. The method according to any one of claims 87-96, wherein the cell surface molecules of the first cell are of a different type as the cell surface molecules of the second cell.

98. The method according to any one of claims 87-97, the cell surface molecules of the second cell are of different types.

99. The method according to any one of claims 87-98, wherein the cell surface molecules of the first cell and the cell surface molecules of the second cell are cell surface receptors.

100. The method according to claim 99, wherein the cell surface receptors comprise receptors selected from the group consisting of T cell receptor, human leukocyte antigen (HLA), B cell receptor, major histocompatibility complex (MHC), FcεR1, CD2, CD3, CD4, CD8, CD19, CD28, CD 30, CD45, CD116, CD152 and CD169, and other receptors for cytokines, chemokines, hormones, and growth factors.

101. The method according to claim 100, wherein the cell surface molecules of the first cell are selected from the group consisting of T cell receptors CD3 and CD28.

102. The method according to claim 100, wherein the cell surface molecules of the second cell are selected from the group consisting of T cell receptors CD3 and CD28.

103. The method according to any one of claims 87-102, wherein the at least one of the binding entities bound to the first cell and the at least one of the binding entities bound to the second cell are of different types.

104. The method according to any one of claims 87-103, wherein each binding entity further comprises one or more stimuli-responsive magnetic nanoparticles comprised of at least one of the one or more polymers that are reversibly associative in response to a stimulus.

105. The method according to any one of claims 87-104, wherein each binding entity comprises one or more additional soluble polymers which are reversibly associative in response to the stimulus.

106. A method of clustering cell surface molecules, the method comprising:
a. contacting a cell with a plurality of first binding entities each comprising a first affinity reagent bound to one or more first polymers that are reversibly associative in response to a first stimulus,
i. wherein the cell comprises first cell surface molecules on its surface and contacting the cell with the first binding entities comprises binding a plurality of the first affinity reagents to the cell surface molecules;
b. applying a first stimulus to associate at least some of the plurality of first binding entities to one another and thereby clustering the first cell surface molecules bound to affinity reagents;
c. contacting the cell with a plurality of second binding entities each comprising a second affinity reagent bound to one or more second polymers that are reversibly associative in response to a second stimulus,
i. wherein the cell comprises second cell surface molecules on its surface and contacting the cell with the second binding entities comprises binding a plurality of the second affinity reagents to the cell surface molecules; and
d. applying a second stimulus different than the first stimulus to associate at least some of the plurality of second binding entities to one another and thereby clustering the second cell surface molecules bound to affinity reagents.

107. The method according to claim 106, wherein the first stimulus is a change in temperature across a first temperature threshold and the second stimulus is a change in temperature across a second temperature threshold.

108. The method according to claim 107, wherein the second temperature threshold is higher than the first temperature threshold.

109. A method of co-localizing cells, the method comprising:
a. contacting a first cell with a plurality of binding entities each comprising a first affinity reagent bound to one or more polymers that are reversibly associative in response to a stimulus,
i. wherein the first cell comprises one or more cell surface molecules on its surface and contacting the first cell with the binding entities comprises binding one or more of the first affinity reagents to the one or more cell surface molecules;
b. contacting a second cell with a binding entity comprising a plurality of second affinity reagents bound to a polymer that is reversibly associative in response to a stimulus,
i. wherein the second cell comprises cell surface molecules on its surface and contacting the second cell with the binding entity comprises binding the plurality of the second affinity reagents to the cell surface molecules; and
c. applying a stimulus to associate at least one of the binding entities bound to the first cell and the binding entity bound to the second cell to one another and thereby co-localizing the first and second cells.

110. The method according to claim 109, wherein the first affinity reagent and the second affinity reagent are the same.

111. The method according to claim 109, wherein the first affinity reagent and the second affinity reagent are different.

112. A method of isolating target molecules, the method comprising:
a. contacting a binding entity comprising a plurality of affinity reagents bound to a polymer that is reversibly associative in response to a stimulus with a plurality of target molecules, and thereby binding the plurality of affinity reagents to the target molecules, wherein the binding is in a solution; and
b. applying the stimulus to associate at least some of the plurality of affinity reagents to one another and thereby clustering the target molecules bound to affinity reagents; and
c. separating the binding entity from the solution.

113. The method according to claim 112, wherein separating the binding entity from the solution comprises centrifuging the binding entity and the solution.

114. The method according to claim 112, wherein separating the binding entity from the solution comprises applying an external magnetic field to the solution.

115. The method according to claim 112, wherein separating the binding entity from the solution comprises passing the solution through a chromatography column and retaining the binding entity within the column.

116. The method according to claim 112, wherein the method further comprises isolating the target molecules from the binding entity.

117. The method according to claim 112, wherein the target molecules comprise antibodies.

118. The method according to claim 117, wherein the antibodies are monoclonal.

119. The method according to any one of claims 117-118, wherein the antibodies comprise IgG.

120. The method according to any one of claims 112-119, wherein the affinity reagents comprise protein A.

121. A method of isolating target molecules, the method comprising:
a. contacting a plurality of binding entities each comprising an affinity reagent bound to one or more polymers that are reversibly associative in response to a stimulus with a plurality of target molecules, and thereby binding the affinity reagents to the target molecules, wherein the binding is in a solution;
b. applying the stimulus to associate at least some of the plurality of binding entities to one another and thereby clustering the target molecules bound to affinity reagents; and
c. separating the binding entities from the solution.

122. The method according to claim 121, wherein separating the binding entities from the solution comprises centrifuging the binding entities and the solution.

123. The method according to claim 121, wherein separating the binding entity from the solution comprises applying an external magnetic field to the solution.

124. The method according to claim 121, wherein separating the binding entities from the solution comprises passing the solution through a chromatography column and retaining the binding entities within the column.

125. The method according to claim 121, wherein the method further comprises isolating the target molecules from the binding entities.

126. The method according to claim 121, wherein the target molecules comprise antibodies.

127. The method according to claim 126, wherein the antibodies are monoclonal

128. The method according to any one of claims 126-127, wherein the antibodies comprise IgG.

129. The method according to any one of claims 121-128, wherein the affinity reagent comprises protein A.
